SEQUENCE LISTING (623 sequences)

SEQ ID NO: 1 - protein, 20 aa, Medicago truncatula
CPRVSSHHIECVKGFCTYWK

SEQ ID NO: 2 - protein, 20 aa, Medicago truncatula
IGCDSIYYPISRPCKTDKDC

SEQ ID NO: 3 - protein, 20 aa, Medicago truncatula
CQQKFSTQAEDLLWCIRGYC

SEQ ID NO: 4 - protein, 20 aa, Medicago truncatula
CPQIKSNIFRFKCIEDRCKI

SEQ ID NO: 5 - protein, 20 aa, Medicago truncatula
CAKGDGVCYKSCIEEGFNRG

SEQ ID NO: 6 - protein, 20 aa, Medicago truncatula
LRFRSVFHIFASVLAVAKKH

SEQ ID NO: 7 - protein, 20 aa, Medicago truncatula
CVSKIICVLSQKPLCRNHIC

SEQ ID NO: 8 - protein, 20 aa, Medicago truncatula
YSSCATKEECKCPDNKRPAC

SEQ ID NO: 9 - protein, 20 aa, Medicago truncatula
RGCKRDKDCPQFRGVNIRCR

SEQ ID NO: 10 - protein, 20 aa, Medicago truncatula
VKCVLPRIARCIKYRCQCRN

SEQ ID NO: 11 - protein, 20 aa, Medicago truncatula
LYCNVGSHMECVKHQCKCIK

SEQ ID NO: 12 - protein, 20 aa, Medicago truncatula
CPKFKKYNIRCRKGFCVQVN

SEQ ID NO: 13 - protein, 20 aa, Medicago truncatula
SFHPCKINEHCTTYKCLLTG

SEQ ID NO: 14 - protein, 20 aa, Medicago truncatula
QNLCVGSPLPLQCLKFICRC

SEQ ID NO: 15 - protein, 20 aa, Medicago truncatula
DCKPKRGVNFRCRKGKCYPR

SEQ ID NO: 16 - protein, 20 aa, Medicago truncatula
RKGYTAKRLYEYETQDITQT

SEQ ID NO: 17 - protein, 20 aa, Medicago truncatula
NNFCRYREAVRRLRPPLRKK

SEQ ID NO: 18 - protein, 20 aa, Medicago truncatula
CKTDKECPNTSTHKYKCIND

SEQ ID NO: 19 - protein, 20 aa, Medicago truncatula
CPNGRNYIGRCRKGHCQQRL

SEQ ID NO: 20 - protein, 20 aa, Medicago truncatula
CPDDKKIKGRCRKGFCTNGW

SEQ ID NO: 21 - protein, 20 aa, Medicago truncatula
CKIDKDCPRNPPLNIRCRKS

SEQ ID NO: 22 - protein, 20 aa, Medicago truncatula
DCRRRGSNQYWVYKCINHGC

SEQ ID NO: 23 - protein, 20 aa, Medicago truncatula
CRRKYRGANKHLLWCNDGYC

SEQ ID NO: 24 - protein, 20 aa, Medicago truncatula
DCPKYQRANIRCRKGQCVRI

SEQ ID NO: 25 - protein, 20 aa, Medicago truncatula
KDCPKKMGTVGKCRKGYCAQ

SEQ ID NO: 26 - protein, 20 aa, Medicago truncatula
RPQFRKSNVRCRNGYCVNLG

SEQ ID NO: 27 - protein, 20 aa, Medicago truncatula
GRKCKQNSDCSKEICVFPWK

SEQ ID NO: 28 - protein, 20 aa, Medicago truncatula
KDCPSVKNYIGRCRKGYCQA

SEQ ID NO: 29 - protein, 20 aa, Medicago truncatula
WAYIYVCEKNKCRYHFKSGR

SEQ ID NO: 30 - protein, 20 aa, Medicago truncatula
RRGTNKYFIHKCIDYRCQWI

SEQ ID NO: 31 - protein, 20 aa, Medicago truncatula
KDCPKLGRANIRCREGYCVR

SEQ ID NO: 32 - protein, 20 aa, Medicago truncatula
NYVHKCINNRCEWIKIIRRR

SEQ ID NO: 33 - protein, 20 aa, Medicago truncatula
SFPCKTNSDCPSYLCHYPKN

SEQ ID NO: 34 - protein, 20 aa, Medicago truncatula
CPKVSQYNIRCRKGQCARIR

SEQ ID NO: 35 - protein, 20 aa, Medicago truncatula
RVKRFKCVKGECRWTRMSYA

SEQ ID NO: 36 - protein, 20 aa, Medicago truncatula
DCPQFRRANIRCRKGQCVKL

SEQ ID NO: 37 - protein, 20 aa, Medicago truncatula
DCPKLHRSNVRCRKGQCVQI

SEQ ID NO: 38 - protein, 20 aa, Medicago truncatula
LWDRNYAHRCVNNICEWVKK

SEQ ID NO: 39 - protein, 20 aa, Medicago truncatula
GRTYKCINNKCRYPKLLKPI

SEQ ID NO: 40 - protein, 20 aa, Medicago truncatula
CKQHRGFNFRCRSGNCIPIR

SEQ ID NO: 41 - protein, 20 aa, Medicago truncatula
DCPNMKHYKAKCRKGFCISS

SEQ ID NO: 42 - protein, 20 aa, Medicago truncatula
LWDKNYAHRCVNNICEWVKK

SEQ ID NO: 43 - protein, 20 aa, Medicago truncatula
PPHNIKCRKGHCVPIGKPFK

SEQ ID NO: 44 - protein, 20 aa, Medicago truncatula
CPSWKNYTGRCRKGFCILNR

SEQ ID NO: 45 - protein, 20 aa, Medicago truncatula
CQNHRGFNFRCRKGNCVAKI

SEQ ID NO: 46 - protein, 20 aa, Medicago truncatula
CLALDRKRPIGKKFPCKADK

SEQ ID NO: 47 - protein, 20 aa, Medicago truncatula
CPQKSTHKYYCVDDKCFLYY

SEQ ID NO: 48 - protein, 20 aa, Medicago truncatula
KDCPKLHKVNVRCRKGKCVA

SEQ ID NO: 49 - protein, 20 aa, Medicago truncatula
CPRKNRHVVKCRKGYCVGVQ

SEQ ID NO: 50 - protein, 20 aa, Medicago truncatula
WQKFHTYKCINQKCKWVLRF

SEQ ID NO: 51 - protein, 20 aa, Medicago truncatula
CGSNSDCLWEKCLPPNKHWC

SEQ ID NO: 52 - protein, 20 aa, Medicago truncatula
KDCPKLRGGSFRCRKGKCVL

SEQ ID NO: 53 - protein, 20 aa, Medicago truncatula
SFYKCIDNLCKRFRRQKHLV

SEQ ID NO: 54 - protein, 20 aa, Medicago truncatula
GCIVDPRCPYQQCRRPLYCR

SEQ ID NO: 55 - protein, 20 aa, Medicago truncatula
CKHVRDCPKGIWRSCRYKCI

SEQ ID NO: 56 - protein, 20 aa, Medicago truncatula
CPKVQHGYKLRCRKGQCVHI

SEQ ID NO: 57 - protein, 20 aa, Medicago truncatula
CSSAYTPRCRHRTCVCLNND

SEQ ID NO: 58 - protein, 20 aa, Medicago truncatula
PCATSDDCLKNMCRPPLTPR

SEQ ID NO: 59 - protein, 20 aa, Medicago truncatula
CPPRTRLILYKCRNRKCVSY

SEQ ID NO: 60 - protein, 20 aa, Medicago truncatula
CPRKKKFSVTCRKGFCAEIR

SEQ ID NO: 61 - protein, 20 aa, Medicago truncatula
CPKHRGVNGKCRKGYCVGVG

SEQ ID NO: 62 - protein, 20 aa, Medicago truncatula
FKPKCRFRSCTCSNLKVWKG

SEQ ID NO: 63 - protein, 20 aa, Medicago truncatula
DCPKLRRANVRCRKSYCVPI

SEQ ID NO: 64 - protein, 20 aa, Medicago truncatula
CKETGYTKGGHCRDEGVVCC

SEQ ID NO: 65 - protein, 20 aa, Medicago truncatula
CQKKFPGSNQHLLWCNNGFC

SEQ ID NO: 66 - protein, 20 aa, Medicago truncatula
CSKVEYGYKLRCRKGRCVHI

SEQ ID NO: 67 - protein, 20 aa, Medicago truncatula
FLAESNWSQRNDITKIHCIK

SEQ ID NO: 68 - protein, 20 aa, Medicago truncatula
CVHKRCQLPQIPKCVGKKCR

SEQ ID NO: 69 - protein, 20 aa, Medicago truncatula
CQKKYPGPYEHLLKCVSGYC

SEQ ID NO: 70 - protein, 20 aa, Medicago truncatula
LVTKCFKKHCRCRKPGLQVQ

SEQ ID NO: 71 - protein, 20 aa, Medicago truncatula
CPKVEKPLYMYCGNHWCAYK

SEQ ID NO: 72 - protein, 20 aa, Medicago truncatula
CAPEKYYNIRCRKGFCVQIR

SEQ ID NO: 73 - protein, 20 aa, Medicago truncatula
CRKKFAGANQHLLWCNNGYC

SEQ ID NO: 74 - protein, 20 aa, Medicago truncatula
CYIQYPKTPFGHMECYKGSC

SEQ ID NO: 75 - protein, 20 aa, Medicago truncatula
ANRGLQCLNGECKSSRIIKS

SEQ ID NO: 76 - protein, 20 aa, Medicago truncatula
KCMYKSICKCIREFSKRDYV

SEQ ID NO: 77 - protein, 20 aa, Medicago truncatula
CYKRYPRWSLLPNYCIEGSC

SEQ ID NO: 78 - protein, 20 aa, Medicago truncatula
CPKFYGSNVRCRKGKCVQLG

SEQ ID NO: 79 - protein, 20 aa, Medicago truncatula
DCPAPPRFNIRCRKGYCVRI

SEQ ID NO: 80 - protein, 20 aa, Medicago truncatula
ITPSPKFKWKCINKRCLYIR

SEQ ID NO: 81 - protein, 20 aa, Medicago truncatula
ILCKVHEDCPQKSTHKYYCI

SEQ ID NO: 82 - protein, 20 aa, Medicago truncatula
CPKPPRINIRINIRCRKGFC

SEQ ID NO: 83 - protein, 20 aa, Medicago truncatula
CAYPHVLRCIGKNCAENKNG

SEQ ID NO: 84 - protein, 20 aa, Medicago truncatula
CRPGRIPKCIFGHCNCVKQR

SEQ ID NO: 85 - protein, 20 aa, Medicago truncatula
CPRFKNNNVRCRKGFCVNLC

SEQ ID NO: 86 - protein, 20 aa, Medicago truncatula
IKCKVDEDCPNVFTYSYYKC

SEQ ID NO: 87 - protein, 20 aa, Medicago truncatula
CKSDKDCKDIIIYRYILKCR

SEQ ID NO: 88 - protein, 20 aa, Medicago truncatula
CVTKIKCVLPRKPECRNTQC

SEQ ID NO: 89 - protein, 20 aa, Medicago truncatula
CYKKYPFIPWGKVRCVKGRC

SEQ ID NO: 90 - protein, 20 aa, Medicago truncatula
KINNVRCRKGFCIQIHKFTP

SEQ ID NO: 91 - protein, 20 aa, Medicago truncatula
CDSAYLPLSRSCITDKDCSR

SEQ ID NO: 92 - protein, 20 aa, Medicago truncatula
CKDKFPGNKYPIKCINGIFY

SEQ ID NO: 93 - protein, 20 aa, Medicago truncatula
KIKCVLPRTPQCRNEACGCY

SEQ ID NO: 94 - protein, 20 aa, Medicago truncatula
DCPKFGRVNVRCRKGNCVPI

SEQ ID NO: 95 - protein, 20 aa, Medicago truncatula
RYCVYPTIPLCDVKHCRCRR

SEQ ID NO: 96 - protein, 20 aa, Medicago truncatula
KDCPWAKNYVLKCRKGYCVF

SEQ ID NO: 97 - protein, 20 aa, Medicago truncatula
CKTKVDCPQHKKYIAECIFG

SEQ ID NO: 98 - protein, 20 aa, Medicago truncatula
CKQVKGYIARCRKGYCMQSV

SEQ ID NO: 99 - protein, 20 aa, Medicago truncatula
CTWNLCRQPWVQKCRLHMCS

SEQ ID NO: 100 - protein, 20 aa, Medicago truncatula
CEKIYPGNKKPLICSTGYCY

SEQ ID NO: 101 - protein, 20 aa, Medicago truncatula
IRCRQGLKELASDGLRVTGY

SEQ ID NO: 102 - protein, 20 aa, Medicago truncatula
CTRRKGFSVTCRKGFCVEFK

SEQ ID NO: 103 - protein, 20 aa, Medicago truncatula
CQRYRHKLATRMICNQGFCL

SEQ ID NO: 104 - protein, 20 aa, Medicago truncatula
CPKVEKPITMKCINNYCKYF

SEQ ID NO: 105 - protein, 20 aa, Medicago truncatula
RKCLRSTCVCRKFRFTGFYY

SEQ ID NO: 106 - protein, 20 aa, Medicago truncatula
VHCQKYKCSPGLYPTCINGW

SEQ ID NO: 107 - protein, 20 aa, Medicago truncatula
KQMCHLNQTPKCLKNICKCV

SEQ ID NO: 108 - protein, 20 aa, Medicago truncatula
CKPPFNPRCHNHICICRLWG

SEQ ID NO: 109 - protein, 20 aa, Medicago truncatula
CRAGSHRVQCIKHQCKCVRI

SEQ ID NO: 110 - protein, 20 aa, Medicago truncatula
CPKSVLRVWRCINNYCRPVR

SEQ ID NO: 111 - protein, 20 aa, Medicago truncatula
CPKSEFRKWVCINNICRKMC

SEQ ID NO: 112 - protein, 20 aa, Medicago truncatula
CVTKIKCVLPRKPECRNNAC

SEQ ID NO: 113 - protein, 20 aa, Medicago truncatula
VNNRCYLHGKPSCLNGQCAC

SEQ ID NO: 114 - protein, 20 aa, Medicago truncatula
WTKIYKCIDNKCRYSVVKGL

SEQ ID NO: 115 - protein, 20 aa, Medicago truncatula
DCPKPLRFNIRCRKGFCVRI

SEQ ID NO: 116 - protein, 20 aa, Medicago truncatula
CFIDGNCPRNMCKVRWKLRL

SEQ ID NO: 117 - protein, 20 aa, Medicago truncatula
GLLPRCLNGWCDCSRFQPWP

SEQ ID NO: 118 - protein, 20 aa, Medicago truncatula
LKNEIFHWKKQICNIHFAKL

SEQ ID NO: 119 - protein, 20 aa, Medicago truncatula
RPQCVINTCRCRPLRFSGFY

SEQ ID NO: 120 - protein, 20 aa, Medicago truncatula
IHYYCPPSKVPYCQVDRCGC

SEQ ID NO: 121 - protein, 20 aa, Medicago truncatula
IPRCIKYKCLCGNGVGKRWS

SEQ ID NO: 122 - protein, 20 aa, Medicago truncatula
VYRCVGNYCRAVKIRRWNLG

SEQ ID NO: 123 - protein, 20 aa, Medicago truncatula
CRDYLCARPTVGKCIYDYCH

SEQ ID NO: 124 - protein, 20 aa, Medicago truncatula
KDCRSFLCYSPKFPVCKRGI

SEQ ID NO: 125 - protein, 20 aa, Medicago truncatula
VKIHFYCPPPKVPYCRVDRC

SEQ ID NO: 126 - protein, 20 aa, Medicago truncatula
ISCKDHFECRRKINILRCIY

SEQ ID NO: 127 - protein, 20 aa, Medicago truncatula
CVTRIKCVLPRKPECRNYAC

SEQ ID NO: 128 - protein, 20 aa, Medicago truncatula
ADCPISKLNMYNWRCIKSSC

SEQ ID NO: 129 - protein, 20 aa, Medicago truncatula
CPKYRDLLYVFKCIDKRCEL

SEQ ID NO: 130 - protein, 20 aa, Medicago truncatula
FYVVKCVDHKCELTKKLRRL

SEQ ID NO: 131 - protein, 20 aa, Medicago truncatula
CEFGMIRRCISYKCQCHEAY

SEQ ID NO: 132 - protein, 20 aa, Medicago truncatula
CPNPKYGKCLDNKCICQLIW

SEQ ID NO: 133 - protein, 20 aa, Medicago truncatula
CKHPFKPRCLTHSCVCRLWG

SEQ ID NO: 134 - protein, 20 aa, Medicago truncatula
LANKTFYLKCIDKKCEWTVT

SEQ ID NO: 135 - protein, 20 aa, Medicago truncatula
ADCLGEKCLPPKRYWCRIIT

SEQ ID NO: 136 - protein, 20 aa, Medicago truncatula
CRKHMCTPYGQLVRCINSTC

SEQ ID NO: 137 - protein, 20 aa, Medicago truncatula
VKKPLKMWCIRQTCFYGFGK

SEQ ID NO: 138 - protein, 20 aa, Medicago truncatula
IDKEYTVCSLHSDCKAYVCQ

SEQ ID NO: 139 - protein, 20 aa, Medicago truncatula
KICHPPQIRKCVSKICKCRL

SEQ ID NO: 140 - protein, 20 aa, Medicago truncatula
CVTKVKCGLPRTPKCRNYIC

SEQ ID NO: 141 - protein, 20 aa, Medicago truncatula
FVRCKMNRCIYSRVQPPWAC

SEQ ID NO: 142 - protein, 20 aa, Medicago truncatula
IPYCRFREKNVRFGSPLGLC

SEQ ID NO: 143 - protein, 20 aa, Medicago truncatula
CRTPLKPKCMYRTFCKCKVV

SEQ ID NO: 144 - protein, 20 aa, Medicago truncatula
CRPPLKSKCMYKTNCKCIAV

SEQ ID NO: 145 - protein, 20 aa, Medicago truncatula
CPKLYGANFRCRKGTCVPPI

SEQ ID NO: 146 - protein, 20 aa, Medicago truncatula
RVAWCVNNKCECVLTYGPKY

SEQ ID NO: 147 - protein, 20 aa, Medicago truncatula
CPKSQLEMFAWKCVKNGCHL

SEQ ID NO: 148 - protein, 20 aa, Medicago truncatula
AFYGSMRCVKGFCKHLKDVK

SEQ ID NO: 149 - protein, 20 aa, Medicago truncatula
AQCIYPACFKDHMCRQLKCS

SEQ ID NO: 150 - protein, 20 aa, Medicago truncatula
LKKYIYSPGARRVASSCGVP

SEQ ID NO: 151 - protein, 20 aa, Medicago truncatula
CWPSFKPRCSNGWCVCDKIM

SEQ ID NO: 152 - protein, 20 aa, Medicago truncatula
WFKIYRCEKGICRYHKLWIV

SEQ ID NO: 153 - protein, 20 aa, Medicago truncatula
CPNDCGPHEQAKCILYACYC

SEQ ID NO: 154 - protein, 20 aa, Medicago truncatula
CPPSYTKIYRCIDNKCRLVL

SEQ ID NO: 155 - protein, 20 aa, Medicago truncatula
CITADDCPKVERPLKMKCIG

SEQ ID NO: 156 - protein, 20 aa, Medicago truncatula
CRRNIDCPSYLCVAPKVPRC

SEQ ID NO: 157 - protein, 20 aa, Medicago truncatula
GRMVCGGRSRFGFRSCGGYL

SEQ ID NO: 158 - protein, 20 aa, Medicago truncatula
RCVRRMCKCLPIGWRKYFVP

SEQ ID NO: 159 - protein, 20 aa, Medicago truncatula
ARELPEYLKCQGGMCRLLIK

SEQ ID NO: 160 - protein, 20 aa, Medicago truncatula
GNNLCEGRWIPKCLKPYFLF

SEQ ID NO: 161 - protein, 20 aa, Medicago truncatula
LLKTYVWKCVKNECYFFAKK

SEQ ID NO: 162 - protein, 20 aa, Medicago truncatula
LRANCVDRGVCKCVPVWWRK

SEQ ID NO: 163 - protein, 20 aa, Medicago truncatula
CNVNNICEYNLNVDLVEEIE

SEQ ID NO: 164 - protein, 20 aa, Medicago truncatula
CVARINCVLPRKPQCRNYAC

SEQ ID NO: 165 - protein, 20 aa, Medicago truncatula
IRCVTDADCPNVVKPLKPKC

SEQ ID NO: 166 - protein, 20 aa, Medicago truncatula
KFPGPSKYPIKCMKGICKCV

SEQ ID NO: 167 - protein, 20 aa, Medicago truncatula
CQATTKCVLPRVPRCIKYKC

SEQ ID NO: 168 - protein, 20 aa, Medicago truncatula
LKLKCDRHTILTCFWRHCYC

SEQ ID NO: 169 - protein, 20 aa, Medicago truncatula
VSCKDHYDCRRKVKIVGCIF

SEQ ID NO: 170 - protein, 20 aa, Medicago truncatula
GVFVSCNSHIHCRVNNHKIG

SEQ ID NO: 171 - protein, 20 aa, Medicago truncatula
CKNPDDVPRCIFPVCHCIKS

SEQ ID NO: 172 - protein, 20 aa, Medicago truncatula
IFLCSFIAAKNIDGRNNPTR

SEQ ID NO: 173 - protein, 20 aa, Medicago truncatula
IGCKTSEDCPYLGKCIEDFC

SEQ ID NO: 174 - protein, 20 aa, Medicago truncatula
CIGYWCPLSIQPRSTKPICR

SEQ ID NO: 175 - protein, 20 aa, Medicago truncatula
CVYPYAVQCIHRYCKCLKSR

SEQ ID NO: 176 - protein, 20 aa, Medicago truncatula
FIKCVCGVYGPIRERRLYQS

SEQ ID NO: 177 - protein, 20 aa, Medicago truncatula
VISIHCRTNADCPRNMCKIG

SEQ ID NO: 178 - protein, 20 aa, Medicago truncatula
CTNKIKCVPPRIAQCFRFKC

SEQ ID NO: 179 - protein, 20 aa, Medicago truncatula
CEKLYPGNKKPLICNIGYCL

SEQ ID NO: 180 - protein, 20 aa, Medicago truncatula
LCTSPNEVPECRLLKCQCIK

SEQ ID NO: 181 - protein, 20 aa, Medicago truncatula
GENFCISRFRVKCWRFKCFC

SEQ ID NO: 182 - protein, 20 aa, Medicago truncatula
VGCRLQREKPRCVNLVCRCL

SEQ ID NO: 183 - protein, 20 aa, Medicago truncatula
KTLCHSPGKAKYFCSLLSLK

SEQ ID NO: 184 - protein, 20 aa, Medicago truncatula
VKVSHSHCVIDAHCPRNMCG

SEQ ID NO: 185 - protein, 20 aa, Medicago truncatula
LVQRYRCINGKCNLSFVSYG

SEQ ID NO: 186 - protein, 20 aa, Medicago truncatula
LVQGYRCIDGKCESVFLSYR

SEQ ID NO: 187 - protein, 20 aa, Medicago truncatula
CPKVAKINIRCRRGQCVQVF

SEQ ID NO: 188 - protein, 20 aa, Medicago truncatula
LPYCDRNRFCTSRGYIVCIT

SEQ ID NO: 189 - protein, 20 aa, Medicago truncatula
CYKSKKPLFKIWKCVENVCV

SEQ ID NO: 190 - protein, 20 aa, Medicago truncatula
FCARYPSSKVDYGRCVASIS

SEQ ID NO: 191 - protein, 20 aa, Medicago truncatula
CPYSIQPRSTKPLCRLVGGI

SEQ ID NO: 192 - protein, 20 aa, Medicago truncatula
CRIPLRPKCMYRHICKCKVV

SEQ ID NO: 193 - protein, 20 aa, Medicago truncatula
CKARADCSKLMCELPKISWC

SEQ ID NO: 194 - protein, 20 aa, Medicago truncatula
FIAARVCKSDKDCKDIIIYR

SEQ ID NO: 195 - protein, 20 aa, Medicago truncatula
CQRYRHKLATRMVCNIGFCL

SEQ ID NO: 196 - protein, 20 aa, Medicago truncatula
ATLAFQAAIWKWMTPIKKSQ

SEQ ID NO: 197 - protein, 20 aa, Medicago truncatula
LGMKPKCISVLRMCKCHGWQ

SEQ ID NO: 198 - protein, 20 aa, Medicago truncatula
WICSTGEFPPRALCCRNRCV

SEQ ID NO: 199 - protein, 20 aa, Medicago truncatula
CNHPKIPKCVNNAYCKCVVA

SEQ ID NO: 200 - protein, 20 aa, Medicago truncatula
FCWLDSHMQCIKHQCKCVRI

SEQ ID NO: 201 - protein, 20 aa, Medicago truncatula
IINILCKTDKDCPKVQGANI

SEQ ID NO: 202 - protein, 20 aa, Medicago truncatula
LGEAWFKRTETGEIIWVVRC

SEQ ID NO: 203 - protein, 20 aa, Medicago truncatula
CPKNMCLLTQIPKCFKNVCK

SEQ ID NO: 204 - protein, 20 aa, Medicago truncatula
VNKLRVIKCIDHICQYARNL

SEQ ID NO: 205 - protein, 20 aa, Medicago truncatula
CSKDECPSHLVPKCIGLTCY

SEQ ID NO: 206 - protein, 20 aa, Medicago truncatula
LLKCIHGYCVCFPRNPGDSS

SEQ ID NO: 207 - protein, 20 aa, Medicago truncatula
CRKVRGVNLRCRNGHCVMIL

SEQ ID NO: 208 - protein, 20 aa, Medicago truncatula
QIVKYYCIADQCFYYIKHIK

SEQ ID NO: 209 - protein, 20 aa, Medicago truncatula
AKIDYVLTLKPQCRNYTCVC

SEQ ID NO: 210 - protein, 20 aa, Medicago truncatula
ISCVSDDDCPKVPYPLYIKC

SEQ ID NO: 211 - protein, 20 aa, Medicago truncatula
VCPPNNFVRCIRNLCKCRSL

SEQ ID NO: 212 - protein, 20 aa, Medicago truncatula
GCSFREIPQCINSICKCMKG

SEQ ID NO: 213 - protein, 20 aa, Medicago truncatula
CPTDLTLKCINLTCQCTSEY

SEQ ID NO: 214 - protein, 20 aa, Medicago truncatula
CPKVVKPNYTMCAGGICWQS

SEQ ID NO: 215 - protein, 20 aa, Medicago truncatula
CIRRAGMNIRCRKGYCVNLI

SEQ ID NO: 216 - protein, 20 aa, Medicago truncatula
CRGPMRAKCISKAICKCRLA

SEQ ID NO: 217 - protein, 20 aa, Medicago truncatula
KKPLCYLFASSPFERKIGVC

SEQ ID NO: 218 - protein, 20 aa, Medicago truncatula
CPPIKFAKYLCINYKCRKIC

SEQ ID NO: 219 - protein, 20 aa, Medicago truncatula
ANCPKVISPCHTKCFDGFCG

SEQ ID NO: 220 - protein, 20 aa, Medicago truncatula
LYILSRCVNNICEWVKKPRI

SEQ ID NO: 221 - protein, 20 aa, Medicago truncatula
CRQYLCIPPEFPRCIGGICR

SEQ ID NO: 222 - protein, 20 aa, Medicago truncatula
EIGVRKICIREVCRYFAKIH

SEQ ID NO: 223 - protein, 20 aa, Medicago truncatula
RFMCIPPEKPKCVDLWCKCI

SEQ ID NO: 224 - protein, 20 aa, Medicago truncatula
ISGNLARASRKKPVDVIPCI

SEQ ID NO: 225 - protein, 20 aa, Medicago truncatula
IPCKYNHDCPTILDYISICP

SEQ ID NO: 226 - protein, 20 aa, Medicago truncatula
CPISQLKIYAWKCVKNGCHL

SEQ ID NO: 227 - protein, 20 aa, Medicago truncatula
VKDCPKTVFPIGYKCIKNLC

SEQ ID NO: 228 - protein, 20 aa, Medicago truncatula
CPTGSKPKCVDQVCECILIR

SEQ ID NO: 229 - protein, 20 aa, Medicago truncatula
CSHIRDVICIFKKCKCAGGR

SEQ ID NO: 230 - protein, 20 aa, Medicago truncatula
IGGMFTLKQKKAAKVARSCV

SEQ ID NO: 231 - protein, 20 aa, Medicago truncatula
CPSYLVVKCLRSNCKCVRPG

SEQ ID NO: 232 - protein, 20 aa, Medicago truncatula
CVYPKSMKCIDKKCICVGAR

SEQ ID NO: 233 - protein, 20 aa, Medicago truncatula
CPKIPSLYPTIYKCLDGICR

SEQ ID NO: 234 - protein, 20 aa, Medicago truncatula
FLSIAVSITGNLARASRKKP

SEQ ID NO: 235 - protein, 20 aa, Medicago truncatula
ICLPPKKHWCNILELVRING

SEQ ID NO: 236 - protein, 20 aa, Medicago truncatula
GFCVNSGGATQKCLGCPSLK

SEQ ID NO: 237 - protein, 20 aa, Medicago truncatula
VECPQYSCLRGLKMKCICFK

SEQ ID NO: 238 - protein, 20 aa, Medicago truncatula
CVGKITCVLPQKPECWNYAC

SEQ ID NO: 239 - protein, 20 aa, Medicago truncatula
VSGGFGKCVRDADCVDEVCS

SEQ ID NO: 240 - protein, 20 aa, Medicago truncatula
NCRVKEVGMCYFTKCYCIRL

SEQ ID NO: 241 - protein, 20 aa, Medicago truncatula
CPEKFCSSPDVVRCIYIECY

SEQ ID NO: 242 - protein, 20 aa, Medicago truncatula
IGECYSLYKGKFSLSIISKT

SEQ ID NO: 243 - protein, 20 aa, Medicago truncatula
FFVCPPNNFVRCIRNLCKCR

SEQ ID NO: 244 - protein, 20 aa, Medicago truncatula
LVAKCIKKLCSCRKPGLQIQ

SEQ ID NO: 245 - protein, 20 aa, Medicago truncatula
CIFYGNRIMYRGSRLGICKC

SEQ ID NO: 246 - protein, 20 aa, Medicago truncatula
VSNPPLYFKCIDRGCRIVIK

SEQ ID NO: 247 - protein, 20 aa, Medicago truncatula
CKEAIDCGINFCIRPFKAKC

SEQ ID NO: 248 - protein, 20 aa, Medicago truncatula
NLVCKPGYKLGCSANYQCIC

SEQ ID NO: 249 - protein, 20 aa, Medicago truncatula
FLSLFLMGTSGMKNGCKHTG

SEQ ID NO: 250 - protein, 20 aa, Medicago truncatula
ICSPHEHSKCILYVCYCVDK

SEQ ID NO: 251 - protein, 20 aa, Medicago truncatula
IKKCSSSCRIKCIDFRCLCP

SEQ ID NO: 252 - protein, 20 aa, Medicago truncatula
ADCPKSQVNSFVIKCIKNLC

SEQ ID NO: 253 - protein, 20 aa, Medicago truncatula
CCKALTGADLQCLCSYKNSA

SEQ ID NO: 254 - protein, 20 aa, Medicago truncatula
FCGIWLSKYACGKCLNVTNR

SEQ ID NO: 255 - protein, 20 aa, Medicago truncatula
LRDLVVKCIEGYCKAILYRK

SEQ ID NO: 256 - protein, 20 aa, Medicago truncatula
KILPIIHKCINNFCKLKLYN

SEQ ID NO: 257 - protein, 20 aa, Medicago truncatula
GNTFFIHFKFRSLLCKKHFI

SEQ ID NO: 258 - protein, 20 aa, Medicago truncatula
CGDKIKCVPPRIALCINYKC

SEQ ID NO: 259 - protein, 20 aa, Medicago truncatula
LVSTKILEKHTNKCAATVGL

SEQ ID NO: 260 - protein, 20 aa, Medicago truncatula
IKCVLPRVAKCVRYKCDCVR

SEQ ID NO: 261 - protein, 20 aa, Medicago truncatula
CKTKIKCVLPRIAECVRFKC

SEQ ID NO: 262 - protein, 20 aa, Medicago truncatula
LFYVFICKNRICELINKYPQ

SEQ ID NO: 263 - protein, 20 aa, Medicago truncatula
IIFLSPFLIGRKGGPPGGRT

SEQ ID NO: 264 - protein, 20 aa, Medicago truncatula
CHKVKKPLLLTCIDGICQYT

SEQ ID NO: 265 - protein, 20 aa, Medicago truncatula
IIFISSFIVSKRGGKDKCFR

SEQ ID NO: 266 - protein, 20 aa, Medicago truncatula
WSKFFIYKCVNHVCDSISKV

SEQ ID NO: 267 - protein, 20 aa, Medicago truncatula
CATWINTCIKFKCYCIRPWG

SEQ ID NO: 268 - protein, 20 aa, Medicago truncatula
LLCRNKSELPKCIAGFMCRC

SEQ ID NO: 269 - protein, 20 aa, Medicago truncatula
CAKGFHKLCSFGHCYCITGP

SEQ ID NO: 270 - protein, 20 aa, Medicago truncatula
CNVLYPMYINRRLRCIQGIC

SEQ ID NO: 271 - protein, 20 aa, Medicago truncatula
VKCMLPRIPRCIKYQCLCGY

SEQ ID NO: 272 - protein, 20 aa, Medicago truncatula
CVYPDVFQCINNICKCVSHH

SEQ ID NO: 273 - protein, 20 aa, Medicago truncatula
CDPPEYPRCLGILCKCVYVS

SEQ ID NO: 274 - protein, 20 aa, Medicago truncatula
IFISSFIVSKSLNGGGKDKC

SEQ ID NO: 275 - protein, 20 aa, Medicago truncatula
CPPNYSFLFAIRCIKQKCVT

SEQ ID NO: 276 - protein, 20 aa, Medicago truncatula
KSTCLPPQIPKCLRMICECV

SEQ ID NO: 277 - protein, 20 aa, Medicago truncatula
VRCIRNLCKCRFVYLNTFLK

SEQ ID NO: 278 - protein, 20 aa, Medicago truncatula
CPQLNSEIFAFKCIEKLCKL

SEQ ID NO: 279 - protein, 20 aa, Medicago truncatula
QRRKKSMAKMLKFFFAIILL

SEQ ID NO: 280 - protein, 20 aa, Medicago truncatula
CKKVDQIPKCVGGLCKCFPI

SEQ ID NO: 281 - protein, 20 aa, Medicago truncatula
TSFCIPPQIPKCRTICECIT

SEQ ID NO: 282 - protein, 20 aa, Medicago truncatula
CVRFRCKCVPIGWKNLSHVL

SEQ ID NO: 283 - protein, 20 aa, Medicago truncatula
AKVLKCTPPEVVKCTCLCGK

SEQ ID NO: 284 - protein, 20 aa, Medicago truncatula
CIFTRYFPIYLGGICGCDRK

SEQ ID NO: 285 - protein, 20 aa, Medicago truncatula
IFGQIPKEIGKSLNLKFLSL

SEQ ID NO: 286 - protein, 20 aa, Medicago truncatula
IGRKKKGETPKLVYVIILFL

SEQ ID NO: 287 - protein, 20 aa, Medicago truncatula
IFTKVVGQKFFSFSLDGKCG

SEQ ID NO: 288 - protein, 20 aa, Medicago truncatula
GCCIHGRCWCFNSPIFADKI

SEQ ID NO: 289 - protein, 20 aa, Medicago truncatula
CKTKADCLQHIYYIVECIFG

SEQ ID NO: 290 - protein, 20 aa, Medicago truncatula
CIAQGFVRGGDCIKEDACCC

SEQ ID NO: 291 - protein, 20 aa, Medicago truncatula
CPQLSLRFFAIKCRENVCIY

SEQ ID NO: 292 - protein, 20 aa, Medicago truncatula
CRGICLNSRCVCMMRLGWTY

SEQ ID NO: 293 - protein, 20 aa, Medicago truncatula
FLSIIACNSSFITFRDSRCK

SEQ ID NO: 294 - protein, 20 aa, Medicago truncatula
FLCIIVSNSSFSKTFDRACK

SEQ ID NO: 295 - protein, 20 aa, Medicago truncatula
CKWPFIVQCYKNVIKIGPVG

SEQ ID NO: 296 - protein, 20 aa, Medicago truncatula
CIFDIDCPTKKCAPPLVAKC

SEQ ID NO: 297 - protein, 20 aa, Medicago truncatula
VCLNYKFPTCVGKKCYCLSA

SEQ ID NO: 298 - protein, 20 aa, Medicago truncatula
CYKIYGIPLDGVWRCVKGFC

SEQ ID NO: 299 - protein, 20 aa, Medicago truncatula
CYNQLSCIIEEICLDGSCHC

SEQ ID NO: 300 - protein, 20 aa, Medicago truncatula
CYDQITCIIGDVTCLEGSCD

SEQ ID NO: 301 - protein, 20 aa, Medicago truncatula
LINGGSVPCLTSFGCPRSTC

SEQ ID NO: 302 - protein, 20 aa, Medicago truncatula
CPNVCVDFLISRCINNKCYC

SEQ ID NO: 303 - protein, 20 aa, Medicago truncatula
ANCPCVFPLKPRCNFGYCIC

SEQ ID NO: 304 - protein, 20 aa, Medicago truncatula
VRLPLVPISVGKSIQRFARF

SEQ ID NO: 305 - protein, 20 aa, Medicago truncatula
FISLFFIAKNVEGRVKCIKD

SEQ ID NO: 306 - protein, 20 aa, Medicago truncatula
FLSITVYGYIPGIVNKPCKT

SEQ ID NO: 307 - protein, 20 aa, Medicago truncatula
QQILPRYVLCVNGLCRIYFP

SEQ ID NO: 308 - protein, 20 aa, Medicago truncatula
FMCPSYLAVKCIGRLCRCGR

SEQ ID NO: 309 - protein, 20 aa, Medicago truncatula
KGMCKFPFIVRCLMDQCKCV

SEQ ID NO: 310 - protein, 20 aa, Medicago truncatula
INFCPPGTAPKCFHGLIKCV

SEQ ID NO: 311 - protein, 20 aa, Medicago truncatula
INGVKSLLLIKVRSFIPCQR

SEQ ID NO: 312 - protein, 20 aa, Medicago truncatula
FLSLFLVATNIEGKFQKCCK

SEQ ID NO: 313 - protein, 20 aa, Medicago truncatula
ICVSDKECPTASYPLVCKCI

SEQ ID NO: 314 - protein, 20 aa, Medicago truncatula
CVDSFCVPPNVPKCRVVCKC

SEQ ID NO: 315 - protein, 20 aa, Medicago truncatula
GLAPKCFVSFALARFLSEGR

SEQ ID NO: 316 - protein, 20 aa, Medicago truncatula
GLRRASTITALPINVFAIYK

SEQ ID NO: 317 - protein, 20 aa, Medicago truncatula
LIKIVHLWKKIRVRVDVVKA

SEQ ID NO: 318 - protein, 20 aa, Medicago truncatula
CQSIGCLSHLKPKCTMLGFF

SEQ ID NO: 319 - protein, 20 aa, Medicago truncatula
FGLGRSEAINLNAKNGIIVI

SEQ ID NO: 320 - protein, 20 aa, Medicago truncatula
CTGPQIPKCVSHICFCLSSG

SEQ ID NO: 321 - protein, 20 aa, Medicago truncatula
CPPDMCTLGVIPKCSRFTIC

SEQ ID NO: 322 - protein, 20 aa, Medicago truncatula
CSAENCMVCINFACKCKYSV

SEQ ID NO: 323 - protein, 20 aa, Medicago truncatula
IIYVSLYLVVIEGKDGCKTK

SEQ ID NO: 324 - protein, 20 aa, Medicago truncatula
IAAKNVVKNPGPCFVMGACS

SEQ ID NO: 325 - protein, 20 aa, Medicago truncatula
CPEISIFSPFFYKCINNGCV

SEQ ID NO: 326 - protein, 20 aa, Medicago truncatula
VPLPRFLKCIANLCCLVRKK

SEQ ID NO: 327 - protein, 20 aa, Medicago truncatula
IATGARRKNFFFIILKFSSL

SEQ ID NO: 328 - protein, 20 aa, Medicago truncatula
VRCIKETCKCIKILEPINVV

SEQ ID NO: 329 - protein, 20 aa, Medicago truncatula
IIYVSLYLVVIEGKDGCKTN

SEQ ID NO: 330 - protein, 20 aa, Medicago truncatula
LFLLHIEKSSGVLIDCKTVK

SEQ ID NO: 331 - protein, 20 aa, Medicago truncatula
FLFAMNVAGFGWKCIKRRCV

SEQ ID NO: 332 - protein, 20 aa, Medicago truncatula
LISWTRNNLYIILGLSLFSR

SEQ ID NO: 333 - protein, 20 aa, Medicago truncatula
LFLSLLLVVMGGIRKKECRQ

SEQ ID NO: 334 - protein, 20 aa, Medicago truncatula
FNCPSSLCTFPLKLLKPICT

SEQ ID NO: 335 - protein, 20 aa, Medicago truncatula
IGRKKMGETPKLVYVIILFL

SEQ ID NO: 336 - protein, 20 aa, Medicago truncatula
LPALQRAVMKCIQGFCKIHI

SEQ ID NO: 337 - protein, 20 aa, Medicago truncatula
CPQHLCHELIIPRCKIGVCV

SEQ ID NO: 338 - protein, 20 aa, Medicago truncatula
RSVFCMLATALNSLTWQRLL

SEQ ID NO: 339 - protein, 20 aa, Medicago truncatula
QPWCKLVRLQLLFHGSLIGL

SEQ ID NO: 340 - protein, 20 aa, Medicago truncatula
CRTVADCPKLISSKFVIKCI

SEQ ID NO: 341 - protein, 20 aa, Medicago truncatula
KCIMLKNLSIFSNSGICSCT

SEQ ID NO: 342 - protein, 20 aa, Medicago truncatula
LFCVLPNVPKCIGSKCHCKL

SEQ ID NO: 343 - protein, 20 aa, Medicago truncatula
CPHNLCFPSKAVCISSQCIC

SEQ ID NO: 344 - protein, 20 aa, Medicago truncatula
AFIIFLSIPLPPTRKTIPCK

SEQ ID NO: 345 - protein, 20 aa, Medicago truncatula
FSCLYLFMVTKEVYAKSICK

SEQ ID NO: 346 - protein, 20 aa, Medicago truncatula
VILLLTIFHVSAKKKRYIEC

SEQ ID NO: 347 - protein, 20 aa, Medicago truncatula
CVTPGIPKCTGYVCFCFENL

SEQ ID NO: 348 - protein, 20 aa, Medicago truncatula
GNTTFILFKFLSLFSKQYFI

SEQ ID NO: 349 - protein, 20 aa, Medicago truncatula
FGLVVRCITHHCKCIKILNP

SEQ ID NO: 350 - protein, 20 aa, Medicago truncatula
CIFITRVVPRLRRMGLCSCS

SEQ ID NO: 351 - protein, 20 aa, Medicago truncatula
IIIFLFITEIKGDKFVFDKN

SEQ ID NO: 352 - protein, 20 aa, Medicago truncatula
LGCKVGKVPFLYLGLPVGGN

SEQ ID NO: 353 - protein, 20 aa, Medicago truncatula
CMPGIKPVCSEGWCDCIGFI

SEQ ID NO: 354 - protein, 20 aa, Medicago truncatula
LNKLLIIKCINHVCQYVGNL

SEQ ID NO: 355 - protein, 20 aa, Medicago truncatula
LFLVVTNAGKPFLCTILKKK

SEQ ID NO: 356 - protein, 20 aa, Medicago truncatula
IIFFSLFLVVTNGRSACNRN

SEQ ID NO: 357 - protein, 20 aa, Medicago truncatula
CISLFLVAKNINAIHCNDVN

SEQ ID NO: 358 - protein, 20 aa, Medicago truncatula
VKTKLSMCFLGLRNVAITNV

SEQ ID NO: 359 - protein, 20 aa, Medicago truncatula
CVNLILCDFDEKPKCIINIC

SEQ ID NO: 360 - protein, 20 aa, Medicago truncatula
LMKCVPGKVNVCSLGRCYCV

SEQ ID NO: 361 - protein, 20 aa, Medicago truncatula
ILTPNARKNFLALMHIICGA

SEQ ID NO: 362 - protein, 20 aa, Medicago truncatula
ICSVHAVTKCIGNMCRCLAN

SEQ ID NO: 363 - protein, 20 aa, Medicago truncatula
IIFFSQIIVATNAQKIRRCF

SEQ ID NO: 364 - protein, 20 aa, Medicago truncatula
CVDGFCDVTVKEITKSCFIC

SEQ ID NO: 365 - protein, 20 aa, Medicago truncatula
AIIYVSMYLVVIEGKDGCKT

SEQ ID NO: 366 - protein, 20 aa, Medicago truncatula
LVANGLKIFCIDVADCPKDL

SEQ ID NO: 367 - protein, 20 aa, Medicago truncatula
LLYKCIYNKCIVFTRIPFPF

SEQ ID NO: 368 - protein, 20 aa, Medicago truncatula
IIIYLFLLRVVAKDLHRRQL

SEQ ID NO: 369 - protein, 18 aa, Medicago truncatula
MGETILVGVVKAIANRVH

SEQ ID NO: 370 - protein, 20 aa, Medicago truncatula
CLPSLVSKCINFICECTHSM

SEQ ID NO: 371 - protein, 20 aa, Medicago truncatula
IFVSLFLVATKGGSKPFLTR

SEQ ID NO: 372 - protein, 20 aa, Medicago truncatula
FLSLFLVIINCRFELKTSSK

SEQ ID NO: 373 - protein, 20 aa, Medicago truncatula
LTIFISLLLIETIRRLQCKH

SEQ ID NO: 374 - protein, 20 aa, Medicago truncatula
IFLYLFHVATTNRFLYRIGC

SEQ ID NO: 375 - protein, 20 aa, Medicago truncatula
VINVGGKCISHMHCIYLSCG

SEQ ID NO: 376 - protein, 20 aa, Medicago truncatula
ILCLFPIIKRCIHNHCKCVP

SEQ ID NO: 377 - protein, 20 aa, Medicago truncatula
FCSTFEILSIERKVGVCECI

SEQ ID NO: 378 - protein, 20 aa, Medicago truncatula
FLSIIISNSSFGMIFDRACK

SEQ ID NO: 379 - protein, 20 aa, Medicago truncatula
ILYLFLLHVIAEDFPFHKCE

SEQ ID NO: 380 - protein, 20 aa, Medicago truncatula
IVLFLSLFLATKNIDGRRVS

SEQ ID NO: 381 - protein, 20 aa, Medicago truncatula
FLVIRVSDSIPYVNIGPCVK

SEQ ID NO: 382 - protein, 20 aa, Medicago truncatula
LVVVCHDHICKCLRLIKIRS

SEQ ID NO: 383 - protein, 20 aa, Medicago truncatula
LYIACVAHECQCVHLQSALT

SEQ ID NO: 384 - protein, 20 aa, Medicago truncatula
LSVLLVVIEGYPFQECKVDA

SEQ ID NO: 385 - protein, 20 aa, Medicago truncatula
IIVCDSSIIFLRCITDKDCP

SEQ ID NO: 386 - protein, 20 aa, Medicago truncatula
IYLFLLHVVAKDLPFNICEK

SEQ ID NO: 387 - protein, 20 aa, Medicago truncatula
CKPFCNQAFLSCCAFGQCIC

SEQ ID NO: 388 - protein, 20 aa, Medicago truncatula
GLVVRCITRQCKCITILNPI

SEQ ID NO: 389 - protein, 20 aa, Medicago truncatula
GFVVAVKCVRRLCIYNVHLH

SEQ ID NO: 390 - protein, 20 aa, Medicago truncatula
ILLSQFLVKKAEITNIPCVS

SEQ ID NO: 391 - protein, 20 aa, Medicago truncatula
LQPKCIVLEILPHSLSGGIC

SEQ ID NO: 392 - protein, 20 aa, Medicago truncatula
LLFLFRLLIVKEVSGKSKLY

SEQ ID NO: 393 - protein, 20 aa, Medicago truncatula
LIIFLSIYVGVNDCKRIPCK

SEQ ID NO: 394 - protein, 20 aa, Medicago truncatula
VYVTIIFLYMFHISTNIEGK

SEQ ID NO: 395 - protein, 20 aa, Medicago truncatula
LKFVVTKKKTLKFVYAMILF

SEQ ID NO: 396 - protein, 20 aa, Medicago truncatula
VFLFLSIFLSAGNSKSYGPC

SEQ ID NO: 397 - protein, 20 aa, Medicago truncatula
IICIYLFIVITTRKTDIRCR

SEQ ID NO: 398 - protein, 20 aa, Medicago truncatula
ICATHGISKCVATMCFCNLN

SEQ ID NO: 399 - protein, 20 aa, Medicago truncatula
VLFLFISTPFIIKKPGSPNL

SEQ ID NO: 400 - protein, 20 aa, Medicago truncatula
AKCFVSFALARFLSKGKCLC

SEQ ID NO: 401 - protein, 20 aa, Medicago truncatula
LIIFLSLFLVESKQTNIPCK

SEQ ID NO: 402 - protein, 20 aa, Medicago truncatula
ISFISLFFIAKNDAVYIKCK

SEQ ID NO: 403 - protein, 20 aa, Medicago truncatula
IQMGKNMAQRFMFIYALIIF

SEQ ID NO: 404 - protein, 20 aa, Medicago truncatula
KLHFPLLFLLLTLFTTKPLQ

SEQ ID NO: 405 - protein, 20 aa, Medicago truncatula
CLFPYLVVTTKTAIACVTNK

SEQ ID NO: 406 - protein, 20 aa, Medicago truncatula
AFIIFLSIHLPTVRSDIPCK

SEQ ID NO: 407 - protein, 20 aa, Medicago truncatula
FLVIIVSHSVTSPWVLKQHC

SEQ ID NO: 408 - protein, 20 aa, Medicago truncatula
IYKCIPPAKPKCVLFGCMCI

SEQ ID NO: 409 - protein, 20 aa, Medicago truncatula
LIFLTLFVVALSNDTEYTDC

SEQ ID NO: 410 - protein, 20 aa, Medicago truncatula
ILFISLLLVVTKGYREPFSS

SEQ ID NO: 411 - protein, 20 aa, Medicago truncatula
VILVSLFFVIANSRGIRPGR

SEQ ID NO: 412 - protein, 20 aa, Medicago truncatula
GRNMTIKTLKFVYVIILFFS

SEQ ID NO: 413 - protein, 20 aa, Medicago truncatula
FSLFLVAKGDDVKIKCVSAI

SEQ ID NO: 414 - protein, 20 aa, Medicago truncatula
IFLSLFLVTTKAAERIYRCL

SEQ ID NO: 415 - protein, 20 aa, Medicago truncatula
VCSVPGCSNICTLPDVPTCI

SEQ ID NO: 416 - protein, 20 aa, Medicago truncatula
IIFLSISFSITNSFKMFCRY

SEQ ID NO: 417 - protein, 20 aa, Medicago truncatula
IIILSLFQLSINAREKVNCL

SEQ ID NO: 418 - protein, 20 aa, Medicago truncatula
IICVFLLNIAAQEIENGIHP

SEQ ID NO: 419 - protein, 20 aa, Medicago truncatula
FIFLIILSAKVRGAHIKCET

SEQ ID NO: 420 - protein, 20 aa, Medicago truncatula
FILISLFLVVTNANANNCTD

SEQ ID NO: 421 - protein, 20 aa, Medicago truncatula
ILFLFLFFVTTEACGGKTHY

SEQ ID NO: 422 - protein, 20 aa, Medicago truncatula
CPYLISRTLVIMCINKQCVA

SEQ ID NO: 423 - protein, 20 aa, Medicago truncatula
FVYAFFIFLSIAHRPPANTI

SEQ ID NO: 424 - protein, 20 aa, Medicago truncatula
IGLIPCVSDADCPEELALVM

SEQ ID NO: 425 - protein, 20 aa, Medicago truncatula
LRRKNTVQILMFVSALLIYI

SEQ ID NO: 426 - protein, 20 aa, Medicago truncatula
IFMLPFVMRCINFRCQIVNS

SEQ ID NO: 427 - protein, 20 aa, Medicago truncatula
FVYAFIIFLSIHFPPRIKCN

SEQ ID NO: 428 - protein, 20 aa, Medicago truncatula
IIFLSLSLVAIEAGRGYRCT

SEQ ID NO: 429 - protein, 20 aa, Medicago truncatula
VLSDGVCMSLSGTFNGLCIP

SEQ ID NO: 430 - protein, 20 aa, Medicago truncatula
GGNMTNIIKFVKVMIYFLSI

SEQ ID NO: 431 - protein, 20 aa, Medicago truncatula
ILFVSLCLVVVDGESKLEQT

SEQ ID NO: 432 - protein, 20 aa, Medicago truncatula
FIFLIILPAKIRGETLSLTH

SEQ ID NO: 433 - protein, 20 aa, Medicago truncatula
IFLSLFLVESEKLDIRCATV

SEQ ID NO: 434 - protein, 20 aa, Medicago truncatula
LLNGKIIYLLCLKKKKFLII

SEQ ID NO: 435 - protein, 20 aa, Medicago truncatula
CPEAVFFVTFRCIKNICVRI

SEQ ID NO: 436 - protein, 20 aa, Medicago truncatula
FIFLIILPAKIRGEVFQRVT

SEQ ID NO: 437 - protein, 20 aa, Medicago truncatula
LRRKNTVQILMFVSALIIYI

SEQ ID NO: 438 - protein, 20 aa, Medicago truncatula
FFYALIIFLSPFLVDRRSFP

SEQ ID NO: 439 - protein, 20 aa, Medicago truncatula
IFLTLFVVALSDDSKPFSSL

SEQ ID NO: 440 - protein, 20 aa, Medicago truncatula
IQFISLFLITIEVGRLRYGC

SEQ ID NO: 441 - protein, 20 aa, Medicago truncatula
LIILFSPFLAARLVFVNREK

SEQ ID NO: 442 - protein, 20 aa, Medicago truncatula
GFQIGCVRKICTCLRILAPI

SEQ ID NO: 443 - protein, 20 aa, Medicago truncatula
IFLFLVANNVEGYILCKTVN

SEQ ID NO: 444 - protein, 20 aa, Medicago truncatula
CPKAVSFLVFKCIDNICVRV

SEQ ID NO: 445 - protein, 20 aa, Medicago truncatula
ILISLFLVVTNANAHNCTDI

SEQ ID NO: 446 - protein, 20 aa, Medicago truncatula
IIFLSLFLLSTNIDAAECYQ

SEQ ID NO: 447 - protein, 20 aa, Medicago truncatula
ILFSLFVFFTSGAIPCGTRD

SEQ ID NO: 448 - protein, 20 aa, Medicago truncatula
ILLIFVIFASDMCKKSAARG

SEQ ID NO: 449 - protein, 20 aa, Medicago truncatula
IKFILFLLDIPNIIPCKTRV

SEQ ID NO: 450 - protein, 20 aa, Medicago truncatula
KILKFVYEMILFLSLFHLAR

SEQ ID NO: 451 - protein, 20 aa, Medicago truncatula
IIIFISLFLGANVEGRIKCK

SEQ ID NO: 452 - protein, 20 aa, Medicago truncatula
LSLFLVAKGDDVKIKCVVAA

SEQ ID NO: 453 - protein, 20 aa, Medicago truncatula
FVHVLIIFLSLFHVVKNDDG

SEQ ID NO: 454 - protein, 20 aa, Medicago truncatula
CDNVICVAGGIPKCITPFCF

SEQ ID NO: 455 - protein, 20 aa, Medicago truncatula
FVSLYLVVVDGVSKLAQSCS

SEQ ID NO: 456 - protein, 20 aa, Medicago truncatula
GESMAKIVKFVYFVIIFASP

SEQ ID NO: 457 - protein, 20 aa, Medicago truncatula
ILFLSLFLVAKNVTAQIRCN

SEQ ID NO: 458 - protein, 20 aa, Medicago truncatula
VSKFFIFSKFSCLLIHNLLY

SEQ ID NO: 459 - protein, 20 aa, Medicago truncatula
ILFISLLLVVTGAVRKPECR

SEQ ID NO: 460 - protein, 20 aa, Medicago truncatula
ASTFRLTFFFLIVNAVNIRC

SEQ ID NO: 461 - protein, 20 aa, Medicago truncatula
IIFASLYYVVALVQNECVTD

SEQ ID NO: 462 - protein, 20 aa, Medicago truncatula
LFITLFLVAKNVDALKKCIT

SEQ ID NO: 463 - protein, 20 aa, Medicago truncatula
ILVSLIIVATSHSFLPCQTK

SEQ ID NO: 464 - protein, 20 aa, Medicago truncatula
FVYAFIIFLSIPLPPARSDF

SEQ ID NO: 465 - protein, 20 aa, Medicago truncatula
LCLLLVTMNVNAVIKCFQDS

SEQ ID NO: 466 - protein, 20 aa, Medicago truncatula
CLPPLQVICGGDFLCFCIYQ

SEQ ID NO: 467 - protein, 20 aa, Medicago truncatula
GIRKNMAEILKFVYIMIIFL

SEQ ID NO: 468 - protein, 20 aa, Medicago truncatula
CMIIFLSLFLIATKVGGEHN

SEQ ID NO: 469 - protein, 20 aa, Medicago truncatula
IILLSQFIVEKAEITNIPCV

SEQ ID NO: 470 - protein, 20 aa, Medicago truncatula
FIVLVTLFLGPKNVYAFQPC

SEQ ID NO: 471 - protein, 20 aa, Medicago truncatula
IIILNHMNVKLMKIVHKILL

SEQ ID NO: 472 - protein, 20 aa, Medicago truncatula
ILLHSLFLIVIDTTNGLKCG

SEQ ID NO: 473 - protein, 20 aa, Medicago truncatula
FSLFLVATNAGGCNPCLVTC

SEQ ID NO: 474 - protein, 20 aa, Medicago truncatula
AIFFVSLIFGVVSGKKKCAS

SEQ ID NO: 475 - protein, 20 aa, Medicago truncatula
INFLSLFLVETAITNIRCVS

SEQ ID NO: 476 - protein, 20 aa, Medicago truncatula
LIILFSPFLAARLVFVNPEK

SEQ ID NO: 477 - protein, 20 aa, Medicago truncatula
FGLLCIAFVLASGPTPSSLH

SEQ ID NO: 478 - protein, 20 aa, Medicago truncatula
FIYSLIIFLSPFLGEAVFKR

SEQ ID NO: 479 - protein, 20 aa, Medicago truncatula
ILVLFLFFVATKVDGAVHKE

SEQ ID NO: 480 - protein, 20 aa, Medicago truncatula
LIFSIFLCTSISIFACKTDK

SEQ ID NO: 481 - protein, 20 aa, Medicago truncatula
IIFLSLFMVEANIPGARCAT

SEQ ID NO: 482 - protein, 20 aa, Medicago truncatula
VSVFLIVVYGEKECISDAVC

SEQ ID NO: 483 - protein, 20 aa, Medicago truncatula
VVTLIICLKLTYISPNLKYL

SEQ ID NO: 484 - protein, 20 aa, Medicago truncatula
IIFVYSLNSVTIHMFIKLST

SEQ ID NO: 485 - protein, 20 aa, Medicago truncatula
ILLVTLFLVPKNVDAFVKCE

SEQ ID NO: 486 - protein, 20 aa, Medicago truncatula
ILISLFLFSTNVDGKPIFIS

SEQ ID NO: 487 - protein, 20 aa, Medicago truncatula
KFVKVMIYFLSIFLISTYFK

SEQ ID NO: 488 - protein, 20 aa, Medicago truncatula
IIFLSLSFVVTSYRTRIPCV

SEQ ID NO: 489 - protein, 20 aa, Medicago truncatula
ILFLSLFLITTNVGGSYYGC

SEQ ID NO: 490 - protein, 20 aa, Medicago truncatula
ISLFLVAGGEEIIIIKCQTA

SEQ ID NO: 491 - protein, 20 aa, Medicago truncatula
ILLSIFLVEKAEITNIPCVS

SEQ ID NO: 492 - protein, 20 aa, Medicago truncatula
FLYFSNFLLYFVVHTQSFVL

SEQ ID NO: 493 - protein, 20 aa, Medicago truncatula
FFISLFLALVHVDGARFGIK

SEQ ID NO: 494 - protein, 20 aa, Medicago truncatula
ALISFLSLILVLSSNEIEHC

SEQ ID NO: 495 - protein, 20 aa, Medicago truncatula
VSVLIIFLSLFLADTKQTNI

SEQ ID NO: 496 - protein, 20 aa, Medicago truncatula
LKALLIFFSLFLVEINGEVK

SEQ ID NO: 497 - protein, 20 aa, Medicago truncatula
IIFLFLILDVATYAEKIRTC

SEQ ID NO: 498 - protein, 20 aa, Medicago truncatula
ILFSPFLAALVIIDHHKPCV

SEQ ID NO: 499 - protein, 20 aa, Medicago truncatula
LIIFFFLFLVETKRTNIPCF

SEQ ID NO: 500 - protein, 20 aa, Medicago truncatula
IVLLSQFLVVINGSIPCETT

SEQ ID NO: 501 - protein, 20 aa, Medicago truncatula
IVILLLLLVATEAGTGNIRQ

SEQ ID NO: 502 - protein, 20 aa, Medicago truncatula
GKYMAQILKFVYVIIIFLSS

SEQ ID NO: 503 - protein, 20 aa, Medicago truncatula
ILFLFLFAINVTAFRDPCNF

SEQ ID NO: 504 - protein, 20 aa, Medicago truncatula
FFTIFSIFVFYTTFYHLTLT

SEQ ID NO: 505 - protein, 20 aa, Medicago truncatula
FIFLFLVAKNVKGYVVCRTV

SEQ ID NO: 506 - protein, 20 aa, Medicago truncatula
LLFSIFLCISISIFSCKTDK

SEQ ID NO: 507 - protein, 20 aa, Medicago truncatula
FLSLFLIAIDIKVEAFLRCD

SEQ ID NO: 508 - protein, 20 aa, Medicago truncatula
ILFLSLLLVVMGGIRRFECR

SEQ ID NO: 509 - protein, 20 aa, Medicago truncatula
IFLSLFIVAMNANAFSICQN

SEQ ID NO: 510 - protein, 20 aa, Medicago truncatula
FPNLFYRIFYHLLVTFFIFF

SEQ ID NO: 511 - protein, 20 aa, Medicago truncatula
IIFLSLFLVATNVNAINKCS

SEQ ID NO: 512 - protein, 20 aa, Medicago truncatula
IFISLILSVANAGKSLFSDI

SEQ ID NO: 513 - protein, 20 aa, Medicago truncatula
LIIFFSLFPVITNGDRIPCV

SEQ ID NO: 514 - protein, 20 aa, Medicago truncatula
ILFASLFLVAMEIGGQSFLR

SEQ ID NO: 515 - protein, 20 aa, Medicago truncatula
ALIIFVSPFLLATFRTRLPC

SEQ ID NO: 516 - protein, 20 aa, Medicago truncatula
IIIFSVFFVTTKSDSILCTT

SEQ ID NO: 517 - protein, 20 aa, Medicago truncatula
FVYTLIIFLFPSHVITNKIA

SEQ ID NO: 518 - protein, 20 aa, Medicago truncatula
VIVLFLSLFLAAKNIDGRVS

SEQ ID NO: 519 - protein, 20 aa, Medicago truncatula
FKFVYTIIIYLFLLRVVAKD

SEQ ID NO: 520 - protein, 20 aa, Medicago truncatula
LLSIFLIKIVSGSNTLLAFR

SEQ ID NO: 521 - protein, 20 aa, Medicago truncatula
FIYSLIIFLSLFFGEAALER

SEQ ID NO: 522 - protein, 20 aa, Medicago truncatula
FLYALITFLFLFLVETSTTN

SEQ ID NO: 523 - protein, 20 aa, Medicago truncatula
QIRKIMSGVLKFVYAIILFL

SEQ ID NO: 524 - protein, 20 aa, Medicago truncatula
VLFLSLFIFSIAAQNLMKCN

SEQ ID NO: 525 - protein, 20 aa, Medicago truncatula
LCVFGSKAECVVNICICVPP

SEQ ID NO: 526 - protein, 20 aa, Medicago truncatula
IIFISLILDVTNAGPIFCYN

SEQ ID NO: 527 - protein, 20 aa, Medicago truncatula
IILFSPLIAQRIVGMSLFTP

SEQ ID NO: 528 - protein, 20 aa, Medicago truncatula
IIFISLFLVVTTHIPCVHHD

SEQ ID NO: 529 - protein, 20 aa, Medicago truncatula
FISLFLVSKNVAIDIFVCQT

SEQ ID NO: 530 - protein, 20 aa, Medicago truncatula
IIFLSQFLVVTSTTTFPCVS

SEQ ID NO: 531 - protein, 20 aa, Medicago truncatula
IYALIIFSSLFVRDGIPCLS

SEQ ID NO: 532 - protein, 20 aa, Medicago truncatula
FISLILIVTSNVHSLLPCGT

SEQ ID NO: 533 - protein, 20 aa, Medicago truncatula
VFISLFFVVRDVKAGLAHFC

SEQ ID NO: 534 - protein, 20 aa, Medicago truncatula
FIFLFLVANNVEGYIVCITD

SEQ ID NO: 535 - protein, 20 aa, Medicago truncatula
FIFLFLVSTNVHAGIRCVFP

SEQ ID NO: 536 - protein, 20 aa, Medicago truncatula
VILFLSLFLVVTNVESADCD

SEQ ID NO: 537 - protein, 20 aa, Medicago truncatula
IIFFSLFFVLTNGELEIRCV

SEQ ID NO: 538 - protein, 20 aa, Medicago truncatula
VLFLSLFLIATDVKAFLKCD

SEQ ID NO: 539 - protein, 20 aa, Medicago truncatula
ILFSPSLVVPLKVIIPSSTC

SEQ ID NO: 540 - protein, 20 aa, Medicago truncatula
KTIKFVYTMILFLSLFIVAK

SEQ ID NO: 541 - protein, 20 aa, Medicago truncatula
KIFKFIYGLVIFLYLFLIQK

SEQ ID NO: 542 - protein, 20 aa, Medicago truncatula
FDPILVTSFYYLLFFLTAVT

SEQ ID NO: 543 - protein, 20 aa, Medicago truncatula
ILFIFLFLVAYKIEALTKCE

SEQ ID NO: 544 - protein, 20 aa, Medicago truncatula
ILINISLFFVEATELNIPCV

SEQ ID NO: 545 - protein, 20 aa, Medicago truncatula
ILFLFLFLVAAEDIGGNCEC

SEQ ID NO: 546 - protein, 20 aa, Medicago truncatula
FYGSIIFLSLFLLAAFFEKG

SEQ ID NO: 547 - protein, 20 aa, Medicago truncatula
ILFVSLLLIVVASERECVTD

SEQ ID NO: 548 - protein, 20 aa, Medicago truncatula
ILFISLILVVTGIKADTSCH

SEQ ID NO: 549 - protein, 20 aa, Medicago truncatula
IFYAFIIFLCVFFVPTKSSK

SEQ ID NO: 550 - protein, 20 aa, Medicago truncatula
IFLSLFLIEASIKTKIACVT

SEQ ID NO: 551 - protein, 20 aa, Medicago truncatula
LFLFLIAMNVNALYVCRKVA

SEQ ID NO: 552 - protein, 20 aa, Medicago truncatula
CAIILFLSLFLVTYFERFGP

SEQ ID NO: 553 - protein, 20 aa, Medicago truncatula
CLTILFLSLFLVAAEEDIGG

SEQ ID NO: 554 - protein, 20 aa, Medicago truncatula
IVFYTLFLVATEIVSGIPCN

SEQ ID NO: 555 - protein, 20 aa, Medicago truncatula
VIILILSLFLVAKGGGKKIY

SEQ ID NO: 556 - protein, 20 aa, Medicago truncatula
FLKFVYVFIIFLAIRLPPAK

SEQ ID NO: 557 - protein, 20 aa, Medicago truncatula
IILISLFLFSTNVDGKPIFI

SEQ ID NO: 558 - protein, 20 aa, Medicago truncatula
ILFVSLFLIVVDVCGKCNSD

SEQ ID NO: 559 - protein, 20 aa, Medicago truncatula
VSLCLVVVDGISIYVRCAST

SEQ ID NO: 560 - protein, 20 aa, Medicago truncatula
VFLCLSIFLVVESLNFGPCN

SEQ ID NO: 561 - protein, 20 aa, Medicago truncatula
FSLVLLILFITQLHNKVAAA

SEQ ID NO: 562 - protein, 20 aa, Medicago truncatula
ALTIFLSQLLVAASSLCISD

SEQ ID NO: 563 - protein, 20 aa, Medicago truncatula
IICLFPYLVVTFKTAITCDC

SEQ ID NO: 564 - protein, 20 aa, Medicago truncatula
IISISLFLVVTNGVKIPCVK

SEQ ID NO: 565 - protein, 20 aa, Medicago truncatula
AFIIFLSLFFVLTKSSIPCK

SEQ ID NO: 566 - protein, 20 aa, Medicago truncatula
ILFLSLFLFIKNVDGAFVKC

SEQ ID NO: 567 - protein, 20 aa, Medicago truncatula
FLSIFIIVTNGGLIPCVSDA

SEQ ID NO: 568 - protein, 20 aa, Medicago truncatula
ALTIFLSLFIVGAVRIPRPL

SEQ ID NO: 569 - protein, 20 aa, Medicago truncatula
FYALLIFVSLFLVTTNGSLP

SEQ ID NO: 570 - protein, 20 aa, Medicago truncatula
AKTLKVMYTMVLFFSLFLVA

SEQ ID NO: 571 - protein, 20 aa, Medicago truncatula
GEIIKFVYSRIIFLSLFLLA

SEQ ID NO: 572 - protein, 20 aa, Medicago truncatula
IFLSLLLILTDGGLINGGSV

SEQ ID NO: 573 - protein, 16 aa, Medicago truncatula
MTSIVIKYFWALLLYL

SEQ ID NO: 574 - protein, 20 aa, Medicago truncatula
ILSLFIAVTNALIFCFEDIN

SEQ ID NO: 575 - protein, 20 aa, Medicago truncatula
FFSLFPVITNGGIYISFLFF

SEQ ID NO: 576 - protein, 20 aa, Medicago truncatula
IIVFYTLFLVGTEIVSGHAC

SEQ ID NO: 577 - protein, 20 aa, Medicago truncatula
VHALILFLSLFLVAKVRTPC

SEQ ID NO: 578 - protein, 20 aa, Medicago truncatula
FVYTLILYLFLLYVVPFHRC

SEQ ID NO: 579 - protein, 20 aa, Medicago truncatula
VIIIFLSLFLVATNIKGKPF

SEQ ID NO: 580 - protein, 20 aa, Medicago truncatula
IHALVIFLSLIGLVISGNHT

SEQ ID NO: 581 - protein, 20 aa, Medicago truncatula
ISVFALILILSPFLVVTDRD

SEQ ID NO: 582 - protein, 20 aa, Medicago truncatula
VVLFFCILSFSAKTLARNIV

SEQ ID NO: 583 - protein, 20 aa, Medicago truncatula
AKTLKFLCGLVLFVYLFFIK

SEQ ID NO: 584 - protein, 20 aa, Medicago truncatula
IVFFFIFLSVTNSSAFSGCM

SEQ ID NO: 585 - protein, 20 aa, Medicago truncatula
ILVSLILVVTSHSFLPCVTK

SEQ ID NO: 586 - protein, 20 aa, Medicago truncatula
IIFFSLILAVTNAGLFRCKV

SEQ ID NO: 587 - protein, 20 aa, Medicago truncatula
FIIFLSLILAVISQHPFTPC

SEQ ID NO: 588 - protein, 20 aa, Medicago truncatula
ALIIFLSFILAVISEDIENC

SEQ ID NO: 589 - protein, 20 aa, Medicago truncatula
ILILCVSLLLIGEASGKECV

SEQ ID NO: 590 - protein, 20 aa, Medicago truncatula
FLSLFLVITNSVRIPCVTVA

SEQ ID NO: 591 - protein, 20 aa, Medicago truncatula
VQTPFFIYAFIIFLSLFPYV

SEQ ID NO: 592 - protein, 20 aa, Medicago truncatula
IMILCVSLLLIAEASGKECV

SEQ ID NO: 593 - protein, 20 aa, Medicago truncatula
FILTMILLLSLFLVAESGGK

SEQ ID NO: 594 - protein, 20 aa, Medicago truncatula
LILFISLILVVTGINAEADT

SEQ ID NO: 595 - protein, 20 aa, Medicago truncatula
LFVSIFIIVVNVGGKCVSDA

SEQ ID NO: 596 - protein, 20 aa, Medicago truncatula
IILLSLFLAAIDADVVNCTS

SEQ ID NO: 597 - protein, 20 aa, Medicago truncatula
ILFLSLFLVQFLTCKGLTVP

SEQ ID NO: 598 - protein, 20 aa, Medicago truncatula
ALIIVLSLFLVETNTATCIT

SEQ ID NO: 599 - protein, 20 aa, Medicago truncatula
IICLFLLQVAAQEVLVIHEC

SEQ ID NO: 600 - protein, 20 aa, Medicago truncatula
FIVVSLFLVVTCETRIPCVS

SEQ ID NO: 601 - protein, 20 aa, Medicago truncatula
ALIIFISLVITGRSTINVMC

SEQ ID NO: 602 - protein, 20 aa, Medicago truncatula
VKTLKFVNVIIFFLSLFLSA

SEQ ID NO: 603 - protein, 19 aa, Medicago truncatula
MDALITLIVQLIYVSLHLF

SEQ ID NO: 604 - protein, 20 aa, Medicago truncatula
IFLSLFLVATKAEFTLFILS

SEQ ID NO: 605 - protein, 20 aa, Medicago truncatula
CVRILFISLFLIATKFGVAS

SEQ ID NO: 606 - protein, 20 aa, Medicago truncatula
FLSIFIITLQVNVVVCEIDA

SEQ ID NO: 607 - protein, 20 aa, Medicago truncatula
LIIFLSIYLVVTDGIILCKD

SEQ ID NO: 608 - protein, 20 aa, Medicago truncatula
NNIVKFGYVMIIFLSLFFVV

SEQ ID NO: 609 - protein, 20 aa, Medicago truncatula
IIILSLALVVTCNGIPICQT

SEQ ID NO: 610 - protein, 20 aa, Medicago truncatula
LVKFVYVMITLLSIVVVAKN

SEQ ID NO: 611 - protein, 20 aa, Medicago truncatula
KIIKFVYVLAIFFSLFLVAK

SEQ ID NO: 612 - protein, 20 aa, Medicago truncatula
GQILIFVFALINFLSPILVE

SEQ ID NO: 613 - protein, 20 aa, Medicago truncatula
GQIQKFISSLIIIISLVLVV

SEQ ID NO: 614 - protein, 20 aa, Medicago truncatula
VKVFKFTYLMIIFFSLFLVA

SEQ ID NO: 615 - protein, 20 aa, Medicago truncatula
LILFYSIFLGIIVCNSSLIS

SEQ ID NO: 616 - protein, 20 aa, Medicago truncatula
KILKCVYAMILFLPLFVVAM

SEQ ID NO: 617 - protein, 20 aa, Medicago truncatula
IICLFLFLLHVAAQKDLIIL

SEQ ID NO: 618 - protein, 20 aa, Medicago truncatula
FLLFIYSLIIFLSLFFGEAA

SEQ ID NO: 619 - protein, 20 aa, Medicago truncatula
FKILLFTSSIIVFLSLFFVT

SEQ ID NO: 620 - protein, 20 aa, Medicago truncatula
FFAIILLLSLFLVATEVGGA

SEQ ID NO: 621 - protein, 20 aa, Medicago truncatula
AKVLKLVNVMIIFLALVLVA

SEQ ID NO: 622 - protein, 17 aa, Medicago truncatula
KWCFRVCYRGICYRRCR

SEQ ID NO: 623 - protein, 18 aa, Medicago truncatula
RRWCFRVCYRGFCYRKCR

(19) United States
(12) Patent Application Publication
(10) Pub. No.: US 2024/0349723 A1
(43) Pub. Date: Oct. 24, 2024

(54) PLANT-BASED PEPTIDES FOR TREATMENT AND PREVENTION OF CITRUS GREENING DISEASE

(71) Applicant: The United States of America, as represented by the Secretary of Agriculture, Washington, DC (US)

(72) Inventors: MICHELLE L. HECK, ITHACA, NY (US); LAURA FLEITES, PORT ST LUCIA, FL (US); STEVEN HIGGINS, COLUMBUS, OH (US); STACY L. DEBLASIO, ITHACA, NY (US); JOHN S. RAMSEY, ITHACA, NY (US); ROBERT G. SHATTERS, FORT PIERCE, FL (US)

(21) Appl. No.: 18/621,166

(22) Filed: Mar. 29, 2024

Related U.S. Application Data
(60) Provisional application No. 63/496,373, filed on Apr. 14, 2023.

(51) Int. Cl. A01N 37/46; A01N 65/00; A01P 1/00; A01P 7/04; C12N 15/82

(52) U.S. Cl. CPC A01N 37/46; A01N 65/00; A01P 1/00; A01P 7/04; C12N 15/8281

(57) ABSTRACT

Compositions and methods using plant-derived peptides, that are biopesticides are described. The compositions and methods can be used to reduce the damage of citrus plants and plant parts as well as losses in harvested fruits caused by citrus greening disease by treatment with a peptide or mixture of peptides.

### CROSS-REFERENCE TO RELATED APPLICATIONS

This application claims the benefit of U.S. Provisional Application No. 63/496,373, titled “PLANT-BASED PEPTIDES FOR TREATMENT AND PREVENTION OF CITRUS GREENING DISEASE” filed Apr. 14, 2023, which is incorporated herein by reference in its entirety.

### FIELD OF THE INVENTION

The invention described herein relates to novel pest and pathogen control compositions and methods comprising peptide compositions for the control of pests and pathogens causing citrus greening disease.

### SEQUENCE LISTING

The instant application contains a Sequence Listing XML required by 37 C.F.R. § 1.831 (a) which has been submitted in XML file format via the USPTO patent electronic filing system, and is hereby incorporated by reference in its entirety. The XML file was created on Mar. 28, 2024, is named Sequence_Listing_002123, and has 535 kilobytes.

### BACKGROUND OF THE INVENTION

Huanglongbing (HLB), also known as citrus greening disease, is currently the most devastating disease affecting citrus production worldwide. Infection is mainly confined to vascular tissue, specifically the phloem.

HLB represents a devastating, recalcitrant pathosystem that has resulted in significant economic losses for the United States citrus industry. It was first reported in China in 1919 (but was likely circulating there in the 1800s) and the African strain was first reported in 1937 in South Africa, where it is now widespread. It has continued to spread across the globe and pose a serious threat to citrus production. It has decimated production in Florida and Puerto Rico.

The putative causal bacteria of HLB are phloem-limited, gram-negative Alpha-proteobacteria in the genus Liberibacter and include ‘Candidatus Liberibacter asiaticus’ (CLas), found in Asia, North and South America, Oceania and the Arabian Peninsula (Bové, 2006; Haapalainen, 2014); ‘Ca. Liberibacter americanus’ (CLam), found in South America (Texeira et al., 2005; Teixeira et al., 2008); and ‘Ca. Liberibacter africanus (CLaf) found in Africa and the Arabian Peninsula (Garnier and Bové, 1996; Pietersen et al., 2010). These three bacteria can be transmitted from plant to plant by grafting or dodder (plant parasite), but their natural spread is by insect vectors (da Graca et al., 2016). Two psyllid vectors of HLB-associated Liberibacter spp. have been identified: the Asian citrus psyllid, Diaphorina citri Kuwayama (Hemiptera: Liviidae), (Capoor et al., 1967; Bové, 2006), and the African citrus psyllid, Trioza erytreae del Guercio (Hemiptera: Triozidae). D. citri is the most widely spread vector of CLas, which is also the most wide-spread bacterium related to HLB worldwide.

There has been a long felt, critical and unmet need for new compositions and methods of treating diseases such as HLB, citrus stubborn disease, etc. for the maintenance of citrus production in the US and worldwide.

The compositions and methods of treatment described herein address some of these important problems.

### SUMMARY OF THE INVENTION

Described herein are compositions and methods using plant-derived active peptides, that are biopesticides.

In an aspect, compositions and methods herein reduce damage of citrus plants and plant parts as well as losses in harvested fruits caused by citrus greening disease by treatment of a plant in need thereof with a composition comprising a peptide or mixture of peptides.

In one aspect, a peptide composition is provided with pesticidal activity with a sequence

(SEQ ID NO: 8)
YSSCATKEECKCPDNKRPAC.

In one aspect, a peptide composition is provided with pesticidal activity with a sequence

(SEQ ID NO: 9)
RGCKRDKDCPQFRGVNIRCR.

In one aspect, a peptide composition is provided with pesticidal activity with a sequence

(SEQ ID NO: 10)
VKCVLPRIARCIKYRCQCRN.

In one aspect, a peptide composition is provided with pesticidal activity with a sequence

(SEQ ID NO: 11)
LYCNVGSHMECVKHQCKCIK.

In one aspect, a peptide composition is provided with a sequence QNLCVGSPLPLQCLKFICRC (SEQ ID NO: 14) with pesticidal activity.

The present disclosure provides biopesticide peptide compositions having amino acid sequences disclosed as SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, and SEQ ID NO: 14 or mixtures thereof.

Methods are described herein for reducing damage of citrus plants and plant parts as well as losses in harvested fruits by treatment of a plant in need thereof with effective amounts of a composition comprising a peptide with a peptide sequence that is disclosed as SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14 or mixtures thereof.

In one aspect, the compositions and methods of treatment described herein relate to plant-derived active peptide sequences (SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14 or mixtures thereof) which are biopesticides having killing-activity against CLas and D. citri and are also effective at inhibiting the growth, movement, morphology acquisition and/or transmission of CLas in citrus trees.

In another aspect, the compositions and methods of treatment described herein relate to plant-derived active peptide 20-mer sequences bearing 20 amino acid residues exemplified by SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14 or mixtures thereof where the peptide can be a 20-mer which is part of a 21-mer, 22-mer or part of a larger polypeptide which are biopesticides having killing-activity against CLas and optionally D. citri and effective at inhibiting the growth, movement, morphology acquisition and/or transmission of CLas in citrus trees.

### BRIEF DESCRIPTION OF THE DRAWINGS

FIG. 1 Average growth rate inhibition of Liberibacter crescens strain BT-1 in BM7 medium by 128 plant-derived active peptides.

FIG. 2 Growth rate inhibition as a function of the physiochemical properties of the plant-derived active peptides.

FIG. 3 Effect of the top 14 plant-derived active peptides (SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14) on CLas in citrus leaves, nymph acquisition and on the growth of L. crescens.

FIG. 4 Example of citrus excised leaf assay set up.

FIG. 5 Excised leaf delivery of plant-derived active peptides to target CLas in planta shows impacts on CLas titer for SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14).

FIG. 6 Excised leaf acquisition assay demonstrates impacts on CLas titer in leaves (A) and psyllids (B) following treatment with five plant-derived active peptides, SEQ ID NO: 14, SEQ ID NO 11, SEQ ID NO 10, SEQ ID NO: 9 and SEQ ID NO: 8.

FIG. 7 Leaf delivery of five plant-derived active peptides reduces the number of infective Diaphorina citri adults that develop on treated leaves (SEQ ID NO: 14, SEQ ID NO: 11, SEQ ID NO: 10, SEQ ID NO: 9 and SEQ ID NO: 8). No highly infected adults develop on leaves treated with plant-derived active peptide sequences SEQ ID NO: 10 (803364) and SEQ ID NO: 9 (803543).

FIG. 8 Welch's ANOVA (p=7.7312e-017) followed by Dunnett's T3 multiple comparison's test on CLas cell equivalents* in Diaphorina citri individuals following acquisition from infected leaves for plant-derived active peptides derived peptides (SEQ ID NO: 10, SEQ ID NO: 9 and SEQ ID NO: 8).

FIG. 9 Plant-derived active peptides have pesticidal activity against D. citri, an insect vector of citrus greening disease

### DETAILED DESCRIPTION

Described herein are compositions and methods using plant-derived active peptide sequences, that are biopesticides. Compositions and methods herein can reduce damage of citrus plants and plant parts as well as losses in harvested fruits by treatment of citrus greening disease, or Huanglongbing (HLB) with a composition comprising a peptide or mixture of peptides.

Also provided herein are modified peptide compositions for treating citrus greening disease, or Huanglongbing (HLB) comprising a polypeptide having at least 85% identity to SEQ ID NO: 8, 85% identity to SEQ ID NO: 9, 85% identity to SEQ ID NO: 10, 85% identity to SEQ ID NO: 11, or 85% identity to SEQ ID NO: 14 wherein a peptide with 95%, 90% or 85% identity comprises an amino acid substitution in any one, two or three of residues 1-20 of the peptide respectively.

The present disclosure additionally provides a method of controlling citrus greening disease, or Huanglongbing (HLB), where the method has the step of exposing a plant to a peptide having an amino acid sequence at least 85% identical to SEQ ID NO: 8. Peptide SEQ ID NO: 8 with 95%, 90% or 85% identity can have an amino acid substitution in any one, two or three of residues 1-20.

The present disclosure additionally provides a method of controlling citrus greening disease, or Huanglongbing (HLB), where the method has at least the step of exposing a plant to a peptide having an amino acid sequence at least 85% identical to SEQ ID NO: 9. Peptide SEQ ID NO: 9 de with 95%, 90% or 85% identity has an amino acid substitution in any one, two or three of residues 1-20 respectively.

The present disclosure additionally provides a method of controlling citrus greening disease, or Huanglongbing (HLB), where the method has at least the step of exposing a plant to a peptide having an amino acid sequence at least 85% identical to SEQ ID NO: 10. Peptide SEQ ID NO: 10 with 95%, 90% or 85% identity has an amino acid substitution in any one, two or three of residues 1-20 respectively.

The present disclosure additionally provides a method of controlling citrus greening disease, or Huanglongbing (HLB), where the method has at least the steps of exposing a plant to a peptide having an amino acid sequence at least 85% identical to SEQ ID NO: 11. Peptide SEQ ID NO: 11 with 95%, 90% or 85% identity has an amino acid substitution in any one, two or three of residues 1-20 respectively.

The present disclosure additionally provides a method of controlling citrus greening disease, or Huanglongbing (HLB), where the method has at least the steps of exposing a plant to a peptide having an amino acid sequence at least 85% identical to SEQ ID NO: 14. Peptide SEQ ID NO: 14 with 95%, 90% or 85% identity has an amino acid substitution in any one, two or three of residues 1-20 respectively.

In another aspect the peptide composition is modified by acetylation of an amino residue. In another embodiment, an N-terminal residue amino acid is acetylated providing peptide compositions and methods for controlling citrus greening disease.

In one aspect, compositions and methods can use one or more acetylated amino residue peptides of the various sequences described herein with at least one of the acetylations on an N-terminal amino acid.

In other aspects, the plant-derived active peptide compositions are modified with an amidated CONH2 C-terminus residue.

In other aspects, the plant-derived active peptide compositions are modified with cyclization by a disulfide bridge of two cystine residues of the peptide.

In other aspects, the plant-derived active peptide compositions are modified with N- and/or C-terminal modification or substitution, D-amino acid or unnatural amino acid substitution, cyclization, backbone modification, nanoparticle formulations and increased molecular mass. In other aspects a plurality of the above stabilizing modifications are applied to a plant-derived active peptide for the treatment of citrus greening disease.

The present disclosure additionally provides a method of controlling citrus greening disease, or Huanglongbing (HLB), where the method has at least the step of treating a plant with a peptide having an amino acid sequence at least 85% identical to SEQ ID NO: 8 and has said peptide with 95%, 90% or 85% identity with an amino acid substitution in any one, two or three of residues 1-20.

The present disclosure additionally provides a method of controlling citrus greening disease, or Huanglongbing (HLB), where the method has at least the step of treating a plant with a peptide having an amino acid sequence at least 85% identical to SEQ ID NO: 9 and has said peptide with 95%, 90% or 85% identity with an amino acid substitution in any one, two or three of residues 1-20.

The present disclosure additionally provides a method of controlling citrus greening disease, or Huanglongbing (HLB), where the method has at least the step of treating a plant with a peptide having an amino acid sequence at least 85% identical to SEQ ID NO: 10 and has said peptide with 95%, 90% or 85% identity with an amino acid substitution in any one, two or three of residues 1-20.

The present disclosure additionally provides a method of controlling citrus greening disease, or Huanglongbing (HLB), where the method has at least the step of treating a plant with a peptide having an amino acid sequence at least 85% identical to SEQ ID NO: 11 and has said peptide with 95%, 90% or 85% identity with an amino acid substitution in any one, two or three of residues 1-20.

The present disclosure additionally provides a method of controlling citrus greening disease, or Huanglongbing (HLB), where the method has at least the step of treating a plant with a peptide having an amino acid sequence at least 85% identical to SEQ ID NO: 14 and has said peptide with 95%, 90% or 85% identity with an amino acid substitution in any one, two or three of residues 1-20.

The present disclosure provides biopesticide peptide compositions for treating citrus greening disease, or Huanglongbing (HLB) having amino acid sequences selected from SEQ ID NO: 1 to SEQ ID NO: 623 or mixtures thereof.

In one aspect, an antibacterial pesticide composition with a peptide having an amino acid sequence of SEQ ID NO: 8 is provided. In one aspect, an antibacterial pesticide composition with a peptide having an amino acid sequence of SEQ ID NO: 8 having an N-terminal acetylated peptide residue is provided. In one aspect, an antibacterial pesticide composition with a peptide having an amino acid sequence of SEQ ID NO: 8 with an amidated CONH2 C-terminus residue is provided. In one aspect, an antibacterial pesticide composition with a peptide having an amino acid sequence of SEQ ID NO: 8 with a disulfide bridge of two cystine residues in the peptide sequence is provided.

In one aspect, an antibacterial pesticide composition with a peptide having an amino acid sequence of SEQ ID NO: 9 is provided. In one aspect, an antibacterial pesticide composition with a peptide having an amino acid sequence of SEQ ID NO: 9 having an N-terminal acetylated peptide residue is provided. In one aspect, an antibacterial pesticide composition with a peptide having an amino acid sequence of SEQ ID NO: 9 with an amidated CONH2 C-terminus residue is provided. In one aspect, an antibacterial pesticide composition with a peptide having an amino acid sequence of SEQ ID NO: 9 with a disulfide bridge of two cystine residues in the peptide sequence is provided.

In one aspect, an antibacterial pesticide composition with a peptide having an amino acid sequence of SEQ ID NO: 10 is provided. In one aspect, an antibacterial pesticide composition with a peptide having an amino acid sequence of SEQ ID NO: 10 having an N-terminal acetylated peptide residue is provided. In one aspect, an antibacterial pesticide composition with a peptide having an amino acid sequence of SEQ ID NO: 10 with an amidated CONH2 C-terminus residue is provided. In one aspect, an antibacterial pesticide composition with a peptide having an amino acid sequence of SEQ ID NO: 10 with a disulfide bridge of two cystine residues in the peptide sequence is provided.

In one aspect, an antibacterial pesticide composition with a peptide having an amino acid sequence of SEQ ID NO: 11 is provided. In one aspect, an antibacterial pesticide composition with a peptide having an amino acid sequence of SEQ ID NO: 11 with an amidated CONH2 C-terminus residue is provided. In one aspect, an antibacterial pesticide composition with a peptide having an amino acid sequence of SEQ ID NO: 11 with a disulfide bridge of two cystine residues in the peptide sequence is provided.

In one aspect, an antibacterial pesticide composition with a peptide having an amino acid sequence of SEQ ID NO: 14 is provided. In one aspect, an antibacterial pesticide composition with a peptide having an amino acid sequence of SEQ ID NO: 14 having an N-terminal acetylated peptide residue is provided. In one aspect, an antibacterial pesticide composition with a peptide having an amino acid sequence of SEQ ID NO: 14 with an amidated CONH2 C-terminus residue is provided. In one aspect, an antibacterial pesticide composition with a peptide having an amino acid sequence of SEQ ID NO: 14 with a disulfide bridge of two cystine residues in the peptide sequence is provided.

In one aspect, an antibacterial pesticide composition with a peptide having an amino acid sequence of SEQ ID NO: 11 to treat citrus greening disease is provided. In one aspect, an antibacterial pesticide composition with a peptide having an amino acid sequence of SEQ ID NO: 11 having an N-terminal acetylated peptide residue is provided.

In one aspect, an antibacterial pesticide composition with a peptide having an amino acid sequence of SEQ ID NO: 14 to treat citrus greening disease is provided. In one aspect, an antibacterial pesticide composition with a peptide having an amino acid sequence of SEQ ID NO: 14 having an N-terminal acetylated peptide residue is provided.

In one aspect, an antibacterial pesticide composition with a peptide having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12. SEQ ID NO: 13, SEQ ID NO: 14 or mixtures thereof to treat citrus greening disease is provided.

In one aspect, an insecticidal pesticide composition with a peptide having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12. SEQ ID NO: 13, SEQ ID NO: 14 or mixtures thereof to treat citrus greening disease is provided.

In one aspect, an antibacterial pesticide composition with a peptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12. SEQ ID NO: 13, SEQ ID NO: 14 or mixtures thereof with an acetylated residue is provided.

In one aspect, an antibacterial pesticide composition with a peptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12. SEQ ID NO: 13, SEQ ID NO: 14 or mixtures thereof with an N-terminal acetylated peptide is provided.

In one aspect, an antibacterial pesticide composition with a peptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12. SEQ ID NO: 13, SEQ ID NO: 14 or mixtures thereof with an amidated CONH2 C-terminus residue is provided.

In one aspect, an antibacterial pesticide composition with a peptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12. SEQ ID NO: 13, SEQ ID NO: 14 or mixtures thereof with a cyclization by a disulfide bridge of two cystine residues in the peptide sequence is provided.

In one aspect, an antibacterial pesticide composition with a peptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12. SEQ ID NO: 13, SEQ ID NO: 14 or mixtures thereof with one or more of N- and/or C-terminal modification or substitution, D-amino acid or unnatural amino acid substitution, cyclization, backbone modification or nanoparticle formulation is provided.

In one aspect, an antibacterial pesticide composition with a peptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12. SEQ ID NO: 13, SEQ ID NO: 14 or mixtures thereof where the peptides are recombinantly expressed in a host cell, with a plant cell being one example host cell is provided.

In one aspect, a peptide composition selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12. SEQ ID NO: 13, SEQ ID NO: 14 or mixtures thereof for treating citrus greening disease is provided.

In one aspect, a peptide composition selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12. SEQ ID NO: 13, SEQ ID NO: 14 or mixtures thereof with a peptide having an amino acid sequence at least 85% identical to said sequence is provided.

In one aspect, a peptide composition selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12. SEQ ID NO: 13, SEQ ID NO: 14 or mixtures thereof with a peptide having an amino acid residue substitution in at least one of residues 1-20 is provided.

In one aspect, a composition of any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12. SEQ ID NO: 13, SEQ ID NO: 14 or mixtures thereof with a peptide having an amino acid residue substitution in at least two of residues 1-20 is provided.

In one aspect, a composition of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12. SEQ ID NO: 13, SEQ ID NO: 14 or mixtures thereof with a peptide having an amino acid residue substitution in at least three of residues 1-20 is provided.

In one aspect, a method for treating citrus greening disease by treatment of a plant in need thereof with a composition comprising an effective amount of a peptide with SEQ ID NO: 8 is provided.

In one aspect, a method for treating citrus greening disease by treatment of a plant in need thereof with a composition comprising an effective amount of a peptide with SEQ ID NO: 9 is provided.

In one aspect, a method for treating citrus greening disease by treatment of a plant in need thereof with a composition comprising an effective amount of a peptide with SEQ ID NO: 10 is provided.

In one aspect, a method for treating citrus greening disease by treatment of a plant in need thereof with a composition comprising an effective amount of a peptide with SEQ ID NO: 11 is provided.

In one aspect, a method for treating citrus greening disease by treatment of a plant in need thereof with a composition comprising an effective amount of a peptide with SEQ ID NO: 14 is provided.

In one aspect, a method for killing D. citri, vector of citrus greening disease by treatment of a plant in need thereof with a composition comprising an effective amount of a peptide with SEQ ID NO: 11 is provided.

In one aspect, a method for killing a D. citri, vector of citrus greening disease by treatment of a plant in need thereof with a composition comprising an effective amount of a peptide with SEQ ID NO: 14 is provided.

In one aspect, a method of prophylactic treatment of citrus greening disease by treatment of a plant in need thereof with a composition comprising an effective amount of a peptide with SEQ ID NO: 14. SEQ ID NO: 11, SEQ ID NO: 10, SEQ ID NO: 9 or SEQ ID NO: 8.

In one aspect, a method for killing a D. citri, vector of citrus greening disease by treatment of a plant in need thereof with a composition comprising an effective amount of a peptide with SEQ ID NO: 14. SEQ ID NO: 11, SEQ ID NO: 10, SEQ ID NO: 9 or SEQ ID NO: 8 with the peptide modified by having acetylated residue is provided.

In one aspect, a method for treating citrus greening disease by treatment of a plant in need thereof with a composition comprising an effective amount of a peptide with SEQ ID NO: 14. SEQ ID NO: 11, SEQ ID NO: 10, SEQ ID NO: 9 or SEQ ID NO: 8 with the peptide modified by having acetylated residue is provided.

In one aspect, a method for treating citrus greening disease by treatment of a plant in need thereof with a composition comprising an effective amount of a peptide with SEQ ID NO: 14. SEQ ID NO: 11, SEQ ID NO: 10, SEQ ID NO: 9 or SEQ ID NO: 8 with the peptide modified by having an N-terminal acetylated peptide is provided.

In one aspect, a method for treating citrus greening disease by treatment of a plant in need thereof with a composition comprising an effective amount of a peptide with SEQ ID NO: 14. SEQ ID NO: 11, SEQ ID NO: 10, SEQ ID NO: 9 or SEQ ID NO: 8 with the peptide modified by having an amidated CONH2 C-terminus residue is provided.

In one aspect, a method for treating citrus greening disease by treatment of a plant in need thereof with a composition comprising an effective amount of a peptide with SEQ ID NO: 14. SEQ ID NO: 11, SEQ ID NO: 10, SEQ ID NO: 9 or SEQ ID NO: 8 with the peptide modified by having a cyclization by a disulfide bridge of two cystine residues is provided.

In one aspect, a method for treating citrus greening disease by treatment of a plant in need thereof with a composition comprising an effective amount of a peptide with SEQ ID NO: 14. SEQ ID NO: 11, SEQ ID NO: 10, SEQ ID NO: 9 or SEQ ID NO: 8 with the peptide having a N- and/or C-terminal modification or substitution, D-amino acid or unnatural amino acid substitution, cyclization, backbone modification or nanoparticle formulation is provided.

In one aspect, a method for treating citrus greening disease by treatment of a plant in need thereof with a composition comprising an effective amount of a peptide with SEQ ID NO: 14. SEQ ID NO: 11, SEQ ID NO: 10, SEQ ID NO: 9 or SEQ ID NO: 8 with the peptide recombinantly expressed in a host cell, with a plant cell being one example host cell.

In one aspect, a method for treating citrus greening disease by treatment of a plant in need thereof with a composition comprising an effective amount of a peptide with SEQ ID NO: 14. SEQ ID NO: 11, SEQ ID NO: 10, SEQ ID NO: 9 or SEQ ID NO: 8 applied to the citrus foliar surfaces of the plant is provided.

The term “effective amount” or “effective dosage” is defined as an amount sufficient to achieve or at least partially achieve the desired effect. The term “therapeutically effective amount” is defined as an amount sufficient to cure or at least partially arrest the disease and its complications in a plant already effected from the disease. Amounts or doses effective for this use will depend on the condition to be treated (the indication), the delivered peptide construct, the therapeutic context and objectives, the severity of the disease, prior treatments, etc. The proper dose can be adjusted according to the judgment of those skilled in the art such that it can be administered to the plant once or over a series of administrations, and to obtain the optimal therapeutic effect.

A typical treatment dosage may range from about 1 gram/acre to 1000 grams/acre or in a range of 0.1 μg/kg plant weight to up to about 30 mg/kg plant weight or more, depending on the factors mentioned above. In specific embodiments, the dosage may range from 1.0 μg/kg plant weight up to about 20 mg/kg plant weight, optionally from 10 μg/kg up to about 10 mg/kg or from 100 μg/kg up to about 500 mg/kg plant weight.

In various aspects, the compositions described herein can be administered by injection using various methods and apparatus known in the art. For example, U.S. Pat. No. 5,597,840 describes injection methods and is incorporated herein by reference.

In other aspects, a peptide composition comprising a compound with an amino acid sequence of SEQ ID NO. 1 to SEQ ID NO. 623 as described herein may be applied to a plant by any means described herein guarding it from or preventing the spread or occurrence of citrus greening disease or infection employing a prophylactic method of treatment. In other aspects, a composition comprising a compound as described herein may be supplied to a plant exogenously. The composition may be applied to the plant and/or the surrounding soil through sprays, drips, and/or other forms of liquid application.

The compounds described herein may penetrate the citrus plant through the roots via the soil (systemic action); by drenching the locus of the plant with a liquid composition; or by applying the compounds in solid form to the soil, e.g. in granular form (soil application).

As used herein, the term “locus” broadly encompasses the fields on which the treated plants are growing, or where the seeds of cultivated plants are sown, or the place where the seed will be placed into the soil.

For example, in some aspects, a composition is applied to a citrus plant, including plant leaves, shoots, roots or seeds. In one aspect, a composition comprising a compound as described herein is applied to a foliar surface of a plant. In some aspects, foliar applications may require 10 to 500 grams per hectare of a composition as described herein.

As used herein, the term “foliar surface” broadly refers to any green portion of a plant having surface that may permit absorption of a composition, including petioles, stipules, stems, bracts, flowerbuds, and leaves. Absorption commonly occurs at the site of application on a foliar surface, but in some cases, the applied composition may run down to other areas and be absorbed there.

The compositions described herein can be applied to the citrus foliar surfaces of the plant using any conventional system for applying liquids to a foliar surface. For example, in some embodiments, application by spraying will be found most convenient. Any conventional atomization method can be used to generate spray droplets, including hydraulic nozzles and rotating disk atomizers. In some embodiments, alternative application techniques, including application by brush or by rope-wick, may be utilized.

In some embodiments, a composition comprising a compound as described herein is directly applied to the soil surrounding the root zone of a plant. Soil applications may require 0.1 to 5 kg per hectare of a composition as described herein on a broadcast basis (rate per treated area if broadcast or banded).

For example, in some embodiments, a composition may be applied directly to the base of the plants or to the soil immediately adjacent to the plants.

In some embodiments, a sufficient quantity of the composition is applied such that it drains through the soil to the root area of the plants.

Generally, application of the compositions described herein may be performed using any method or apparatus known in the art, including but not limited to injection under ambient or elevated pressures, hand sprayer, mechanical sprinkler, or irrigation, including drip irrigation.

For exogenous delivery to citrus, provided herein are compositions as described above additionally comprising at least one auxiliary selected from the group consisting of extenders, solvents, spontaneity promoters, carriers, emulsifiers, dispersants, frost protectants, thickeners and adjuvants. Those compositions are referred to as formulations.

Accordingly, in one aspect, provided herein are compositions with such formulations, and application forms prepared from them, are provided as pesticidal agents, such as drench, drip, direct injection and spray liquors, comprising the compositions described herein. The application forms may comprise further pesticidal agents, and/or activity-enhancing adjuvants such as penetrants, examples being vegetable oils such as, for example, rapeseed oil, sunflower oil, mineral oils such as, for example, liquid paraffins, alkyl esters of vegetable fatty acids, such as rapeseed oil or soybean oil methyl esters, or alkanol alkoxylates, and/or spreaders such as, for example, alkylsiloxanes and/or salts, examples being organic or inorganic ammonium or phosphonium salts, examples being ammonium sulphate or diammonium hydrogen phosphate, and/or retention promoters such as dioctyl sulphosuccinate or hydroxypropylguar polymers and/or humectants such as glycerol and/or fertilizers such as ammonium, potassium or phosphorous fertilizers, for example. In an advantageous embodiment, said peptide compositions have stimulation properties of plant natural defenses and/or fungicide properties. Said peptides can thus be applied by different ways on the surface of the plants, in particular by spraying on the leaves and/or the stem.

In various aspects, examples of typical formulations include water-soluble liquids (SL), emulsifiable concentrates (EC), emulsions in water (EW), suspension concentrates (SC, SE, FS, OD), water-dispersible granules (WG), granules (GR) and capsule concentrates (CS); these and other possible types of formulation are described, for example, by Crop Life International and in Pesticide Specifications, Manual on development and use of FAO and WHO specifications for pesticides, FAO Plant Production and Protection Papers-173, prepared by the FAO/WHO Joint Meeting on Pesticide Specifications, 2004, ISBN: 9251048576. The formulations may comprise active agrochemical compounds other than one or more active compounds of the compositions described herein.

The formulations or application forms in question can comprise auxiliaries, such as extenders, solvents, spontaneity promoters, carriers, emulsifiers, dispersants, frost protectants, biocides, thickeners and/or other auxiliaries, such as adjuvants, for example. An adjuvant in this context is a component which enhances the biological effect of the formulation, without the component itself having a biological effect. Examples of adjuvants are agents which promote the retention, spreading, attachment to the leaf surface, or penetration.

These formulations are produced in a known manner, for example by mixing the active compounds with auxiliaries such as, for example, extenders, solvents and/or solid carriers and/or further auxiliaries, such as, for example, surfactants. The formulations are prepared either in suitable plants or else before or during the application.

Suitable for use as auxiliaries are substances which are suitable for imparting to the formulation of the active compound or the application forms prepared from these formulations particular properties such as certain physical, technical and/or biological properties.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly) ethers, the unsubstituted and substituted amines, amides, lactams (such as N-alkylpyrrolidones) and lactones, the sulphones and sulphoxides (such as dimethyl sulphoxide).

If the extender used is water, it is also possible to employ, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethylformamide and dimethyl sulphoxide, and also water. In one aspect, preferred auxiliary solvents are selected from the group consisting of acetone and N,N′-dimethylformamide.

In principle it is possible to use various suitable solvents for compositions described herein, in different aspects. Suitable solvents are, for example, potassium phosphate buffer, aromatic hydrocarbons, such as xylene, toluene or alkylnaphthalenes, for example, chlorinated aromatic or aliphatic hydrocarbons, such as chlorobenzene, chloroethylene or methylene chloride, for example, aliphatic hydrocarbons, such as cyclohexane, for example, paraffins, petroleum fractions, mineral and vegetable oils, alcohols, such as methanol, ethanol, isopropanol, butanol or glycol, for example, and also their ethers and esters, ketones such as acetone, methyl ethyl ketone, methyl isobutyl ketone or cyclohexanone, for example, strongly polar solvents, such as dimethyl sulphoxide, water and acidified water.

All suitable carriers may in principle be used for compositions described herein, in different aspects. Suitable carriers are in particular: for example, ammonium salts and ground natural minerals such as kaolins, clays, talc, chalk, quartz, attapulgite, montmorillonite or diatomaceous earth, and ground synthetic minerals, such as finely divided silica, alumina and natural or synthetic silicates, resins, waxes and/or solid fertilizers. Mixtures of such carriers may likewise be used. Carriers suitable for granules include the following: for example, crushed and fractionated natural minerals such as calcite, marble, pumice, sepiolite, dolomite, and also synthetic granules of inorganic and organic meals, and also granules of organic material such as sawdust, paper, coconut shells, maize cobs and tobacco stalks.

Examples of emulsifiers and/or foam-formers, dispersants or wetting agents having ionic or nonionic properties, or mixtures of these surface-active substances, are salts of polyacrylic acid, salts of lignosulphonic acid, salts of phenolsulphonic acid or naphthalenesulphonic acid, polycondensates of ethylene oxide with fatty alcohols or with fatty acids or with fatty amines, with substituted phenols (preferably alkylphenols or arylphenols), salts of sulphosuccinic esters, taurine derivatives (preferably alkyltaurates), phosphoric esters of polyethoxylated alcohols or phenols, fatty acid esters of polyols, and derivatives of the compounds containing sulphates, sulphonates and phosphates, examples being alkylaryl polyglycol ethers, alkylsulphonates, alkyl sulphates, arylsulphonates, protein hydrolysates, lignin-sulphite waste liquors and methylcellulose. The presence of a surface-active substance is advantageous if one of the active compounds and/or one of the inert carriers is not soluble in water and if application takes place in water. Preferred emulsifiers are alkylaryl polyglycol ethers.

Further auxiliaries that may be present in the formulations and in the application forms derived from them include colorants such as inorganic pigments, examples being iron oxide, titanium oxide, Prussian Blue, and organic dyes, such as alizarin dyes, azo dyes and metal phthalocyanine dyes, and nutrients and trace nutrients, such as salts of iron, manganese, boron, copper, cobalt, molybdenum and zinc.

Stabilizers, such as low-temperature stabilizers, preservatives, antioxidants, light stabilizers or other agents which improve chemical and/or physical stability may also be present. Additionally present may be foam-formers or defoamers.

As used herein, the term “about” is defined as plus or minus ten percent of a recited value. For example, about 1.0 g means 0.9 g to 1.1 g.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicates otherwise.

It will be obvious to those skilled in the art that such embodiments are provided by way of example only. Numerous variations, changes, and substitutions will occur to those skilled in the art without departing from the embodiments of the claims. Various alternatives to the embodiments of the claims described herein may be employed in practicing the use of compositions and methods of treatment described herein. It is intended that the included claims define the scope of the various compositions and methods of treatment described herein and that methods and structures within the scope of these claims and their equivalents are covered thereby. All publications, patents, and patent applications mentioned in this specification are herein incorporated by reference to the same extent as if each individual publication, patent, or patent application was specifically and individually indicated to be incorporated by reference.

The compositions and formulations described herein are useful as biopesticide compositions in the treatment, amelioration and/or prevention of the pathological conditions as described herein in a plant in need thereof. The term “treatment” refers to both therapeutic treatment and prophylactic or preventative measures. Treatment includes the application or administration of the composition preferably in a formulation to the body, including plant leaves, shoots, roots or seeds, an isolated tissue, or cell from a plant which has a disease/disorder, a symptom of a disease/disorder, or a predisposition toward a disease/disorder, with the purpose to cure, heal, alleviate, relieve, alter, remedy, ameliorate, improve, or affect the disease, the symptom of the disease, or the predisposition toward the disease.

The term “amelioration” as used herein refers to any improvement or treatment of the disease state of a plant having an infection or other pathological condition as specified herein, by the administration of a peptide construct according to the compositions and methods described herein to a plant subject in need thereof. Such an improvement may also be seen as a slowing, arresting or stopping of the progression of an infection in the plant. The term “prevention” as used herein means compositions and methods described herein for prevention or to protect the plant, the avoidance of the occurrence or re-occurrence of a plant having an infection as specified herein below, by the administration of a peptide composition according to the compositions and methods of treatment described herein to a subject in need thereof.

The term disease resistance refers to the ability to prevent or reduce the presence of a disease or diseases in an otherwise susceptible host.

The term “disease” refers to any condition that would benefit from treatment with the peptide(s) construct or the formulated composition described herein. This includes chronic and acute disorders or diseases including those pathologic.

The term “residue” refers to an amino-acid residue. When two or more amino acids combine to form a peptide, the elements of water are removed, and what remains of each amino acid is called an amino-acid residue (two residues for example when two amino acids combine to form a peptide).

The term “amino acid” refers to organic compounds that contain both amino and carboxylic acid functional groups. Over 500 amino acids exist in nature, a larger number of synthetic amino acids are known. 22 alpha amino acids appear in the genetic code. Amino acids can be classified according to the locations of the core structural functional groups, as alpha-(α-), beta-(β-), gamma-(γ-) or delta-(δ-) amino acids; other categories relate to polarity, ionization, and side chain group type (aliphatic, acyclic, aromatic, containing hydroxyl or sulfur, etc.) described in the art.

An “amino acid” can be represented using any of the one letter or three letter symbols set forth in this specification and commonly used in the art of chemistry and biology. Examples of amino acids include L-alanine, L-arginine, L-asparagine, L-aspartic acid, L-cysteine, L-glutamine, L-glutamic acid, L-glycine, L-histidine, L-isoleucine, L-leucine, L-lysine, L-methionine, L-phenylalanine, L-proline, L-pyrrolysine, L-serine, L-selenocysteine, L-threonine, L-tryptophan, Ltyrosine, or L-valine. Amino acids can include, inter alia, D-amino acids and amino acids containing modified or synthetic side chains.

A peptide is a chain of amino acid residues. The amino acid residues in a peptide are connected to one another in a sequence by bonds called peptide bonds. When two or more amino acids combine to form a peptide, the elements of water are removed, and what remains of each amino acid is called an amino-acid residue. The amino acid residues or “residue” in a peptide are identified by one letter or three letter symbols commonly known in the art.

Sequences are described by listing, in order, each residue of the sequence, wherein: (i) the residue is represented by a name, abbreviation, symbol, or structure (e.g., HHHHHHQ or HisHisHisHisHisHisGln).

A “sequence identification number” abbreviated herein as “SEQ ID NO:” means a unique number (integer) assigned to each peptide sequence described herein and included in the attached sequence listing.

“Percentage of sequence identity” refers to comparisons among polypeptide or polynucleotides sequences, and is determined by comparing two optimally aligned sequences over a comparison window, wherein the portion of the polypeptide or polynucleotides sequence in the comparison window may comprise substitutions, additions or deletions (i.e., gaps) as compared to the reference sequence (which does not comprise substitutions, additions or deletions) for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. For example, for 20mer peptide sequence LYCNVGSHMECVKHQCKCIK (SEQ ID NO: 11), if a peptide named “11a” in which the first amino acid leucine residue “L” is replaced with a glycine “G” amino acid residue, 11a will have the sequence GYCNVGSHMECVKHQCKCIK (example new SEQ ID 11a) with 19 residues matching sequence SEQ ID NO:11. Thus, peptide 11a has a 95% sequence identity to SEQ ID NO: 11 peptide (19 matching residues/20 total residues in reference sequence=0.95, multiplied by 100=95% sequence identity).

In some aspects, an antibacterial composition comprises a peptide with an amino acid sequence having at least 85%, at least 90% or at least 95%, amino acid sequence identity to the amino acid sequence provided in peptide of SEQ ID No. 8

In some aspects, an antibacterial composition comprises a peptide with an amino acid sequence having at least 85%, at least 90% or at least 95%, amino acid sequence identity to the amino acid sequence provided in peptide of SEQ ID No. 9

In some aspects, an antibacterial composition comprises a peptide with an amino acid sequence having at least 85%, at least 90% or at least 95%, amino acid sequence identity to the amino acid sequence provided in peptide of SEQ ID No. 10

In some aspects, an antibacterial composition comprises a peptide with an amino acid sequence having at least 85%, at least 90% or at least 95%, amino acid sequence identity to the amino acid sequence provided in peptide of SEQ ID No. 11

In some aspects, an antibacterial composition comprises a peptide with an amino acid sequence having at least 85%, at least 90% or at least 95%, amino acid sequence identity to the amino acid sequence provided in peptide of SEQ ID No. 14

The term N-terminal acetylated refers to the covalent attachment of an acetyl group (CH3CO) to the free α-amino group (NH3+) at the N-terminal end of a peptide chain forming an CH3CONH-functional group on the α-amino group of the N-terminal residue of the peptide. An acetylated residue is an CH3CONH-functional group unspecified in its position on a peptide chain formed by acetylation of a free amino functional group on any of the residues of a peptide.

The terms disulfide bridge and cyclization refers to an S—S bond or a disulfide bridge and is usually derived by the coupling of two thiol (—SH) groups. A disulfide bond is formed for example when a sulfur atom from one cysteine residue in a peptide chain forms a single covalent bond with another sulfur atom from a second cysteine residue (two —SH groups react to form a —S—S— disulfide) located on the peptide chain. Disulfide bond bridges are known to help stabilize peptides.

The term amidated CONH2 C-terminus residue is the conversion of the C-terminal amino acid residue carboxylic acid functional group to a carboxamide function (Pep-NH—CHR—COOH converted to Pep-NH—CHR—CONH2). Peptide sequences bearing amidated CONH2 C-terminus residues are listed herein with an NH2 group added to the end of the sequence for example amidated SEQ ID NO: 8 is YSSCATKEECKCPDNKRPACNH2. Other sequences bearing amidated CONH2 C-terminus residues are also described herein by the sequence number of the peptide and language indicating the presence of the amidated CONH2 C-terminus residue at the C-terminus of the molecule. Hence, NH2 indicates the amidated C-terminus with the conversion of the C-terminal amino acid residue carboxylic acid functional group to a carboxamide function (Pep-NH—CHR—COOH converted to Pep-NH—CHR—CONH2).

Exemplified amidated CONH2 C-terminus compositions are denoted and described in the following examples as—amidated SEQ ID NO: 8; the sequence of which is YSSCATKEECKCPDNKRPACNH2; amidated SEQ ID NO: 9; the sequence of which is RGCKRDKDCPQFRGVNIRCRNH2; amidated SEQ ID NO: 10; the sequence of which is VKCVLPRIARCIKYRCQCRNNH2; amidated SEQ ID NO: 11; the sequence of which is LYCNVGSHMECVKHQCKCIKNH2; amidated SEQ ID NO: 14; the sequence of which is QNLCVGSPLPLQCLKFICRCNH2. Any of the peptides with SEQ ID NO 1 to SEQ ID NO 623 listed herein can have an amidated CONH2 C-terminus. The amidated CONH2 C-terminus compositions listed above are useful in methods of treatment of citrus greening disease.

The term antibacterial describes a substance that kills, stops, or reduces the growth of bacteria growing and causing disease.

The term pesticide describes any substance or mixture of substances intended for preventing, destroying, repelling, or mitigating any pest.

### Molecular Biological Methods to Express Plant-Derived Active Peptides in Cells

In some embodiments, the plant-derived active peptides disclosed herein are produced from a recombinant DNA alone or fused to a linker sequence, a 2A sequence for a self-cleaving peptide, other peptides, a reporter protein, export signals, membrane targeting sequences, sub-cellular targeting signals, signal peptides or other polynucleotides of interest for expression in microbes, viruses, plant and other eukaryotic cells.

Further, the plant-derived active peptide sequences may be delivered to plants genetically through the use of transgenic plants, viral vectors, synthetic microbes, modified plant cells expressing a gene or genes of interest together with plant growth regulator genes to initiate autonomous cell division referred to in one embodiment as a symbiont; U.S. patent application Ser. No. 11/228,659 incorporated herein by reference, U.S. patent application Ser. No. 10/465,008 incorporated herein by reference, U.S. patent application Ser. No. 18/295,882 incorporated herein by reference, U.S. patent application Ser. No. 12/705,845 incorporated herein by reference, U.S. patent application U.S. Ser. No. 17/635,494 incorporated by reference herein in their entirety or other means known to those skilled in the art of molecular biology when the protein sequence is encoded in a plasmid, vector, virus, or viral vector as a recombinant nucleic acid DNA or RNA sequence either alone or in combination with other peptides, signal peptides or as fusion to other biomolecules. U.S. Pat. No. 10,851,381; Dawson et al is incorporated by reference herein in its entirety.

An isolated nucleic acid is a nucleic acid the structure of which is not identical to that of any naturally occurring nucleic acid. The term therefore covers, for example, (a) a DNA which has the sequence of part of a naturally occurring genomic DNA molecule but is not flanked by both of the coding or noncoding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in mixtures of (i) DNA molecules, (ii) transformed or transfected cells, and (iii) cell clones, e.g., as these occur in a DNA library such as a cDNA or genomic DNA library.

The term “recombinant nucleic acids” refers to polynucleotides which are made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

In practicing some embodiments of the disclosure disclosed herein, it can be useful to modify the genomic DNA, chloroplast DNA or mitochondrial DNA of a recombinant strain of a host cell to produce plant-derived active peptide sequences, or mutant thereof to introduce genetic elements allowing for the expression of introduced genes (e.g., promoters and other regulatory elements). In some embodiments, such a host cell is a plant cell. In preferred embodiments, the host cell is a citrus plant cell.

Modifications intended to alter function of a target protein can involve mutations of the DNA or gene encoding the target protein, including deletion of all or a portion of a target gene, including but not limited to the open reading frame of a target locus. Such deletional mutations can be achieved using any technique known to those of skill in the art. Mutational, insertional, and deletional variants of the disclosed nucleotide sequences and genes can be readily prepared by methods which are well known to those skilled in the art. It is well within the skill of a person trained in this art to make mutational, insertional, and deletional mutations which are equivalent in function to the specific ones disclosed herein.

Where a recombinant nucleic acid is intended for expression, cloning, or replication of a particular sequence, DNA constructs prepared for introduction into a host cell will typically comprise a replication system (i.e. vector) recognized by the host, including the intended DNA fragment encoding a desired polypeptide, and can also include transcription and translational initiation regulatory sequences operably linked to the polypeptide-encoding segment. Additionally, such constructs can include cellular localization signals (e.g., chloroplast localization signals). In preferred embodiments, such DNA constructs are introduced into a citrus plant host cell's genomic DNA, chloroplast DNA or mitochondrial DNA.

In some embodiments, a non-integrated expression system can be used to induce expression of one or more introduced genes. Expression systems (expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides can also be included where appropriate from secreted polypeptides of the same or related species, which allow the protein to cross and/or lodge in cell membranes, cell wall, or be secreted from the cell.

Selectable markers useful in practicing the methodologies of the disclosure disclosed herein can be positive selectable markers. Typically, positive selection refers to the case in which a genetically altered cell can survive in the presence of a toxic substance only if the recombinant polynucleotide of interest is present within the cell. Negative selectable markers and screenable markers are also well known in the art and are contemplated by the present disclosure. One of skill in the art will recognize that any relevant markers available can be utilized in practicing the inventions disclosed herein.

Screening and molecular analysis of recombinant organisms (e.g., transgenic plants or recombinant bacteria) of the present disclosure can be performed utilizing nucleic acid hybridization techniques. Hybridization procedures are useful for identifying polynucleotides, such as those modified using the techniques described herein, with sufficient homology to the subject regulatory sequences to be useful as taught herein. The particular hybridization techniques are not essential to the subject disclosure. As improvements are made in hybridization techniques, they can be readily applied by one of skill in the art. Hybridization probes can be labeled with any appropriate label known to those of skill in the art. Hybridization conditions and washing conditions, for example temperature and salt concentration, can be altered to change the stringency of the detection threshold. See, e.g., Sambrook et al. (1989) vide infra or Ausubel et al. (1995) Current Protocols in Molecular Biology, John Wiley & Sons, NY, N.Y., which is incorporated herein by reference for further guidance on hybridization conditions.

Additionally, screening and molecular analysis of genetically altered strains, as well as creation of desired isolated nucleic acids can be performed using Polymerase Chain Reaction (PCR). PCR is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. (1985) Science 230:1350-1354) incorporated herein by reference. PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Because the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes which can be used are known to those skilled in the art.

Nucleic acids and peptides of the present disclosure can also encompass homologues of the specifically disclosed sequences. Homology (e.g., sequence identity) can be 50%-100%. In some instances, such homology is greater than 80%, greater than 85%, greater than 90%, or greater than 95%. The degree of homology or identity needed for any intended use of the sequence(s) is readily identified by one of skill in the art. As used herein percent sequence identity of two nucleic acids is determined using an algorithm known in the art, such as that disclosed by Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:402-410. BLAST nucleotide searches are performed with the NBLAST program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (NBLAST and XBLAST) are used. See www.ncbi.nih.gov.

Recombinant host cells (such as transgenic plant cells or recombinant microbial cells), in the present context, are those which have been genetically modified to contain an isolated nucleic acid molecule, and/or contain one or more genes to produce at least one recombinant protein. The nucleic acid(s) encoding the peptides or protein(s) of the present disclosure can be introduced by any means known to the art which is appropriate for the particular type of cell, including without limitation, transformation, lipofection, electroporation or any other methodology known by those skilled in the art.

### Transgenic Plants and Plant Cells

One embodiment of the present disclosure provides a plant or plant cell comprising one or more introduced genes encoding for plant-derived active peptide sequences, or modified plant-derived active peptide sequences. For example, the present disclosure provides transgenic plants that express plant-derived active peptide sequences, plant-derived active peptide sequences variants and other modified versions thereof, including those toxic to psyllids, those which decrease CLas transmission, mitigate citrus greening disease and protect plants against citrus greening disease.

Transformation and generation of genetically altered monocotyledonous and dicotyledonous plant cells is well known in the art. Sec, e.g., Weising, et al., Ann. Rev. Genet. 22:421-477 (1988); U.S. Pat. No. 5,679,558; Agrobacterium Protocols, ed: Gartland, Humana Press Inc. (1995); and Wang, et al. Act a Hort. 461:401-408 (1998), all incorporated herein by reference. A number of alternative techniques can also be used for inserting DNA into a host plant cell. Those techniques include, but are not limited to, transformation with T-DNA delivered by Agrobacterium tumefaciens or Agrobacterium rhizogenes as the transformation agent. Plants may be transformed using Agrobacterium technology, as described, for example, in U.S. Pat. Nos. 4,605,627; 5,177,010; 5,104,310; 5,977,441; European Patent Application No. 0131624B1, European Patent Application No. 120516, European Patent Application No. 159418B1, European Patent Application No. 176112, U.S. Pat. Nos. 5,149,645, 5,469,976, 5,464,763, 4,940,838, 4,693,976, European Patent Application No. 116718, European Patent Application No. 290799, European Patent Application No. 320500, European Patent Application No. 604662, European Patent Application No. 627752, European Patent Application No. 0267159, European Patent Application No. 0292435, U.S. Pat. Nos. 5,231,019, 463,174, 4,762,785, 5,004,863, and 5,159,135 all incorporated herein by reference. The use of T-DNA-containing vectors for the transformation of plant cells has been intensively researched and sufficiently described in European Patent Application 120516; An et al. (1985, Embo J. 4:277-284), Fraley et al. (1986, Crit. Rev. Plant Sci. 4:1-46), and Lee and Gelvin (2008, Plant Physiol. 146:325-332) all incorporated herein by reference, and is well established in the field. The choice of method varies with the type of plant to be transformed, the particular application and/or the desired result. The appropriate transformation technique is readily chosen by the skilled practitioner.

Any methodology known in the art to delete, insert or otherwise modify the cellular DNA (e.g., genomic DNA and organelle DNA) can be used in practicing the inventions disclosed herein. For example, a disarmed Ti-plasmid, containing a genetic construct for deletion or insertion of a target gene, in Agrobacterium tumefaciens can be used to transform a plant cell, and thereafter, a transformed plant can be regenerated from the transformed plant cell using procedures described in the art, for example, in EP 0116718, EP 0270822, PCT publication WO 84/02913 and published European Patent application (“EP”) 0242246. US patents U.S. Pat. Nos. 8,334,139B1, 5,352,605A, 6,174,724B1 are incorporated by reference herein. Ti-plasmid vectors each contain the gene between the border sequences, or at least located to the left of the right border sequence, of the T-DNA of the Ti-plasmid. Of course, other types of vectors can be used to transform the plant cell, using procedures such as symbiont technology (WO 21/055656), direct gene transfer (as described, for example in U.S. patent application U.S. Ser. No. 18/295,882 and U.S. Ser. No. 17/635,494 incorporated by reference in their entirety herein; EP 0233247), pollen mediated transformation (as described, for example in EP 0270356, PCT publication WO 85/01856, and U.S. Pat. No. 4,684,611), plant RNA virus-mediated transformation (as described, for example in EP 0 067 553 and U.S. Pat. No. 4,407,956), liposome-mediated transformation (as described, for example in U.S. Pat. No. 4,536,475), and other methods such as the methods for transforming certain lines of corn (e.g., U.S. Pat. No. 6,140,553; Fromm et al., Bio/Technology (1990) 8, 833-839); Gordon-Kamm et al., The Plant Cell, (1990) 2, 603-618) and rice (Shimamoto et al., Nature, (1989) 338, 274-276; Datta et al., Bio/Technology, (1990) 8, 736-740) and the method for transforming monocots generally (PCT publication WO 92/09696). For cotton transformation, the method described in PCT patent publication WO 00/71733 can be used. For viral vectors, the methods described in US patent publication US 2022/0002746 A1 can generally be used.

Transgenic plants of the present disclosure can be used in a conventional plant breeding scheme to produce more transgenic plants with the same characteristics, or to introduce the genetic alteration(s) in other varieties of the same or related plant species. Seeds, which are obtained from the transformed plants, can contain the genetic alteration(s) as a stable insert in chromosomal or organelle DNA. Plants comprising the genetic alteration(s) in accordance with the disclosure include plants comprising, or derived from, root stocks of plants comprising the genetic alteration(s) of the disclosure, e.g., fruit trees or ornamental plants. Hence, any non-transgenic grafted plant parts inserted on a transformed plant or plant part are included in the disclosure.

In one aspect a symbiont is provided to treat or prevent citrus greening disease wherein a polynucleotide of interest encoding for a peptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14 is expressed in the symbiont and the peptide expression product is transported into the host citrus plant.

In one aspect, a method of delivering a peptide compound of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14 to a citrus plant, comprising transplanting onto at least one site (e.g., 1, 2 or more sites) on a host plant a symbiont forming inoculum or a symbiont. Culturing the symbiont forming inoculum or symbiont at the at least one site on the host plant to form a symbiont on the host plant at the at least one site, wherein a polynucleotide of interest is expressed in the symbiont and the expression product of the polynucleotide of interest is a peptide having a sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14. The peptide or peptides made using the expression product of the polynucleotide of interest is transported into the host plant, thereby delivering the peptide to a citrus plant in need thereof. The site on the host plant is selected from any of an explant, embryo, leaf, shoot, stem, branch, kernel, car, cob, husk, stalk, epidermal tissue, apical meristem tissue, floral tissue (e.g., pollen, pistil, ovule, anther, stamen, corolla, sepal, petal, receptacle, filament, style, stigma, etc), fruit, seed, pod, capsule, cotyledon, hypocotyl, petiole, tuber, corm, root, root tip, symbiont, burl, plant food body, dormatia, extrafloral nectary, nodule, plant neoplasm or gall. A plant cell useful for producing a symbiont forming inoculum can be from any plant part, including but not limited to, a plant cell culture (callus, callus culture or suspension culture), a protoplast, seedling, explant, embryo, leaf, shoot, stem, branch, kernel, car, cob, husk, stalk, epidermal tissue, apical meristem tissue, floral tissue (e.g., pollen, pistil, ovule, anther, stamen, corolla, sepal, petal, receptacle, filament, style, stigma, etc.), fruit, seed, pod, capsule, cotyledon, hypocotyl, petiole, tuber, corm, root, root tip, symbiont, burl, plant food body, dormatia, extrafloral nectary, nodule, gall or plant neoplasm.

In one aspect, a symbiont comprising a plant cell expressing a polynucleotide encoding one or more phytohormone biosynthetic enzyme and a polynucleotide expressing a peptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14 is provided. The phytohormone biosynthetic enzyme is a cytokinin biosynthetic enzyme and/or at least one auxin biosynthetic enzyme and the plant cell of the symbiont autonomously divides.

In one aspect, the symbiont consists of more than one plant cell and forms a multi-cellular structure when formed on or transplanted onto a citrus plant. In another aspect, the symbiont bears a phytohormone biosynthetic enzyme which is from a bacterial species and/or a plant species.

The symbiont forming inoculum comprising the polynucleotide encoding a phytohormone biosynthetic enzyme and the polynucleotide expressing a peptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14, when comprised in plant cells may be in the form of a plant callus or callus culture or a suspension culture.

In another aspect, a symbiont produces peptides to treat citrus greening disease comprising a plant cell comprising and expressing a polynucleotide encoding a phytohormone biosynthetic enzyme and a polynucleotide of expressing a peptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14, wherein the phytohormone biosynthetic enzyme is at least one cytokinin biosynthetic enzyme and/or an auxin biosynthetic enzyme and the plant cell of the symbiont autonomously divides. In some embodiments, the plant cell comprises at least two plant cells (e.g., 2, 3, 4, 5, 6, 7, 8, 9, 10, 20, 30, 40, 50, 60, 70, 80, 90, 100, or more cells). A symbiont that comprises more than one cell may form a plant callus or callus culture or a suspension culture to produce the peptide. A symbiont comprising more than one plant cell may form an multi-cellular structure.

In another aspect, the phytohormone biosynthetic enzyme is an indole-3-acetamide hydrolase (iaaH) (E.C. Number: EC 3.5.1.4), amidase 1 (EC 3.5.1.4), a tryptophan 2-monooxygenase (IaaM) (EC 1.13.12.3), an indole-3-lactate synthase (EC 1.1.1.110), a L-tryptophan-pyruvate aminotransferase 1 (EC 2.6.1.99), a tryptophan aminotransferase-related protein 1 (EC 2.6.1.27), indole-3-acetaldehyde oxidase (EC 1.2.3.7), a tryptophan decarboxylase 1/tryptophan decarboxylase 2 (EC4.1.1.105), an isopentenyl transferase (Ipt) and/or a Tzs (EC 2.5.1.27).

In one aspect, the polynucleotide encoding a phytohormone biosynthetic enzyme and the polynucleotide of interest in the symbiont are comprised in a single nucleic acid construct or in two or more nucleic acid constructs (e.g., one or more expression cassettes).

A polynucleotide of interest expressing peptides with sequences described herein with a symbiont described in embodiments herein refers to a polynucleotide encoding a molecule as described herein (e.g., one or more than one polypeptide, peptide, coding RNA or non-coding RNA; e.g., a biomolecule, a bioactive molecule) for expression in the symbiont, and optionally transported from the symbiont into a host plant on which the symbiont is affixed at one or more than one site, optionally wherein when transported into the host plant, the molecule can confer a new characteristic onto the host plant without altering the genotype or genome of the host plant. In some embodiments, a polynucleotide may encode a peptide biomolecule and/or bioactive molecule and/or may encode a biosynthetic enzyme for a biomolecule and/or bioactive molecule (e.g., a polypeptide involved in the biosynthesis of a bioactive molecule) as described herein. “A polynucleotide in a symbiont may be one polynucleotide of interest (POI) or maybe two or more (e.g., 2, 3, 4, 5, 6, 7, 8, 9, 10, 11, 12, 13, 14, or 15 or more) polynucleotides of interest expressing one or more peptides and or other bioactive molecules. When two or more polynucleotides are comprised in a symbiont, the symbiont may be referred to as a “stacked” symbiont. Additionally, one or more symbionts formed on a host plant, wherein at least two of the symbionts comprise a different POI, may be referred to as “stacked symbionts”. Stacking may also comprise forming one or more symbionts on a host plant, wherein all of the symbionts comprise the same POI(s).

In some embodiments, when (i) the polynucleotide encoding a phytohormone biosynthetic enzyme and a polynucleotide sequence to express a peptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14 or other peptides described herein to treat citrus greening disease (ii) the polynucleotide encoding a phytohormone biosynthetic enzyme are comprised in at least one plant cell, the at least one plant cell may be transplanted onto at least one site (e.g., 1, 2, 3, 4, 5, 6, 7, 8, 9, 10 or more sites) on the plant. In some embodiments, the one or more cells (e.g., 1, 2, 3, 4, 5, 6, 7, 8, 9, 10, 20, 30, 40, 50, 60, 70, 80, 90, 100 or more cells) transplanted at the at least one site are cultured at the site to produce a population of plant cells comprising the polynucleotide encoding a phytohormone biosynthetic enzyme and the polynucleotide sequence of interest and form a symbiont, wherein one or more cells from the symbiont on the plant are selected to provide one or more cells comprising the polynucleotide encoding a phytohormone biosynthetic enzyme and the polynucleotide sequence of interest, thereby producing the symbiont forming inoculum.

In one aspect, a symbiont is a single plant cell that comprises at least one pSYM (SYMbiont-forming plasmid), a plasmid comprising at least one polynucleotide (or more polynucleotides) encoding one or more phytohormone biosynthetic enzymes and at least one polynucleotide(s) for synthesis of at least one of peptides with a sequence of SEQ ID NO 1 to SEQ ID NO 623 described herein or it may comprise two or more cells each of which comprises at least one pSYM, a plasmid comprising at least one polynucleotide encoding one or more phytohormone biosynthetic enzymes and at least one polynucleotide for synthesis of at least one of peptides of SEQ ID NO 1 to SEQ ID NO 623. The cells of a symbiont autonomously divide which form an undifferentiated multi-cellular structure on a plant. In some embodiments, the undifferentiated multi-cellular structure (e.g., symbiont) that is formed may be visually similar to, for example, a burl, a plant food body, a dormatia, an extrafloral nectary, a nodule, plant neoplasm or gall, but which are biochemically/genetically distinct by at least the transgenes expressed in the symbiont.

A phytohormone biosynthetic enzyme useful with a symbiont described herein and can be any auxin or cytokinin biosynthetic enzyme that can be expressed in a plant cell to produce a plant cell that autonomously divides or replicates, optionally to produce an undifferentiated multi-cellular structure. These have been described in detail above and include auxin biosynthetic enzymes that include, but are not limited to, indole-3-acetamide hydrolase (iaaH) (E.C. Number: EC 3.5.1.4), amidase 1 (EC 3.5.1.4), tryptophan 2-monooxygenase (IaaM) (EC 1.13.12.3), indole-3-lactate synthase (EC 1.1.1.110), L-tryptophan-pyruvate aminotransferase 1 (EC 2.6.1.99), tryptophan aminotransferase-related protein 1 (EC 2.6.1.27), indole-3-acetaldehyde oxidase (EC 1.2.3.7), and/or tryptophan decarboxylase 1/tryptophan decarboxylase 2 (EC4.1.1.105). In some embodiments, the phytohormone biosynthetic enzyme is a cytokinin biosynthetic enzyme that can include, but is not limited to, isopentenyl transferase (Ipt) (synonyms: adenosine phosphate-isopentenyltransferase; adenylate dimethylallyltransferase; (dimethylallyl) adenosine tRNA methylthiotransferase) (E.C. Number: 2.5.1.27 or 2.5.1.75 or 2.5.1.112) and/or Tzs (synonyms: dimethyl transferase, isopentenyl transferase, trans-zeatin producing protein, adenylate dimethylallyltransferase) (EC 2.5.1.27). In some embodiments, the phytohormone biosynthetic enzyme may be an indole-3-acetamide hydrolase (iaaH), a tryptophan 2-monooxygenase (IaaM), and/or an isopentenyl transferase (Ipt). In some embodiments, a symbiont may further comprise a polynucleotide encoding a phytohormone biosynthetic enzyme that is indole-3-lactate synthase.

In some embodiments, a symbiont may further comprise and express a polynucleotide encoding a plast polypeptide (e.g., plasticity polypeptide). A plast polypeptide that is useful can be any plast polypeptide now known or later discovered that can confer a benefit on the structure of a symbiont that is formed using the nucleic acid constructs. In some embodiments, a plast polypeptide may be a 6b, rolB, rolC, and/or orf13. In some embodiments, more than one polynucleotide encoding a plast polypeptide may be comprised in a symbiont.

In some embodiments, culturing a symbiont forming inoculum, when comprised in a bacterial cell on a host plant, can further comprise culturing in the presence of acetosyringone at a concentration in a range from about 10 μM to about 200 μM or any range or value therein (e.g., about 10, 15, 20, 25, 30, 350 40, 45, 50, 60, 70, 80, 90, 100, 110, 120, 130, 140, or 150μ, or any range or value therein) (e.g., about 50 μM-about 150 μM, about 75 μM to about 125 μM, about 85 μM to about 100 μM). In some embodiments, when culturing in the presence of acetosyringone, the acetosyringone is present at a concentration of about 100 μM.

In some embodiments, a symbiont forming inoculum comprising bacterial cells may be used to modify a host plant characteristic without modifying the host plant genome. In some embodiments, a symbiont forming inoculum containing Agrobacterium spp. may be delivered, for example, to a first plant. The Agrobacterium spp. may be in the form of one or multiple strains, where at least one strain contains a nucleic acid encoding at least one phytohormone biosynthetic enzyme (that may be provided, for example, in a T-DNA) that induces symbiont-formation and at least one strain contains a nucleic acid that comprises a polynucleotide for synthesis of at least one of peptides of SEQ ID NO 1 to SEQ ID NO 623 (that may be provided, for example, in a T-DNA) encoding a desired trait to be imparted to a host plant. The delivery of the inoculum can thus cause one or more symbionts to form on the first plant, and the symbionts can express the nucleic acids delivered by the Agrobacterium spp. The symbionts have increased vascularization in the symbiont tissue, which itself supports rapid growth, more rapid metabolism, and an effective pathway for export and ultimately systemic movement of desired molecules throughout the plant. In some embodiments, a symbiont may then be removed from the first plant and affixed/transplanted onto a second plant (e.g., a host plant) so as to be in functional communication with the host plant, thus forming a plant tissue which supplies the host plant with the desired trait but without transforming or altering the genome of the host plant or introducing heterogeneous or xenobiotic DNA into the host plant. In some embodiments, prior to transplantation to the host plant, the removed symbiont, now symbiont forming inoculum may be cultured without Agrobacterium spp. to form a bacteria-free symbiont forming inoculum after which the symbiont forming inoculum may be transplanted to the host plant.

Regarding the choice of Agrobacterium spp. strain(s) to be used, various single strains or combinations thereof are usable to achieve the desired results. According to one embodiment, the inoculum includes at least two strains where at least one strain used is an “activated strain” (such as a wild-type strain) that comprises at least one polynucleotide encoding a phytohormone biosynthetic enzyme, and at least one other strain is not an activated strain (e.g., “disarmed”, “trait inducing” strains) but comprises nucleic acid (e.g., T-DNA) that imparts a desired trait (polynucleotide of interest) in the host plant. The activated strain may be isolated from nature, such as the FL-F54 strain, as wild-type Agrobacterium spp. are known to form galls. The desired trait may be, for example, treatment of citrus greening disease and optionally other traits. The trait may be expressed or effected by one or more molecules (e.g., 1, 2, 3, 4, 5, 6, 7, 8, 9, 10 or more molecules), such as molecules encoded by the nucleic acid (e.g., T-DNA) in the trait-inducing Agrobacterium spp. Multiple activated strains and/or multiple trait-inducing strains may be used, as desired for a particular application. Alternatively, a single strain may be used that both induces symbiont formation and also induces a desired trait in a host plant to which the symbiont is affixed without modifying the host plant genome. The inoculum may contain one or more Agrobacterium spp. strain(s) (e.g., 1, 2, 3, 4, 5, or more strains) as described above in addition to a carrier, and other ingredients, as desired. If multiple strains are used, various ratios of strains may be used, as desired, for example, a 1:10 ratio of activated strain to trait-inducing strain. Agrobacterium spp. delivery inoculums are well-known in the art, and a suitable one can be chosen based on the desired outcome in a particular application. For example, the inoculum may contain an aqueous solution of a buffer, such as MES (2-ethanesulfonic acid), Tris (tris(hydroxymethyl)aminomethane), HEPES (4-(2-hydroxyethyl)-1-piperazineethanesulfonic acid), or a salt-based buffer such as PBS (phosphate-buffered saline); one or more salts, such as magnesium chloride, a transformation enhancer, such as acetosyringone or other phenolics that can enhance virulence and/or an adjuvant including, but not limited to, wetting/penetrating enhancing surfactant agents, including but not limited to anionic, cationic, and nonionic surfactants. The delivery of the inoculum may be achieved by any known method, such as via a needle, a puncture wound, or other direct delivery systems, i.e. use of drilling or air blasting, and may be automated or manual.

Symbiont formation can be observed by eye, and symbiont size can optionally be controlled via known means, such as chemical control (i.e. GALLEX® (AgBioChem Inc., Los Molinos, Calif.)). Symbiont formation may take various amounts of time, depending on the host plant species and the age of the plant used. For example, sufficient symbiont formation may take several days to several months to develop. In some embodiments, a symbiont or symbiont tissue can be collected from a first plant and then cultured for increased volume or storage purposes. In some embodiments, a symbiont may be moved directly from a first plant to a second plant (e.g., host plant) without culturing. However, it may be desired to culture the symbiont forming inoculum first to (a) remove residual bacteria, such as by attrition or by active sterilization, or (b) determine that the symbiont forming inoculum expresses the desired trait(s). Removal of residual Agrobacterium spp. may occur over time by attrition, such as by supplying a culture that does not support the bacteria and thus it dies off, or by active means, such as by sterilization with the application of bleach and/or antibiotics or other methods which actively kill bacterial cultures. The determination of whether the symbiont informing inoculum or a symbiont expresses a desired trait may be accomplished by simple observation, if the trait is phenotypically visible (such as a color), or by analysis of the culture medium/host plant for the target compound(s) being produced by the symbiont or symbiont forming inoculum, or by any other known means.

In some embodiments, introducing a polynucleotide (e.g., a polynucleotide encoding a phytohormone biosynthetic enzyme (e.g., at least one polynucleotide encoding at least one phytohormone biosynthetic enzyme), a polynucleotide to express one or more peptides to control citrus greening disease; expression cassette(s) or vector(s) comprising the same) into a plant cell, plant or part thereof is carried out via bacterial mediated transformation and comprises co-cultivating the plant cell or plant (or a part thereof, e.g., explant) with the cells of at least one bacterial species or strain (e.g., 1, 2, 3, 4, 5, or more), the bacterial cells comprising one or more of: the polynucleotide encoding a phytohormone biosynthetic enzyme, the polynucleotide of interest, and/or at least one polynucleotide encoding at least one plast polypeptide. In some embodiments, the plant (or part thereof; e.g., explant) may be wounded at the site of inoculation prior to or during co-cultivation with the cells of the at least one bacterial strain. In some embodiments, the cells of the at least one bacterial species or strain comprise cells of at least two bacterial species or strains and the polynucleotide encoding a phytohormone enzyme is comprised in a separate bacterial strain from the bacterial strain comprising the at least one polynucleotide to express a peptide(s) to control citrus greening disease (e.g., dual bacterial transformation). As described herein, a bacterial cell useful for producing a symbiont forming inoculum may be any bacterial cell comprising a Type IV Secretion System (T4SS, e.g., T4ASS, (e.g., VirB/D4 system), T4BSS) or a Type III Secretion System (T3SS), and can include, but are not limited to, those of Agrobacterium spp. (e.g., A. tumefaciens (e.g., biovar 1), A. rhizogenes (e.g., biovar 2), A. vitis (e.g., biovar 3), A. fabrum (e.g., strain C58), Rhizobium spp., Mesorhizobium spp., Sinorhizobium spp., Bradyrhizobium spp., Pseudomonas spp., Phyllobacterium spp., Ochrobactrum spp., Azobacter spp., Closterium spp., Klebsiella spp., Rhodospirillum spp., or Xanthomonas spp. In some embodiments, a Pseudomonas spp. (e.g., P. savastanoi pv. Savastanoi). In some embodiments, a bacterial cell may be a Pseudomonas savastanoi pv. Savastanoi cell. The plant species to which this method may be applied is not limited. As discussed above, since at least the early 1980's, through human intervention, the ability of bacteria to transfer DNA to plants has been extended to many species, beyond those that are naturally infected by the bacteria.

Introduced genetic elements, whether in an expression vector or expression cassette, which result in the expression of an introduced gene will typically utilize a plant-expressible promoter. A ‘plant-expressible promoter’ as used herein refers to a promoter that ensures expression of the genetic alteration(s) of the disclosure in a plant cell. Examples of promoters directing constitutive expression in plants are known in the art and include: the strong constitutive 35S promoters (the “35S promoters”) of the cauliflower mosaic virus (CaMV), e.g., of isolates CM 1841 (Gardner et al., Nucleic Acids Res, (1981) 9, 2871-2887), CabbB-S (Franck et al., Cell (1980) 21, 285-294) and CabbB-JI (Hull and Howell, Virology, (1987) 86, 482-493); promoters from the ubiquitin family (e.g., the maize ubiquitin promoter of Christensen et al., Plant Mol Biol, (1992) 18, 675-689), the gos2 promoter (de Pater et al., The Plant J (1992) 2, 834-844), the emu promoter (Last et al., Theor Appl Genet, (1990) 81, 581-588), actin promoters such as the promoter described by An et al. (The Plant J, (1996) 10, 107), the rice actin promoter described by Zhang et al. (The Plant Cell, (1991) 3, 1155-1165); promoters of the Cassava vein mosaic virus (WO 97/48819, Verdaguer et al. (Plant Mol Biol, (1998) 37, 1055-1067), the pPLEX series of promoters from Subterranean Clover Stunt Virus (WO 96/06932, particularly the S4 or S7 promoter), an alcohol dehydrogenase promoter, e.g., pAdh1S (GenBank accession numbers X04049, X00581), and the TR1′ promoter and the TR2′ promoter (the “TR1′ promoter” and “TR2′ promoter”, respectively) which drive the expression of the l′ and 2′ genes, respectively, of the T-DNA (Velten et al., EMBO J, (1984) 3, 2723-2730).

Alternatively, a plant-expressible promoter can be a tissue-specific promoter, i.e., a promoter directing a higher level of expression in some cells or tissues of the plant, e.g., in green tissues (such as the promoter of the PEP carboxylase). The plant PEP carboxylase promoter (Pathirana et al., Plant J, (1997) 12:293-304) has been described to be a strong promoter for expression in vascular tissue and is useful in one embodiment of the current disclosure. Alternatively, a plant-expressible promoter can also be a wound-inducible promoter, such as the promoter of the pea cell wall invertase gene (Zhang et al., Plant Physiol, (1996) 112:1111-1117). A ‘wound-inducible’ promoter as used herein means that upon wounding of the plant, cither mechanically or by insect feeding, expression of the coding sequence under control of the promoter is significantly increased in such plant. These plant-expressible promoters can be combined with enhancer elements, they can be combined with minimal promoter elements, or can comprise repeated elements to ensure the expression profile desired.

In some embodiments, genetic elements can be used to increase expression in plant cells can be utilized. For example, an intron at the 5′ end or 3′ end of an introduced gene, or in the coding sequence of the introduced gene, e.g., the hsp70 intron. Other such genetic elements can include, but are not limited to, promoter enhancer elements, duplicated or triplicated promoter regions, 5′ leader sequences different from another transgene or different from an endogenous (plant host) gene leader sequence, 3′ trailer sequences different from another transgene used in the same plant or different from an endogenous (plant host) trailer sequence.

An introduced gene of the present disclosure can be inserted in host cell DNA so that the inserted gene part is upstream (i.e., 5′) of suitable 3′ end transcription regulation signals (i.e., transcript formation and polyadenylation signals). This is preferably accomplished by inserting the gene in the plant cell genome (nuclear or chloroplast). Preferred polyadenylation and transcript formation signals include those of the nopaline synthase gene (Depicker et al., J. Molec Appl Gen, (1982) 1, 561-573), the octopine synthase gene (Gielen et al., EMBO J, (1984) 3:835-845), the SCSV or the Malic enzyme terminators (Schunmann et al., Plant Funct Biol, (2003) 30:453-460), and the T-DNA gene 7 (Velten and Schell, Nucleic Acids Res, (1985) 13, 6981-6998), which act as 3′-untranslated DNA sequences in transformed plant cells.

### EXAMPLES

Having now generally described the compositions, methods of treatment and other embodiments described herein, the same will be better understood by reference to certain specific examples, which are included herein only to further illustrate the embodiments and are not intended to limit the scope of the same as defined by the claims.

These exemplified peptide sequences were discovered by treating plants and insects infected with CLas, a causative bacterium of citrus greening disease. To conduct these experiments, CLas-infected plants and D. citri are needed. D. citri Kuwayama (Hemiptera: Liviidae) were reared in controlled growth chambers in Ithaca, NY on Citrus medica (citron) plants under 14:10 h light:dark cycle at 28 C. The colony-supporting citrus plants were regularly monitored by lab technicians, maintained by pruning and watered on a weekly or as needed basis. Individual D. citri nymphs from citron colonies were manually removed using a fine paintbrush prior to transfer to CLas infected excised leaves for the excised leaf acquisition assay as described in (Igwe et al. Phytopathology (2021) 112:69-75). CLas-infected citron plants reared separately from healthy citron under the same photoperiod and growth conditions. Infected plants were generated using D. citri inoculation and monitored for HLB development by periodic analysis of CLas DNA using qPCR and observation of symptom development.

To compute the 20-mer plant-derived active peptide sequences from the Medicago truncatula genome for peptide synthesis, a database of 662 M. truncatula nodule-specific cysteine rich (NCR) protein sequences was compiled by searching the annotated M. truncatula proteome in UniProt Knowledgebase (UniProtKB) using the keyword searches (e.g. NCR, nodule cysteine-rich). The Random Forest algorithm antimicrobial peptide (AMP) computation tool on CAMPR3 (http://www.camp.bicnirrh.res.in/predict_c/) was used to identify 20-mer sequences with antimicrobial characteristics within each of these 662 proteins (doi: 10.1093/nar/gkp1021). Out of these 662 proteins, at least one 20mer sequence with an AMP score >0.5 was identified for 623 proteins. The grand average of hydropathicity index (GRAVY) score for each peptide was calculated using the method of Kyte and Doolittle (1982). Briefly, the GRAVY score calculates the sum of the hydropathy values of all the amino acids in the peptide divided by the sequence length (Kyte and Doolittle 1982). The GRAVY score was calculated for the peptide with the highest AMP score for each of the 623 proteins for which at least one peptide had an AMP score greater than 0.5 (https://www.gravy-calculator.de/index.php). Negative peptide GRAVY scores typically suggest that peptides will be water-soluble, which is a desirable characteristic for facilitating delivery to plants and for testing in cells. However, some known antimicrobial peptides have positive GRAVY scores, so a range of GRAVY scores were considered. A total of 183 peptides with a range of GRAVY scores (163 negative, 20 positive) were selected for synthesis. Any secretion signals, which are not required for antimicrobial activity, were removed prior to synthesis (Tiricz et al. 2013b). Sequences were sent to Biomatik for synthesis.

To measure the effects of plant-derived peptides on bacterial growth, in vitro growth assays were used using Liberibacter crescens strain BT-1. Bacterial culture assays growth of Liberibacter crescens strain BT-1 was performed in BM7 basal salts (BM7) medium. Briefly, a BM7 basal salts solution was prepared by combining and dissolving 2 g alpha ketoglutaric acid sodium salt, 10 g ACES buffer, and 3.75 g potassium hydroxide in distilled water, tuning the pH to 6.9, and adjusting the final volume to 550 ml. After autoclaving and cooling to RT, fetal bovine serum (FBS, 150 ml) and TMN-FH medium (300 ml) were added aseptically to the BM7 basal salts solution by stirring. Agar medium was prepared similarly, except that 15 g of microbiological grade agar was added to the basal salts solution prior to autoclaving, and FBS and TMN-FH medium were pre-warmed in a water bath and added at 50° C. instead of RT. Prior to preparation of growth assays, L. crescens strain BT-1 was streak plated from glycerol freezer stocks onto sterile BM7 basal salts (hereafter BM7) agar medium and allowed to grow at 28° C. for 7 to 14 days, at which point a single colony was picked and inoculated into 3.5 ml BM7 broth and grown at 28° C. with shaking at 200 rpm. An aliquot of this culture (5% v/v inoculum) was transferred to fresh BM7 broth and allowed to grow to an O.D. 600 nm of 0.4 to 0.7 (roughly 3 to 4 days). The transfer culture was then diluted to an O.D. 600 nm of 0.025, and 50 microliters of diluted culture were combined in a sterile low-bind, round bottom polypropylene plate with 50 microliters of plant-derived active peptide sequences (2 or 0.2 mg/ml) diluted in BM7 medium. The final concentrations of plant-derived active peptides in all assays were 1 or 0.1 mg/ml. The antimicrobial peptide polymyxin B sulfate (0.5 mg/ml final concentration) dissolved in BM7 broth, L. crescens strain BT-1 cells in BM7 broth, and BM7 broth without cells were utilized in every 96-well plate assay as positive growth inhibition, no growth inhibition, and no growth controls, respectively. The grown inhibition of all plant-derived active peptides examined were loaded in at least duplicate technical replicates. Following their preparation, 96-well plates were wrapped with parafilm, loaded into a small plastic container lined with moist paper towels, and incubated at 28° C. with shaking (200 rpm) over a seven day period. The O.D. 600 nm was recorded daily on a Synergy HT plate reader (Agilent Technologies, Inc., Santa Clara, CA, USA).

Next, tests for whether the bioactive peptides were mobile in the plant phloem and could act as a biopesticide against CLas in citrus plants were conducted. An excised leaf assay was used to measure the impact of the obtained plant-derived active peptide sequences in CLas-infected, citrus leaves. The use of excised leaves enables measurement of systemic movement of plant-derived active peptides in the leaf vascular tissue using small amounts of peptide starting material and an assessment of phytotoxicity. Mature leaves that have been previously screened for CLas infection using a CLas qPCR assay of a small leaf disc were dissected from a citrus plant with the petiole intact. The petiole was then submerged in solutions of 0.1 mM potassium phosphate buffer, pH 5.8, (hereafter KPO4 buffer) containing either 1 mg/ml polymyxin B sulfate (PMB), or 1 mg/ml plant-derived active peptides. All leaves (n=10 per treatment) and solutions were housed in a 0.2 ml sterile PCR tube and wrapped with parafilm to prevent evaporation of the solution. After an overnight incubation period in which most of the solution was taken up by the leaf, the PCR tube with leaf was inverted, flicked gently to clear any liquid from the tip of the PCR tube, and the PCR tube cut with 70% (v/v) ethanol-wiped scissors. The leaf and PCR tube were then placed into a sterile 2 ml microcentrifuge tube containing KPO4 buffer where they remained for 7 days (FIG. 10). The KPO4 buffer was topped off as needed, usually on a daily basis and leaves were maintained in a 70° F. chamber with 14 h light: 10 h dark schedule. A set of four leaf discs (˜ 1 mm) were extracted at 0 and 7 days post access to all treatment solutions, added to a 2 ml microcentrifuge, and snap frozen in liquid nitrogen. All samples were stored at −80° C. until nucleic acid extractions were performed.

CLas acquisition by D. citri is a required step for tree-to-tree transmission of CLas, which results in the spread of citrus greening disease. We assayed whether the plant-derived active peptides could block CLas acquisition by D. citri. An excised leaf assay described in (Igwe et al. Phytopathology (2021) 112:69-75) was used to measure the effects of the plant-derived active peptides on CLas acquisition by D. citri. Evaluation of CLas titer using one punch of leaf from each of the CLas-infected colonies helped to determine which ones to use in the excised leaf assays, acknowledging variability within a plant for CLas titer at a given time point during infection precludes precise estimation of whole tree titer. The CLas-infected plants used for this assay had no psyllids on them at the time of the sample collection for DNA extraction and quantitative (q) PCR analysis. Plants with Cq<30 were used to supply the excised leaves for the analysis. Leaves from CLas-infected plants in the Heck lab inventory designated as E2, T and Q were selected, with Cq values of 26.11, 27.37 and 28.02, respectively. Stems from these CLas-infected citron plants were collected and cut at 45° C. angle with some portion of the stem still intact to facilitate water absorption via the exposed cut surface area of the citron stem. Cuttings were transferred to 0.6 mL tubes containing 200 μL plant-derived active peptides (SEQ ID NO: 9 (803543) and SEQ ID NO: 8 (803570), 20 mg/ml) diluted with KPO4 buffer to 1 mg/mL, and PMB (0.5 mg/mL) in KPO4 buffer. All cuttings were incubated for 24 hours to allow adequate uptake of the peptides, PMB and KPO4 buffer. Excised leaves in 0.6 mL containing the solutions were then transferred to 2.0 mL tubes, wrapped with parafilm, and placed inside 50 mL falcon tubes. The prepared CLas-infected citrus leaves from plants E2, T and Q were distributed equally among the treatments. The lids of the falcon tubes were subsequently replaced with hand-made ones containing mesh to enhance ventilation. Healthy psyllid nymphs (CLas-free) of 2nd and 3rd instars were painstakingly collected from established healthy citron colonies and transferred to a transparent plastic tray to remove accumulated honeydew using a fine-paint brush, followed by starvation for two hours before transferring psyllid nymphs to each treatment including the PMB and KPO4 buffer as controls. Each treatment was comprised of ten biological replicates with 10 psyllid nymphs per excised leaf. The assay buffers were monitored and buffer volume maintained by replacement with fresh KPO4 buffer for 21 days, after which the adults psyllids were collected for DNA extraction and qPCR analysis.

The bioassays mentioned above in paragraphs 193 and 192 were analyzed using qPCR to measure CLas titer in the leaves and insects after the experiment. To measure impact of different plant-derived active peptide sequences in bacterial growth and titer in citrus leaves and D. citri insects, DNA and RNA extractions were used together with qPCR. Total nucleic acids were extracted from individual adult psyllid or citrus leaves (three punches per leaf pooled prior to DNA extraction). All tissues were transferred to sterile 2 mL microcentrifuge tubes containing three sterile 3.2 mm stainless steel balls, flash frozen in liquid nitrogen and homogenized for three minutes at 25 Hz using a Laboratory Mixer Mill MM 400 (Retsch USA, Newtown, PA, USA). Following homogenization, tissue homogenates were kept on ice and diluted in 600 μL of RLT buffer (QIAGEN Sciences, Inc., Germantown, MD, USA), vortexed briefly, and then centrifuged for 5 min at 12,000 rpm. After centrifugation 450 μL of 70% (v/v) EtOH was added to an EconoSpin Mini Spin column (Epoch Life Science, Inc., Sugar Land, TX, USA) followed by an equal volume (450 μL) of tissue homogenate, which were briefly pipetted up and down to combine. The mixture was then centrifuged for one min at 8,000 rpm and the flow through discarded. Washing was performed twice for whole psyllids and three times for the leaf punches by adding 700 μL of 75% (v/v) EtOH, centrifuging for 1 min at 8,000 rpm and discarding the flow through. The EconoSpin Mini Spin column membrane was dried by additional centrifugation for two min at 12,000 rpm. The EconoSpin columns were then transferred to new 1.5 mL tubes, followed by addition of 25 or 100 μL of pre-warmed (37° C.) molecular grade water to EconoSpin columns containing psyllid and leaf nucleic acids, respectively. All columns were incubated for two min at room temperature followed by centrifugation for two min at 12,000 rpm to clute the nucleic acids. All nucleic acid extracts were quantified using a NanoDrop 2000 spectrophotometer (Thermo Fisher Scientific, Inc., Waltham, MA, USA). All samples were stored at −80° C. until further analysis.

For the detection of CLas rDNA, qPCR assays were performed on an Applied Biosystems QuantStudio 6 Flex Real-Time PCR System (Thermo Fisher Scientific, Waltham, MA, USA). The TaqMan Universal PCR master mix (Thermo Fisher Scientific) with 16S rRNA gene primer and probe sets (Ramsey et al., 2015; Saeed et al., 2019) were used. Briefly, the CLas 16S rDNA gene primers used were CLas16SF (5′-TCGAGCGCGTATGCAATACG-3′), CLas16SR (5′-GCGTTATCCCGTAGAAAAAGGTAG-3′), and probe CLas 16Sp (5′-AGACGGGTGAGTAACGCG-3′). CLas titers in all samples were quantified in duplicate for each biological replicate. The final concentration of qPCR mix contained 1×TaqMan Universal PCR master mix (10 μL) (Thermo Fisher Scientific, Inc.), 1 μL of each of forward and reverse primers (10 μM each), and 1 μL of probe (5 μM). For analysis of leaf disks in the excised leaf analysis, 2 μL DNA was used per reaction and gene copies were computed using a standard curve. For the plant and insect samples used in the excised leaf acquisition assays, 2 μL of 25 ng/μL DNA sample (50 ng total each for leaf and insect samples), and 5 μL of nuclease free water in a total volume of 20 μL. The qPCR program consisted of 2 min incubation at 50° C. followed by 10 min incubation at 95° C. and 40 cycles at 95° C. for 15 s and 60° C. for 1 min. For the analysis of CLas 16S rRNA genes, nucleic acid extracts were split in two equal portions, one of which was subjected to DNase I° C. treatment using the TURBO DNA-free kit (Thermo Fisher Scientific, Inc.). Briefly, each reaction contained 2.5 μL of 10×DNase I buffer, 0.5 L of DNase I enzyme, and 17 μL of nucleic acid extract. DNase reactions were incubated at 37° C. for 1 hr, followed by addition of 5 μL of DNase inactivation reagent and incubation at RT for 5 min, followed by centrifugation for 5 min at 2,000×g. Following DNase I treatment, 15 μL of the the supernatant above the dense inactivation reagent was transferred to a fresh, sterile DNase/RNase-free 96-well plate for first-strand cDNA synthesis. The iScript cDNA synthesis kit (Bio-Rad Laboratories, Inc., Hercules, CA, USA) was used to generate the first-strand cDNA by combining 15 μL DNase I treated RNA with 4 μL of 5×iScript master mix reagent, 0.5 μL of iScript reverse transcriptase, and 0.5 μL of DNase/RNase-free water. Samples were vortexed and centrifuged briefly and then incubated on a MiniAmp thermal cycler (Applied Biosystems) using a thermal program consisting of 25° C. for 5 min, 46° C. for 40 min, 95° C. for 1 min, and 4° C. thereafter. All first-strand cDNA and RT-minus RNA controls were stored at −80° C. until qPCR analysis.

Non-parametric methods were used to analyze the data. To compare differences in CLas rRNA and rDNA ratios in excised leaf assays, a Wilcoxon Rank Sum Test was performed. The CLas cell equivalents and Ct values from the excised leaf assays were compared to the KPO4 buffer using the Dunnet's Test. Data were tested for normality and transformed using a Box Cox transformation if needed prior to the Dunnet's test where indicated in the results section. Conversion of Cq values to CLas cell equivalents using a standard curve during qPCR as described in (Igwe et al. Phytopathology (2021) 112:69-75).

Analysis revealed a total of 604 M. truncatula proteins annotated as NCRs in UniProt (Table 3). These peptides ranged from 16 to 924 residues in length, with the majority being 90 residues or smaller. A total of 182 smaller, 20-mer antimicrobial peptides from all three tiers were identified from the larger sequences for synthesis based on their GRAVY scores.

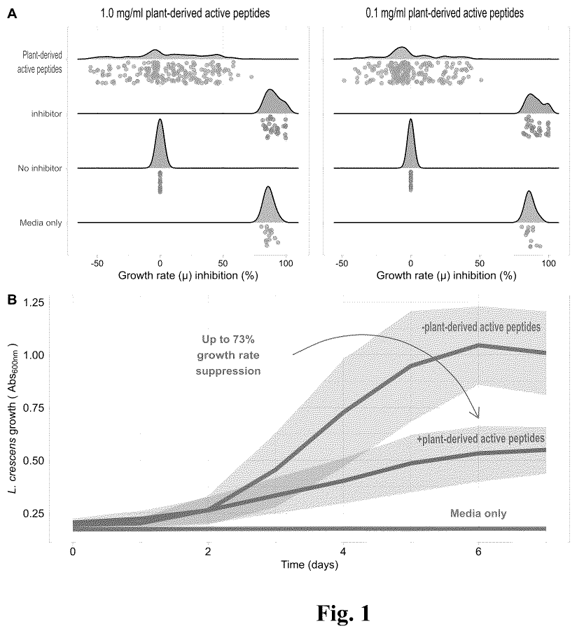

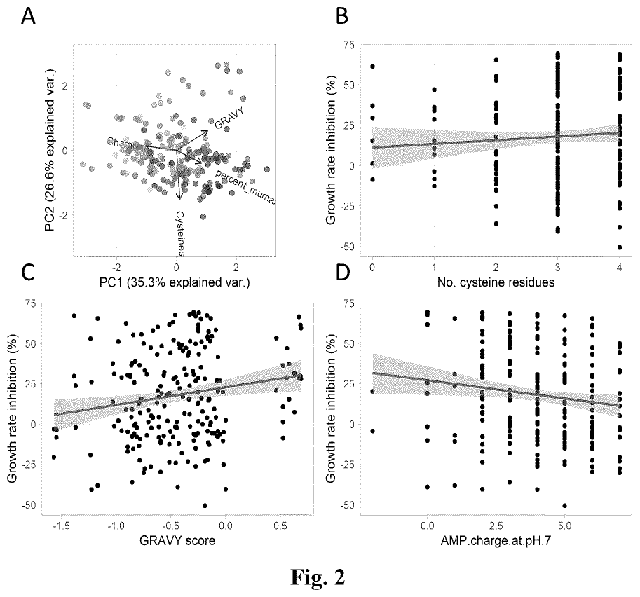

A high-throughput in vitro antimicrobial assay was performed to test the effect of 182 plant-derived active peptide sequences (Table 1) for growth inhibition using Liberibacter crescens strain BT-1, a cultivable surrogate of ‘Candidatus Liberibacter asiaticus’ (Leonard et al. 2012). While the individual plant-derived active peptide sequences had a wide range of effects on the growth of L. crescens strain BT-1 (FIG. 1A, purple distribution), the overall pattern trended towards inhibition compared to the positive control antimicrobial peptide polymyxin B (FIG. 1A, teal distribution). Note, the negative control in this case is growth of L. crescens strain BT-1 without any inhibitory molecule (FIG. 1A, green distribution). Overall, the plant-derived active peptide sequences were observed to inhibit growth rate of L. crescens strain BT-1 by up to 73% (FIG. 1B). An analysis of the physiochemical properties of the plant-derived active peptide sequences revealed weak but positive correlations between the L. crescens growth rate inhibition and number of cysteine residues, net peptide charge at pH 7 and calculated peptide solubility as a function of the GRAVY score (FIG. 2).

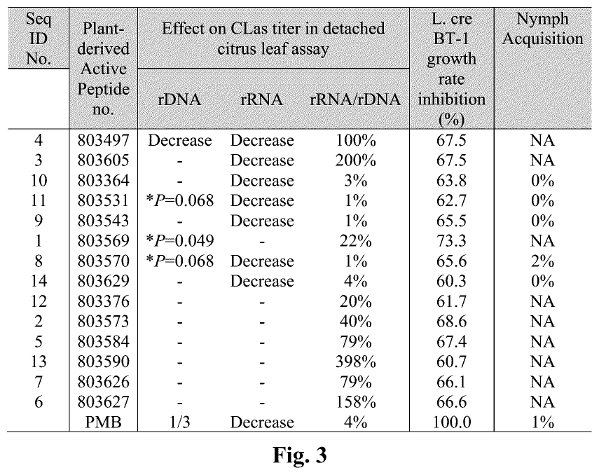

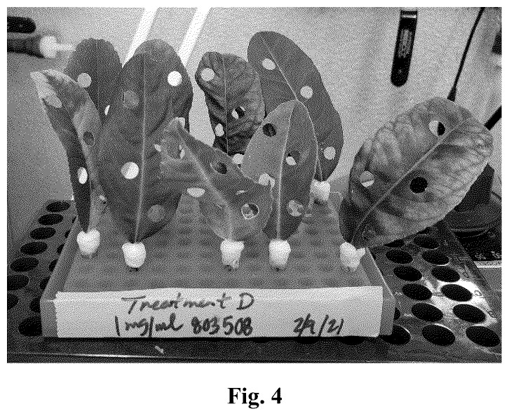

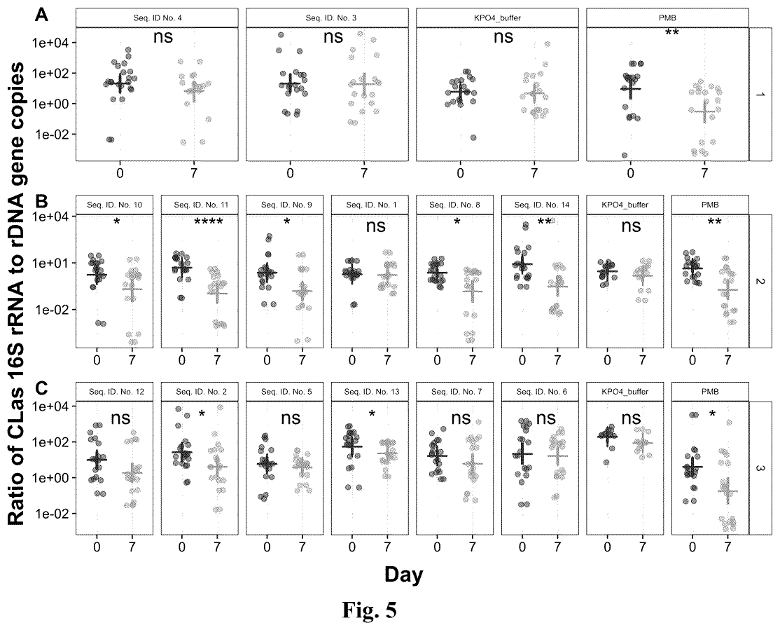

The 14 top-performing plant-derived active peptide sequences from the bacterial culture assays (FIG. 3) were selected for testing in an excised leaf assay (FIG. 4) to assess their effect on CLas that had already infected citrus trees. In these experiments, excised citrus leaves infected with CLas were removed from infected citrus trees. These leaves were carefully incubated for seven days in plant-derived active peptide-containing solutions containing plant-derived active peptides or potassium phosphate buffer solution as a control inside of an environmentally-controlled growth chamber. The antimicrobial peptide Polymyxin B (PMB) was in these assays as well. To our knowledge, PMB has not yet been reported in the literature to move in plant phloem or to inhibit CLas, but given its excellent performance as an antimicrobial peptide in the bacterial growth assays, that were included PMB as a control in all experiments. After seven days of treatment, the leaves were analyzed using RT-qPCR or qPCR to measure the total number of copies of CLas rRNA and rDNA they contained, respectively. CLas activity was estimated from the ratio CLas rRNA to CLas rDNA gene copies (CLas RNA:CLas DNA), where a higher value for the ratio indicates higher antibacterial activity for the plant-derived active peptides. The excised leaf assay revealed that 7/14 (50%) of the plant-derived active peptide sequences tested resulted in a significant reduction in the CLas 16S transcript-to-gene ratio over a seven-day incubation period, suggesting robust inhibition of CLas in planta by the plant-derived active peptides (FIG. 5).

Sequences of the top selected 14 plant-derived active peptides and other developed peptide sequences, GRAVY scores, #cysteine residues, Amp charge at ph7 are shown in the table below (Table 1).

TABLE 1

| | | | | | AMP | | | Percent |
|---|---|---|---|---|---|---|---|---|
| | | Uniprot/ | | | Score | | AMP | growth rate |
| SEQ. | | Genbank | | | (AMP | No. of | charge | inhibition |
| ID. | Sample | ID | 20 mer | GRAVY | predicted | Cys | at | L. |
| NO | ID | sequence | AMP | score | if >0.5) | residues | pH = 7 | crescens |
| 1 | 803569 | A7KH96 | CPRVSSH | -0.275 | 0.74 | 3 | 4 | 73.3 |
| | | | HIECVKG | | | | | |
| | | | FCTYWK | | | | | |
| 2 | 803573 | G7KYR3 | IGCDSIY | -0.515 | 0.736 | 3 | 0 | 68.6 |
| | | | YPISRPC | | | | | |
| | | | KTDKDC | | | | | |
| 3 | 803605 | A0A072ULH6 | CQQKFST | -0.29 | 0.66 | 3 | 0 | 67.5 |
| | | | QAEDLL | | | | | |
| | | | WCIRGYC | | | | | |
| 4 | 803497 | G7K5E0 | CPQIKSNI | -0.3 | 0.847 | 3 | 3 | 67.4 |
| | | | FRFKCIE | | | | | |
| | | | DRCKI | | | | | |
| 5 | 803584 | A0A072TYB9 | CAKGDG | -0.46 | 0.705 | 3 | 0 | 67.4 |
| | | | VCYKSCI | | | | | |
| | | | EEGFNRG | | | | | |
| 6 | 803627 | I3SA40 | LRFRSVF | 0.685 | 0.918 | 0 | 6 | 66.6 |
| | | | HIFASVL | | | | | |
| | | | AVAKKH | | | | | |
| 7 | 803626 | A0A072TFT6 | CVSKIICV | 0.69 | 0.929 | 4 | 4 | 66.2 |
| | | | LSQKPLC | | | | | |
| | | | RNHIC | | | | | |
| 8 | 803570 | A0A072VLD0 | YSSCATK | -1.17 | 0.739 | 4 | 1 | 65.5 |
| | | | EECKCPD | | | | | |
| | | | NKRPAC | | | | | |
| 9 | 803543 | G7JH11 | RGCKRD | -1.385 | 0.753 | 3 | 5 | 65.4 |
| | | | KDCPQFR | | | | | |
| | | | GVNIRCR | | | | | |
| 10 | 803364 | G7JX80 | VKCVLPR | -0.135 | 0.986 | 4 | 6 | 63.7 |
| | | | IARCIKY | | | | | |
| | | | RCQCRN | | | | | |
| 11 | 803531 | G7J0N2 | LYCNVGS | -0.125 | 0.763 | 4 | 4 | 62.7 |
| | | | HMECVK | | | | | |
| | | | HQCKCIK | | | | | |
| 12 | 803376 | A0A072UP37 | CPKFKKY | -0.62 | 0.959 | 3 | 6 | 61.6 |
| | | | NIRCRKG | | | | | |
| | | | FCVQVN | | | | | |
| 13 | 803590 | A0A072VAA4 | SFHPCKI | -0.25 | 0.694 | 3 | 3 | 60.7 |
| | | | NEHCTTY | | | | | |
| | | | KCLLTG | | | | | |
| 14 | 803629 | G7IT42 | QNLCVGS | 0.67 | 0.901 | 4 | 2 | 60.3 |
| | | | PLPLQCL | | | | | |
| | | | KFICRC | | | | | |

While some decline in CLas activity in potassium phosphate buffer was noted, only controls (0.1 mM, pH 5.8) over the seven-day incubation, antimicrobial peptide treatments tended to demonstrate greater declines in detectable CLas activity (e.g., see positive control PMB, FIG. 5).

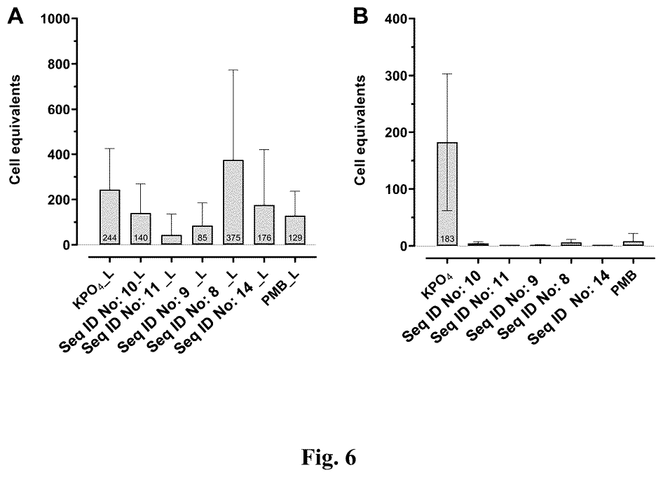

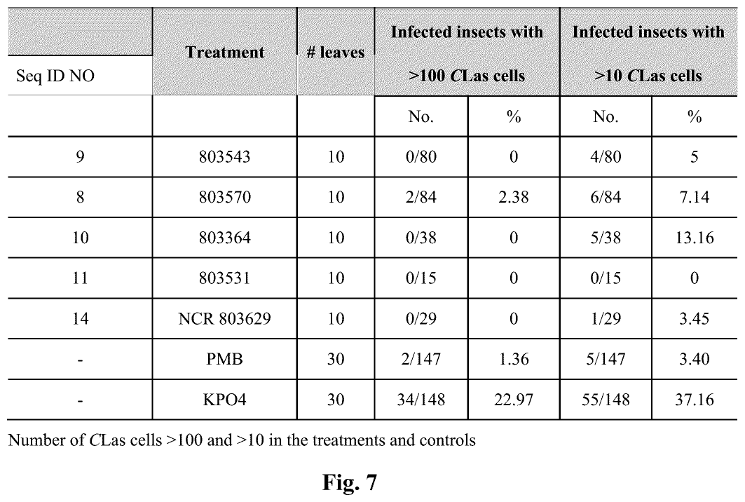

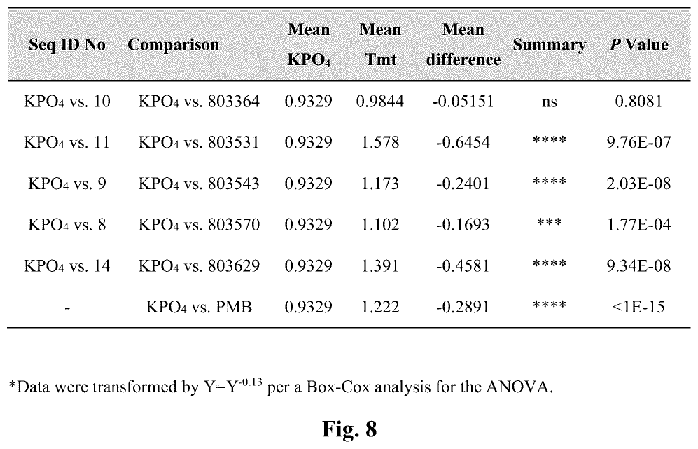

Plant-derived active peptides prevent the development of D. citri adults with high titers of CLas (FIG. 6, FIG. 7, FIG. 8). Acquisition of the CLas bacterium by psyllid nymphs is the first step required for tree-to-tree transmission to generate infected adult insects capable of transmitting CLas. CLas acquisition by healthy D. citri from infected leaves is quantifiable in laboratory assays (Igwe et al. Phytopathology (2021) 112:69-75). Due to the labor intensiveness of the excised leaf acquisition assay, the three plant-derived active peptide sequences with the lowest GRAVY scores (indicating most hydrophilic) were selected for this experiment: SEQ ID NO: 9 (803543), SEQ ID NO:8 (803570) and SEQ ID NO: 10 (803364) (Table 1). After the duration of the 21 day assay, a total of 80, 74, 84, 87 and 89 live adult psyllids were collected from the ten leaves exposed to peptides SEQ ID NO:9 (803543), SEQ ID NO: 10 (806634), SEQ ID NO: 8 (803570), PMB and the KPO4 treatment, respectively. Overall psyllids survived well on all treatments and controls, but low-levels of psyllid mortality was noted in some of the treatments. The three plant-derived active peptides and the PMB control reduced CLas titer in the excised leaves during the course of the experiment (FIG. 6A), although the differences were not statistically significant. However, the plant-derived active peptide sequence treatments blocked psyllid acquisition of CLas (FIG. 6B). In the buffer control treatment, 41.67% of psyllid that developed on leaves supplied with buffer developed CLas titers of more than 10 cells per insect (FIG. 7). A total of 28.33% of the insects developed high titers, more than 100 CLas cells per insect (FIG. 7). The plant-derived active peptides and PMB treatment prevented the development of high titer psyllid (FIG. 7), with peptides SEQ ID NO: 9 (803543) and SEQ ID NO: 10 (803364) completely blocking the development of psyllid with more than 100 CLas cells and peptide SEQ ID NO:8 (803570) resulting in only 2/84 adult insects with more than 100 CLas cell equivalents per insect (FIG. 7).

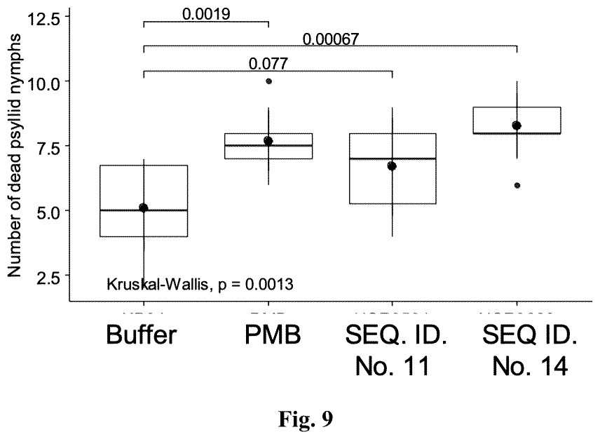

Nymph mortality observed with some treatments of plant-derived bioactive peptides (SEQ ID NO:11 and SEQ ID NO:14 are shown as an example (FIG. 9). Dead psyllids were counted daily during the assay and tallied up at the end of 21 days. Differences were quantified using a non-parametric Kruskal-Wallis ANOVA.

Described below are abbreviations used herein.

HLB=Huanglongbing

CLas=‘Candidatus Liberibacter asiaticus’

Ca.=Candidatus

CLam=‘Candidatus Liberibacter americanus’

CLaf=‘Candidatus Liberibacter africanus’

L. crescens=Liberibacter crescens

D. citri=Diaphorina citri

NCR=Nodule Cystine-Rich

BM7=Babaco Medium 7

BT-1=denotes type strain of the bacteria

UniProt=Universal Protein Resource

ANOVA=Analysis of Variance

Cq=quantification cycle

N-terminus=amino terminus, also amine terminus

C-terminus=carboxyl-terminus, also carboxy-terminus

—CONH2=amide

D-amino acid=dexter (right) enantiomer

ug=microgram

kg=kilogram

mg=milligram

SL=water-soluble liquids

EC=emulsifiable concentrates

EW=emulsions in water

SC=suspension concentrates

SE=suspoemulsion (?)

FS=Flowable concentrate for seed treatment

OD=optical density (?)

WG=water-dispersible granules

GR=granules

CS=capsule concentrates

FAO=Food and Agriculture Organization of the United Nations

WHO=World Health Organization

ISBN=International Standard Book Number

N-alkylpyrrolidones=the substituent is bonded to the nitrogen (amine)

N,N′-dimethylformamide=two methyl groups attached with a nitrogen

g=gram

h=hour

□C=degrees Celsius

DNA=Deoxyribonucleic Acid

qPCR=quantitative Polymerase Chain Reaction

M. truncatula=Medicago truncatula

AMP=antimicrobial peptide

CAMPR3=is a database of sequences, structures and family-specific signatures of prokaryotic and eukaryotic AMPs, which can be mined for discovery and design of AMPs. It is program/database for in silico aided computation of antimicrobial peptides by integrating composition-based features from known AMPs.

GRAVY=grand average of hydropathicity index

Biomatik=Peptide synthesis company

ACES=N-(2-Acetamido)-2-aminoethanesulfonic acid

FBS=fetal bovine serum

RT=room temperature

TMN-FH=type of buffer

rpm=revolutions per minute

ml=milliliters

v/v=volume per volume

nm=nanometers

O.D.=optical density

mg/ml=milligrams per milliliter

PMB=polymyxin B sulfate

pH=potential hydrogen

mM=millimolar

KPO4=potassium phosphate

(n=)=number of biological replicates

Fig.=Figure

° F.=degrees fahrenheit

μL=microliters

RNA=Ribonucleic acid

mm=millimeter

Hz=Hertz

MM=mixer mill model designation

RLT=Qiagen lysis buffer designation for RNA extraction

EtOH=ethanol

min=minute(s)

rDNA=ribosomal deoxyribonucleic acid

rRNA=ribosomal Ribonucleic acid

1×=1-fold concentrated

10×=10-fold concentrated

5×=five-fold concentrated

μM=micromolar

ng/μL=nanograms per microliter

ng=nanograms

hr=hour(s)

cDNA=complementary DNA

Inc=incorporated

g=gravity

CA=California

DNase=enzyme that degrades DNA

RNase=enzyme that degrades RNA

RT-minus=without reverse transcriptase added

RT-qPCR=Reverse transcription-quantitative polymerase chain reaction

SEQ ID NO=sequence identification number

Symbiont—a cell expressing a gene or genes of interest together with plant growth regulator genes sufficient to activate autonomous cell division, such as in an Agrobacterium T-DNA region

NA—Not applicable because it was not tested yet.

Thus, in view of the above, there is described (in part) the following:

An antibacterial pesticide composition comprising a peptide having an amino acid sequence selected from the group consisting of SEQ ID NO: 8, SEQ ID NO: 9. SEQ ID NO: 10, SEQ ID NO: 11, SEQ ID NO: 14 or mixtures thereof.

An antibacterial pesticide composition comprising a peptide having an amino acid sequence of SEQ ID NO: 8. An antibacterial pesticide composition comprising a peptide having an amino acid sequence of SEQ ID NO: 9. An antibacterial pesticide composition comprising a peptide having an amino acid sequence of SEQ ID NO: 10. An antibacterial pesticide composition comprising a peptide having an amino acid sequence of SEQ ID NO: 11. An antibacterial pesticide composition comprising a peptide having an amino acid sequence of SEQ ID NO: 14.

An antibacterial pesticide composition with a peptide having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12. SEQ ID NO: 13, SEQ ID NO: 14 or mixtures thereof.

An antibacterial pesticide having a peptide sequence of any of SEQ ID NO: 1 to SEQ ID NO: 14 described above with an acetylated residue. An antibacterial pesticide having a peptide sequence of any of SEQ ID NO: 1 to SEQ ID NO: 14 described above with an N-terminal acetylated peptide residue. An antibacterial pesticide having a peptide sequence of any of SEQ ID NO: 1 to SEQ ID NO: 14 described above with an amidated CONH2 C-terminus residue. An antibacterial pesticide described above having a peptide sequence of any of a peptide cyclization by a disulfide bridge of two cystine residues if any in the peptide. An antibacterial pesticide having a peptide sequence of any of SEQ ID NO: 1 to SEQ ID NO: 14 described above with a N-terminal modification, C-terminal modification, D-amino acid residue substitution, unnatural amino acid substitution, cyclization or backbone modification.

An antibacterial pesticide having a peptide sequence of any of SEQ ID NO: 1 to SEQ ID NO: 14 described above where the peptide or peptides are recombinantly expressed in a host cell, with a plant cell being one example host cell.

An antibacterial pesticide having a peptide sequence of any of SEQ ID NO: 1 to SEQ ID NO: 14 described above for treating citrus greening disease.

An antibacterial pesticide having a peptide sequence of any of SEQ ID NO: 1 to SEQ ID NO: 14 described above with a peptide having one or more amino acid residue substitutions to provide a sequence at least 85% identical to one of the sequences with SEQ ID NO: 1 to SEQ ID NO: 14.

An antibacterial pesticide composition comprising a peptide having an amino acid sequence selected from the group consisting of SEQ ID NO: 8, SEQ ID NO: 9. SEQ ID NO: 10, SEQ ID NO: 11, or SEQ ID NO: 14 with the peptide having one amino acid residue substitution in one of residues 1-20.

An antibacterial pesticide composition comprising a peptide having an amino acid sequence selected from the group consisting of SEQ ID NO: 8, SEQ ID NO: 9. SEQ ID NO: 10, SEQ ID NO: 11, or SEQ ID NO: 14 with the peptide having an amino acid residue substitution in two of residues 1-20.

An antibacterial pesticide composition comprising a peptide having an amino acid sequence selected from the group consisting of SEQ ID NO: 8, SEQ ID NO: 9. SEQ ID NO: 10, SEQ ID NO: 11, or SEQ ID NO: 14 with the peptide having an amino acid residue substitution in three of residues 1-20.

A method for treating citrus greening disease comprising treatment of a plant in need thereof with a composition comprising an effective amount of a peptide with SEQ ID NO: 8, SEQ ID NO: 9, SEQ ID NO: 10, SEQ ID NO: 11, SEQ ID NO: 14 or mixtures thereof.

A method for treating citrus greening disease by treatment of a plant in need thereof with a composition comprising an effective amount of a peptide with SEQ ID NO: 9.

A method for treating citrus greening disease by treatment of a plant in need thereof with a composition comprising an effective amount of a peptide with SEQ ID NO: 10.

A method for treating citrus greening disease by treatment of a plant in need thereof with a composition comprising an effective amount of a peptide with SEQ ID NO: 11.

A method for treating citrus greening disease by treatment of a plant in need thereof with a composition an effective amount of a peptide with SEQ ID NO: 14.

A method for treating citrus greening disease comprising treatment of a plant in need thereof with a composition comprising an effective amount of a peptide with SEQ ID NO: 8, SEQ ID NO: 9, SEQ ID NO: 10, SEQ ID NO: 11, SEQ ID NO: 14 or mixtures thereof with the peptide having an acetylated residue.

A method for treating citrus greening disease comprising treatment of a plant in need thereof with a composition comprising an effective amount of a peptide with SEQ ID NO: 8, SEQ ID NO: 9, SEQ ID NO: 10, SEQ ID NO: 11, SEQ ID NO: 14 or mixtures thereof with the peptide having an N-terminal acetylated peptide.

A method for treating citrus greening disease comprising treatment of a plant in need thereof with a composition comprising an effective amount of a peptide with SEQ ID NO: 8, SEQ ID NO: 9, SEQ ID NO: 10, SEQ ID NO: 11, SEQ ID NO: 14 or mixtures thereof with the peptide having an amidated CONH2 C-terminus residue.

A method for treating citrus greening disease comprising treatment of a plant in need thereof with a composition comprising an effective amount of a peptide with SEQ ID NO: 8, SEQ ID NO: 9, SEQ ID NO: 10, SEQ ID NO: 11, SEQ ID NO: 14 or mixtures thereof with the peptide having a cyclization by any disulfide bridge of two cystine residues present in the peptide.

A method for treating citrus greening disease comprising treatment of a plant in need thereof with a composition comprising an effective amount of a peptide with SEQ ID NO: 8, SEQ ID NO: 9, SEQ ID NO: 10, SEQ ID NO: 11, SEQ ID NO: 14 or mixtures thereof with the peptide having a N-terminal modification, C-terminal modification, D-amino acid substitution, unnatural amino acid substitution, cyclization, backbone modification or nanoparticle formulation.

A prophylactic method of treatment of citrus greening disease by treatment of a plant in need thereof with a composition comprising an effective amount of a peptide with SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12. SEQ ID NO: 13, or SEQ ID NO: 14.

A method for killing a psyllid vector of citrus greening disease by treatment of a plant in need thereof with a composition comprising an effective amount of a peptide with SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12. SEQ ID NO: 13 or SEQ ID NO: 14. The method for killing a psyllid vector described above when the psyllid is Diaphorina citri.

A method for treating citrus greening disease by treating a plant in need thereof with a symbiont forming inoculum comprising at least one polynucleotide encoding one or more phytohormone biosynthetic enzymes and a polynucleotide expressing a peptide effective in treating citrus greening disease, wherein the phytohormone biosynthetic enzyme is at least one cytokinin biosynthetic enzyme and/or an auxin biosynthetic enzyme.

The method for treating citrus greening disease by treating a plant in need thereof with a symbiont forming inoculum described above wherein the peptide effective in treating citrus greening disease has a sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12. SEQ ID NO: 13 or SEQ ID NO: 14 or mixtures thereof.

The method for treating citrus greening disease by treating a plant in need thereof with a symbiont forming inoculum described above wherein the polynucleotides encoding a phytohormone biosynthetic enzyme and the polynucleotide of interest are comprised in a plant cell or a bacterial cell.

The method for treating citrus greening disease by treating a plant in need thereof with a symbiont forming inoculum described above wherein the phytohormone biosynthetic enzyme is an indole-3-acetamide hydrolase (iaaH) (E.C. Number: EC 3.5.1.4), amidase 1 (EC 3.5.1.4), a tryptophan 2-monooxygenase (IaaM) (EC 1.13.12.3), an indole-3-lactate synthase (EC 1.1.1.110), a L-tryptophan-pyruvate aminotransferase 1 (EC 2.6.1.99), a tryptophan aminotransferase-related protein 1 (EC 2.6.1.27), indole-3-acetaldehyde oxidase (EC 1.2.3.7), a tryptophan decarboxylase 1/tryptophan decarboxylase 2 (EC4.1.1.105), an isopentenyl transferase (Ipt) and/or a Tzs (EC 2.5.1.27).

The method for treating citrus greening disease by treating a plant in need thereof with a symbiont forming inoculum described above wherein the polynucleotides encoding a phytohormone biosynthetic enzyme and the polynucleotide expressing a peptide effective in treating citrus greening disease are comprised in a single nucleic acid construct or in two or more nucleic acid constructs.

The method for treating citrus greening disease by treating a plant in need thereof with a symbiont forming inoculum described above wherein the one or more nucleic acid constructs are comprised in one or more vectors selected from a group consisting of a plasmid, a T-DNA, a bacterial artificial chromosome, viral vector, or a binary-bacterial artificial chromosome.

A symbiont forming inoculum to treat citrus greening disease comprising a polynucleotide for expression of a peptide of sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12. SEQ ID NO: 13 or SEQ ID NO: 14, and a polynucleotide encoding one or more phytohormone biosynthetic enzymes; wherein the phytohormone biosynthetic enzyme is at least one cytokinin biosynthetic enzyme and/or an auxin biosynthetic enzyme.

A transgenic citrus plant transformed with a recombinant construct comprising a nucleic acid that encodes a polypeptide having at least 90% identity to the sequence SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12. SEQ ID NO: 13 or SEQ ID NO: 14.

A citrus plant with a recombinant construct comprising a nucleic acid that encodes a polypeptide with peptide sequences listed above wherein expression of the polypeptide confers an altered trait in the plant of resistance to citrus greening disease.

A citrus plant with a recombinant construct comprising a nucleic acid that encodes a polypeptide with peptide sequences listed above wherein expression of the polypeptide confers an altered trait in the plant treatment of citrus greening disease.

A citrus plant with a recombinant construct comprising a nucleic acid that encodes a polypeptide with peptide sequences listed above wherein expression of the polypeptide confers an altered trait in the plant of decreased CLas transmission.

Sequences of the 182 plant-based active peptides synthesized and evaluated in the in vitro antimicrobial assays (Table 2 below).

TABLE 2

| SEQ | Number | | Origination |
|---|---|---|---|
| ID | (and SEQ | | |
| NO: | ID NO:) | 20 mer Sequence | Protein Name |
| 121 | 803362 | IPRCIKYKCLCGNGVGKRWS | AOA072VBA6 |
| 122 | 803363 | VYRCVGNYCRAVKIRRWNLG | AOA072VJ77 |
| 10 | 803364 | VKCVLPRIARCIKYRCQCRN | G7JX80 |
| | (SEQ ID | | |
| | NO: 10) | | |
| 178 | 803365 | CTNKIKCVPPRIAQCFRFKC | AOA072VHP7 |
| 145 | 803366 | CPKLYGANFRCRKGTCVPPI | AFK48426 |
| 68 | 803367 | CVHKRCQLPQIPKCVGKKCR | G713P8 |
| 89 | 803368 | CYKKYPFIPWGKVRCVKGRC | A7KHD6 |
| 82 | 803369 | CPKPPRINIRINIRCRKGFC | G7JR68 |
| 139 | 803370 | KICHPPQIRKCVSKICKCRL | G7KIM9 |
| 49 | 803371 | CPRKNRHVVKCRKGYCVGVQ | AOA072UTU6 |
| 44 | 803372 | CPSWKNYTGRCRKGFCILNR | AOA072TVR4 |
| 62 | 803374 | FKPKCRFRSCTCSNLKVWKG | AOA072U6G3 |
| 90 | 803375 | KINNVRCRKGFCIQIHKFTP | G7JM28 |
| 12 | 803376 | CPKFKKYNIRCRKGFCVQVN | AOA072UP37 |
| | (SEQ ID | | |
| | NO: 12) | | |
| 140 | 803377 | CVTKVKCGLPRTPKCRNYIC | AOA072TR97 |
| 182 | 803379 | VGCRLQREKPRCVNLVCRCL | G714L5 |
| 55 | 803390 | CKHVRDCPKGIWRSCRYKCI | G7JOL4 |
| 167 | 803391 | CQATTKCVLPRVPRCIKYKC | G7KIL8 |
| 70 | 803393 | LVTKCFKKHCRCRKPGLQVQ | G71WA4 |
| 158 | 803395 | RCVRRMCKCLPIGWRKYFVP | A7KHG6 |
| 98 | 803398 | CKQVKGYIARCRKGYCMQSV | A2Q2R4 |
| 164 | 803401 | CVARINCVLPRKPQCRNYAC | AOA072TFE5 |
| 48 | 731163 | KDCPKLHKVNVRCRKGKCVA | AOA072UHC2 |
| 85 | 803405 | CPRFKNNNVRCRKGFCVNLC | AOA072U9N1 |
| 96 | 803406 | KDCPWAKNYVLKCRKGYCVF | AOA072TXJ8 |
| 92 | 803407 | CKDKFPGNKYPIKCINGIFY | AOA072U8H9 |
| 105 | 803408 | RKCLRSTCVCRKFRFTGFYY | G71X58 |
| 162 | 803409 | LRANCVDRGVCKCVPVWWRK | AOA072UT45 |
| 118 | 803411 | LKNEIFHWKKQICNIHFAKL | 13SB50 |
| 56 | 803413 | CPKVQHGYKLRCRKGQCVHI | AOA072TV89 |
| 109 | 803416 | CRAGSHRVQCIKHQCKCVRI | G7JOM9 |
| 61 | 803420 | CPKHRGVNGKCRKGYCVGVG | AOA072UHA9 |
| 181 | 803423 | GENFCISRFRVKCWRFKCFC | G71VN8 |
| 80 | 803426 | ITPSPKFKWKCINKRCLYIR | G7JY89 |
| 148 | 803427 | AFYGSMRCVKGFCKHLKDVK | AOA072TW61 |
| 161 | 803428 | LLKTYVWKCVKNECYFFAKK | A7KHE6 |
| 176 | 803429 | FIKCVCGVYGPIRERRLYQS | A7KHD9 |
| 40 | 803430 | CKQHRGFNFRCRSGNCIPIR | AOA072TFL3 |
| 28 | 803433 | KDCPSVKNYIGRCRKGYCQA | 13SIU5 |
| 126 | 803435 | ISCKDHFECRRKINILRCIY | A7KHE7 |
| 110 | 803436 | CPKSVLRVWRCINNYCRPVR | AOA072UQA7 |
| 150 | 803438 | LKKYIYSPGARRVASSCGVP | AOA072TVT5 |
| 137 | 803441 | VKKPLKMWCIRQTCFYGFGK | AOA072UBS9 |
| 45 | 803450 | CQNHRGFNFRCRKGNCVAKI | AOA072UWBO |
| 183 | 803454 | KTLCHSPGKAKYFCSLLSLK | AOA072UJ01 |
| 124 | 803456 | KDCRSFLCYSPKFPVCKRGI | QIRU35 |
| 66 | 803457 | CSKVEYGYKLRCRKGRCVHI | AOA072V149 |
| 34 | 803459 | CPKVSQYNIRCRKGQCARIR | AOA072U3K5 |
| 185 | 803460 | LVQRYRCINGKCNLSFVSYG | A7KHD7 |
| 60 | 803461 | CPRKKKFSVTCRKGFCAEIR | AOA072VU91 |
| 166 | 803464 | KFPGPSKYPIKCMKGICKCV | AOA072U873 |
| 112 | 803465 | VNNRCYLHGKPSCLNGQCAC | AOA072TWJ1 |
| 72 | 803466 | CAPEKYYNIRCRKGFCVQIR | AOA072TYL4 |
| 112 | 803467 | CVTKIKCVLPRKPECRNNAC | A7KHA5 |
| 132 | 803468 | CPNPKYGKCLDNKCICQLIW | AOA072VAL4 |
| 87 | 803469 | CKSDKDCKDIIIYRYILKCR | A7KH67 |
| 114 | 803470 | WTKIYKCIDNKCRYSVVKGL | G7JOLO |
| 174 | 803471 | CIGYWCPLSIQPRSTKPICR | AOA072U9R9 |
| 31 | 803472 | KDCPKLGRANIRCREGYCVR | AOA072TDLO |
| 94 | 803473 | DCPKFGRVNVRCRKGNCVPI | A7KHF4 |
| 127 | 803474 | CVTRIKCVLPRKPECRNYAC | A7KHF9 |
| 142 | 803476 | IPYCRFREKNVRFGSPLGLC | AOA072VK07 |
| 32 | 803477 | NYVHKCINNRCEWIKIIRRR | G7K767 |
| 116 | 803478 | CFIDGNCPRNMCKVRWKLRL | G71ZE7 |
| 106 | 803479 | VHCQKYKCSPGLYPTCINGW | AOA072TD23 |
| 172 | 803480 | IFLCSFIAAKNIDGRNNPTR | G71W93 |
| 99 | 803481 | CTWNLCRQPWVQKCRLHMCS | A7KHC9 |
| 152 | 803482 | WFKIYRCEKGICRYHKLWIV | G715S5 |
| 93 | 803483 | KIKCVLPRTPQCRNEACGCY | A7KHA9 |
| 115 | 803484 | DCPKPLRFNIRCRKGFCVRI | AOA072UGSO |
| 141 | 803485 | FVRCKMNRCIYSRVQPPWAC | AOA072V6Y7 |
| 111 | 803486 | CPKSEFRKWVCINNICRKMC | AOA072UL49 |
| 179 | 803487 | CEKLYPGNKKPLICNIGYCL | AOA072UKJ5 |
| 102 | 803488 | CTRRKGFSVTCRKGFCVEFK | AOA072VJQ8 |
| 83 | 803489 | CAYPHVLRCIGKNCAENKNG | G7KV48 |
| 146 | 803490 | RVAWCVNNKCECVLTYGPKY | G7K8T1 |
| 133 | 803494 | CKHPFKPRCLTHSCVCRLWG | AOA072UWG7 |
| 35 | 803496 | RVKRFKCVKGECRWTRMSYA | A7KH82 |
| 4 | 803497 | CPQIKSNIFRFKCIEDRCKI | G7K5EO |
| | (SEQ ID | | |
| | NO: 4) | | |
| 135 | 803498 | ADCLGEKCLPPKRYWCRIIT | AOA072VH74 |
| 24 | 803499 | DCPKYQRANIRCRKGQCVRI | AOA072TDG4 |
| 36 | 803500 | DCPQFRRANIRCRKGQCVKL | AOA072UJ73 |
| 101 | 803501 | IRCRQGLKELASDGLRVTGY | G7KWZ1 |
| 149 | 803502 | AQCIYPACFKDHMCRQLKCS | AOA072TNG5 |
| 39 | 803503 | GRTYKCINNKCRYPKLLKPI | AOA072V4B1 |
| 156 | 803504 | CRRNIDCPSYLCVAPKVPRC | A7KHF7 |
| 27 | 803505 | GRKCKQNSDCSKEICVFPWK | AOA072V8J4 |
| 143 | 803506 | CRTPLKPKCMYRTFCKCKVV | AOA072VH12 |
| 128 | 803507 | ADCPISKLNMYNWRCIKSSC | A7KH69 |
| 144 | 803508 | CRPPLKSKCMYKTNCKCIAV | AOA072TQJO |
| 157 | 803509 | GRMVCGGRSRFGFRSCGGYL | G7JQH7 |
| 59 | 803510 | CPPRTRLILYKCRNRKCVSY | AOA072V4D2 |
| 134 | 803511 | LANKTFYLKCIDKKCEWTVT | G7KEA3 |
| 147 | 803512 | CPKSQLEMFAWKCVKNGCHL | G7KAD9 |
| 53 | 803513 | SFYKCIDNLCKRFRRQKHLV | G7KJJ5 |
| 160 | 803514 | GNNLCEGRWIPKCLKPYFLF | AOA072V8P6 |
| 108 | 803515 | CKPPFNPRCHNHICICRLWG | G8A361 |
| 175 | 803516 | CVYPYAVQCIHRYCKCLKSR | A7KH93 |
| 170 | 803517 | GVFVSCNSHIHCRVNNHKIG | A7KH74 |
| 46 | 803518 | CLALDRKRPIGKKFPCKADK | AOA072UTUO |
| 168 | 803519 | LKLKCDRHTILTCFWRHCYC | AOA072V6P6 |
| 42 | 803520 | LWDKNYAHRCVNNICEWVKK | A7KHF2 |
| 177 | 803521 | VISIHCRTNADCPRNMCKIG | G71Z19 |
| 188 | 803522 | LPYCDRNRFCTSRGYIVCIT | AOA072UT81 |
| 100 | 803523 | CEKIYPGNKKPLICSTGYCY | A7KHB8 |
| 23 | 803524 | CRRKYRGANKHLLWCNDGYC | G7JPA1 |
| 20 | 803525 | CPDDKKIKGRCRKGFCTNGW | AOA072UUK9 |
| 154 | 803526 | CPPSYTKIYRCIDNKCRLVL | G7JOL3 |
| 25 | 803529 | KDCPKKMGTVGKCRKGYCAQ | AOA072TY77 |
| 11 | 803531 | LYCNVGSHMECVKHQCKCIK | G7JON2 |
| | (SEQ ID | | |
| | NO: 11) | | |
| 120 | 803534 | IHYYCPPSKVPYCQVDRCGC | AOA072U164 |
| 19 | 803536 | CPNGRNYIGRCRKGHCQQRL | G7KYR1 |
| 41 | 803537 | DCPNMKHYKAKCRKGFCISS | A7KHA4 |
| 29 | 803540 | WAYIYVCEKNKCRYHFKSGR | AOA072VCR5 |
| 9 | 803543 | RGCKRDKDCPQFRGVNIRCR | G7JH11 |
| | (SEQ ID | | |
| | NO: 9) | | |
| 95 | 803546 | RYCVYPTIPLCDVKHCRCRR | G7JP15 |
| 15 | 803550 | DCKPKRGVNFRCRKGKCYPR | AOA072UIN6 |
| 73 | 803566 | CRKKFAGANQHLLWCNNGYC | G7JPA2 |
| 76 | 803567 | KCMYKSICKCIREFSKRDYV | AOA072U8J5 |
| 88 | 803568 | CVTKIKCVLPRKPECRNTQC | 13SFJ9 |
| 1 | 803569 | CPRVSSHHIECVKGFCTYWK | A7KH96 |
| | (SEQ ID | | |
| | NO: 1) | | |
| 8 | 803570 | YSSCATKEECKCPDNKRPAC | AOA072VLDO |
| | (SEQ ID | | |
| | NO: 8) | | |
| 38 | 803571 | LWDRNYAHRCVNNICEWVKK | A7KHF1 |
| 54 | 803572 | GCIVDPRCPYQQCRRPLYCR | KEH17417.1 |
| 2 | 803573 | IGCDSIYYPISRPCKTDKDC | G7KYR3 |
| | (SEQ ID | | |
| | NO:) | | |
| 123 | 803574 | CRDYLCARPTVGKCIYDYCH | AOA072TVW8 |
| 136 | 803575 | CRKHMCTPYGQLVRCINSTC | AOA072VB20 |
| 37 | 803576 | DCPKLHRSNVRCRKGQCVQI | A7KH71 |
| 119 | 803577 | RPQCVINTCRCRPLRFSGFY | G71X60 |
| 64 | 803578 | CKETGYTKGGHCRDEGVVCC | AOA072UOTO |
| 69 | 803579 | CQKKYPGPYEHLLKCVSGYC | AOA072UJP9 |
| 103 | 803580 | CQRYRHKLATRMICNQGFCL | G7JLZ7 |
| 65 | 803581 | CQKKFPGSNQHLLWCNNGFC | AOA072U183 |
| 79 | 803582 | DCPAPPRFNIRCRKGYCVRI | AOA072UWB4 |
| 51 | 803583 | CGSNSDCLWEKCLPPNKHWC | AOA072VGJ2 |
| 5 | 803584 | CAKGDGVCYKSCIEEGFNRG | AOA072TYB9 |
| | (SEQ ID | | |
| | NO: 5) | | |
| 33 | 803585 | SFPCKTNSDCPSYLCHYPKN | AOA072VK32 |
| 138 | 803586 | IDKEYTVCSLHSDCKAYVCQ | AOA072TJN6 |
| 81 | 803587 | ILCKVHEDCPQKSTHKYYCI | AOA072UYE1 |
| 91 | 803588 | CDSAYLPLSRSCITDKDCSR | AOA072U6W1 |
| 77 | 803589 | CYKRYPRWSLLPNYCIEGSC | AOA072TQEO |
| 13 | 803590 | SFHPCKINEHCTTYKCLLTG | AOA072VAA4 |
| | (SEQ ID | | |
| | NO: 13) | | |
| 97 | 803591 | CKTKVDCPQHKKYIAECIFG | AOA072U2G4 |
| 43 | 803592 | PPHNIKCRKGHCVPIGKPFK | AOA072UOD8 |
| 22 | 803593 | DCRRRGSNQYWVYKCINHGC | G713W5 |
| 30 | 803594 | RRGTNKYFIHKCIDYRCQWI | G714S7 |
| 173 | 803596 | IGCKTSEDCPYLGKCIEDFC | AOA072TIJO |
| 125 | 803597 | VKIHFYCPPPKVPYCRVDRC | AOA072UK35 |
| 57 | 803598 | CSSAYTPRCRHRTCVCLNND | G7J277 |
| 171 | 803603 | CKNPDDVPRCIFPVCHCIKS | AOA072UWC9 |
| 104 | 803604 | CPKVEKPITMKCINNYCKYF | A7KH81 |
| 3 | 803605 | CQQKFSTQAEDLLWCIRGYC | AOA072ULH6 |
| | (SEQ ID | | |
| | NO: 3) | | |
| 107 | 803606 | KQMCHLNQTPKCLKNICKCV | A7KHB2 |
| 17 | 803607 | NNFCRYREAVRRLRPPLRKK | G713U8 |
| 74 | 803608 | CYIQYPKTPFGHMECYKGSC | AOA072V478 |
| 21 | 803609 | CKIDKDCPRNPPLNIRCRKS | AOA072VEW9 |
| 26 | 803610 | RPQFRKSNVRCRNGYCVNLG | G7KWY8 |
| 47 | 803611 | CPQKSTHKYYCVDDKCFLYY | G7J9F1 |
| 67 | 803612 | FLAESNWSQRNDITKIHCIK | AOA072TGW4 |
| 71 | 803613 | CPKVEKPLYMYCGNHWCAYK | G7KNA5 |
| 86 | 803614 | IKCKVDEDCPNVFTYSWKC | AOA072UJR7 |
| 75 | 803615 | ANRGLQCLNGECKSSRIIKS | AOA072TPC7 |
| 129 | 803616 | CPKYRDLLYVFKCIDKRCEL | AOA072UFG6 |
| 311 | 803617 | INGVKSLLLIKVRSFIPCQR | A7KHBO |
| 327 | 803618 | IATGARRKNFFFIILKFSSL | AOA072VHX2 |
| 304 | 803619 | VRLPLVPISVGKSIQRFARF | AOA072VUP8 |
| 331 | 731156 | FLFAMNVAGFGWKCIKRRCV | AOA072UE40 |
| 324 | 803620 | IAAKNVVKNPGPCFVMGACS | AOA072UMU2 |
| 297 | 731160 | VCLNYKFPTCVGKKCYCLSA | AOA072VAZ7 |
| 336 | 731159 | LPALQRAVMKCIQGFCKIHI | AOA072UIS4 |
| 308 | 803621 | FMCPSYLAVKCIGRLCRCGR | G71W92 |
| 317 | 731162 | LIKIVHLWKKIRVRVDVVKA | AOA072UIG4 |
| 305 | 803622 | FISLFFIAKNVEGRVKCIKD | AOA072V8QO |
| 320 | 803623 | CTGPQIPKCVSHICFCLSSG | A7KH94 |
| 316 | 731134 | GLRRASTITALPINVFAIYK | AOA072US80 |
| 312 | 803624 | FLSLFLVATNIEGKFQKCCK | AOA072VGT6 |
| 315 | 803625 | GLAPKCFVSFALARFLSEGR | AOA072TQP5 |
| 7 | 803626 | CVSKIICVLSQKPLCRNHIC | AOA072TFT6 |
| | (SEQ ID | | |
| | NO: 7) | | |
| 6 | 803627 | LRFRSVFHIFASVLAVAKKH | 13SA40 |
| | (SEQ ID | | |
| | NO: 6) | | |
| 306 | 803628 | FLSITVYGYIPGIVNKPCKT | 13SVG9 |
| 14 | 803629 | QNLCVGSPLPLQCLKFICRC | G71T42 |
| | (SEQ ID | | |
| | NO: 14) | | |
| 328 | 803631 | VRCIKETCKCIKILEPINVV | G7JAL3 |

Sequences of the 623 calculated antimicrobial peptides, their GRAVY score to measure hydrophobicity, their antimicrobial peptide score to calculate rank the quality of the peptide as an anti-microbial, the number of cysteine resides, and the charge of the 20-mer peptide at pH 7 (Table 3).

TABLE 3

| | | | | AMP | | |
|---|---|---|---|---|---|---|
| | | | | Score | | AMP |
| SEQ | | | | (AMP | No. | charge |
| ID | Origination | | GRAVY | predicted | Cys | at |
| NO: | Protein | 20 mer Sequence | score | if >0.5) | Residues | pH = 7 |
| 15 | A0A072UIN6 | DCKPKRGVNFRCRKGKCYPR | -1.57 | 0.748 | 3 | 7 |
| 16 | A0A072V1J1 | RKGYTAKRLYEYETQDITQT | -1.565 | NA | 0 | 1 |
| 17 | G7I3U8 | NNFCRYREAVRRLRPPLRKK | -1.545 | 0.655 | 1 | 7 |
| 18 | G7J9F3 | CKTDKECPNTSTHKYKCIND | -1.505 | 0.675 | 3 | 2 |
| 19 | G7KYR1 | CPNGRNYIGRCRKGHCQQRL | -1.37 | 0.759 | 3 | 6 |
| 20 | A0A072UUK9 | CPDDKKIKGRCRKGFCTNGW | -1.235 | 0.773 | 3 | 4 |
| 21 | A0A072VEW9 | CKIDKDCPRNPPLNIRCRKS | -1.225 | 0.653 | 3 | 4 |
| 22 | G7I3W5 | DCRRRGSNQYWVYKCINHGC | -1.175 | 0.68 | 3 | 4 |
| 23 | G7JPA1 | CRRKYRGANKHLLWCNDGYC | -1.12 | 0.774 | 3 | 5 |
| 24 | A0A072TDG4 | DCPKYQRANIRCRKGQCVRI | -1.03 | 0.839 | 3 | 5 |
| 25 | A0A072TY77 | KDCPKKMGTVGKCRKGYCAQ | -1.02 | 0.768 | 3 | 5 |
| 26 | G7KWY8 | RPQFRKSNVRCRNGYCVNLG | -1.02 | 0.651 | 2 | 5 |
| 27 | A0A072V8J4 | GRKCKQNSDCSKEICVFPWK | -0.98 | 0.819 | 3 | 3 |
| 28 | I3S1U5 | KDCPSVKNYIGRCRKGYCQA | -0.95 | 0.915 | 3 | 4 |
| 29 | A0A072VCR5 | WAYIYVCEKNKCRYHFKSGR | -0.93 | 0.755 | 2 | 5 |
| 30 | G7I4S7 | RRGTNKYFIHKCIDYRCQWI | -0.915 | 0.678 | 2 | 5 |
| 31 | A0A072TDL0 | KDCPKLGRANIRCREGYCVR | -0.91 | 0.885 | 3 | 4 |
| 32 | G7K767 | NYVHKCINNRCEWIKIIRRR | -0.9 | 0.88 | 2 | 6 |
| 33 | A0A072VK32 | SFPCKTNSDCPSYLCHYPKN | -0.895 | 0.703 | 3 | 2 |
| 34 | A0A072U3K5 | CPKVSQYNIRCRKGQCARIR | -0.895 | 0.903 | 3 | 6 |
| 35 | A7KH82 | RVKRFKCVKGECRWTRMSYA | -0.87 | 0.85 | 2 | 6 |
| 36 | A0A072UJ73 | DCPQFRRANIRCRKGQCVKL | -0.86 | 0.838 | 3 | 5 |
| 37 | A7KH71 | DCPKLHRSNVRCRKGQCVQI | -0.855 | 0.73 | 3 | 5 |
| 38 | A7KHF1 | LWDRNYAHRCVNNICEWVKK | -0.855 | 0.739 | 2 | 3 |
| 39 | A0A072V4B1 | GRTYKCINNKCRYPKLLKPI | -0.845 | 0.829 | 2 | 6 |
| 40 | A0A072TFL3 | CKQHRGFNFRCRSGNCIPIR | -0.835 | 0.916 | 3 | 6 |
| 41 | A7KHA4 | DCPNMKHYKAKCRKGFCISS | -0.835 | 0.757 | 3 | 5 |
| 42 | A7KHF2 | LWDKNYAHRCVNNICEWVKK | -0.825 | 0.78 | 2 | 3 |
| 43 | A0A072U0D8 | PPHNIKCRKGHCVPIGKPFK | -0.81 | 0.688 | 2 | 7 |
| 44 | A0A072TVR4 | CPSWKNYTGRCRKGFCILNR | -0.79 | 0.969 | 3 | 5 |
| 45 | A0A072UWB0 | CQNHRGFNFRCRKGNCVAKI | -0.785 | 0.91 | 3 | 6 |
| 46 | A0A072UTU0 | CLALDRKRPIGKKFPCKADK | -0.78 | 0.785 | 2 | 5 |
| 47 | G7J9F1 | CPQKSTHKYYCVDDKCFLYY | -0.77 | 0.647 | 3 | 2 |
| 48 | A0A072UHC2 | KDCPKLHKVNVRCRKGKCVA | -0.75 | 0.939 | 3 | 7 |
| 49 | A0A072UTU6 | CPRKNRHVVKCRKGYCVGVQ | -0.74 | 0.972 | 3 | 7 |
| 50 | G7KE56 | WQKFHTYKCINQKCKWVLRF | -0.725 | 0.964 | 2 | 6 |
| 51 | A0A072VGJ2 | CGSNSDCLWEKCLPPNKHWC | -0.72 | 0.706 | 4 | 1 |
| 52 | A0A072UST5 | KDCPKLRGGSFRCRKGKCVL | -0.705 | 0.951 | 3 | 6 |
| 53 | G7KJJ5 | SFYKCIDNLCKRFRRQKHLV | -0.705 | 0.803 | 2 | 6 |
| 54 | KEH17417.1 | GCIVDPRCPYQQCRRPLYCR | -0.69 | 0.738 | 4 | 3 |
| 55 | G7J0L4 | CKHVRDCPKGIWRSCRYKCI | -0.685 | 0.949 | 4 | 6 |
| 56 | A0A072TV89 | CPKVQHGYKLRCRKGQCVHI | -0.68 | 0.928 | 3 | 7 |
| 57 | G7J277 | CSSAYTPRCRHRTCVCLNND | -0.665 | 0.669 | 4 | 3 |
| 58 | A0A072UAE3 | PCATSDDCLKNMCRPPLTPR | -0.66 | 0.514 | 3 | 1 |
| 59 | A0A072V4D2 | CPPRTRLILYKCRNRKCVSY | -0.64 | 0.809 | 3 | 6 |
| 60 | A0A072VU91 | CPRKKKFSVTCRKGFCAEIR | -0.625 | 0.901 | 3 | 6 |
| 61 | A0A072UHA9 | CPKHRGVNGKCRKGYCVGVG | -0.61 | 0.923 | 3 | 6 |
| 62 | A0A072U6G3 | FKPKCRFRSCTCSNLKVWKG | -0.61 | 0.966 | 3 | 6 |
| 63 | A7KHG5 | DCPKLRRANVRCRKSYCVPI | -0.605 | 0.904 | 3 | 5 |
| 64 | A0A072U0T0 | CKETGYTKGGHCRDEGVVCC | -0.595 | 0.722 | 4 | 1 |
| 65 | A0A072UI83 | CQKKFPGSNQHLLWCNNGFC | -0.595 | 0.711 | 3 | 3 |
| 66 | A0A072VI49 | CSKVEYGYKLRCRKGRCVHI | -0.595 | 0.904 | 3 | 6 |
| 67 | A0A072TGW4 | FLAESNWSQRNDITKIHCIK | -0.59 | 0.637 | 1 | 2 |
| 68 | G7I3P8 | CVHKRCQLPQIPKCVGKKCR | -0.585 | 0.975 | 4 | 7 |
| 69 | A0A072UJP9 | CQKKYPGPYEHLLKCVSGYC | -0.565 | 0.719 | 3 | 3 |
| 70 | G7IWA4 | LVTKCFKKHCRCRKPGLQVQ | -0.56 | 0.944 | 3 | 7 |
| 71 | G7KNA5 | CPKVEKPLYMYCGNHWCAYK | -0.555 | 0.635 | 3 | 3 |
| 72 | A0A072TYL4 | CAPEKYYNIRCRKGFCVQIR | -0.555 | 0.895 | 3 | 4 |
| 73 | G7JPA2 | CRKKFAGANQHLLWCNNGYC | -0.55 | 0.748 | 3 | 4 |
| 74 | A0A072V478 | CYIQYPKTPFGHMECYKGSC | -0.535 | 0.655 | 3 | 2 |
| 75 | A0A072TPC7 | ANRGLQCLNGECKSSRIIKS | -0.53 | 0.628 | 2 | 3 |
| 76 | A0A072U8J5 | KCMYKSICKCIREFSKRDYV | -0.52 | 0.743 | 3 | 4 |
| 77 | A0A072TQE0 | CYKRYPRWSLLPNYCIEGSC | -0.515 | 0.694 | 3 | 2 |
| 78 | A0A072UHP8 | CPKFYGSNVRCRKGKCVQLG | -0.505 | 0.979 | 3 | 5 |
| 79 | A0A072UWB4 | DCPAPPRFNIRCRKGYCVRI | -0.505 | 0.71 | 3 | 4 |
| 80 | G7JY89 | ITPSPKFKWKCINKRCLYIR | -0.495 | 0.921 | 2 | 6 |
| 81 | A0A072UYE1 | ILCKVHEDCPQKSTHKYYCI | -0.49 | 0.699 | 3 | 3 |
| 82 | G7JR68 | CPKPPRINIRINIRCRKGFC | -0.485 | 0.974 | 3 | 6 |
| 83 | G7KV48 | CAYPHVLRCIGKNCAENKNG | -0.48 | 0.856 | 3 | 3 |
| 84 | G7LJB5 | CRPGRIPKCIFGHCNCVKQR | -0.475 | 0.994 | 4 | 6 |
| 85 | A0A072U9N1 | CPRFKNNNVRCRKGFCVNLC | -0.475 | 0.939 | 4 | 5 |
| 86 | A0A072UJR7 | IKCKVDEDCPNVFTYSYYKC | -0.475 | 0.629 | 3 | 0 |
| 87 | A7KH67 | CKSDKDCKDIIIYRYILKCR | -0.46 | 0.892 | 3 | 3 |
| 88 | I3SFJ9 | CVTKIKCVLPRKPECRNTQC | -0.455 | 0.742 | 4 | 4 |
| 89 | A7KHD6 | CYKKYPFIPWGKVRCVKGRC | -0.445 | 0.975 | 3 | 6 |
| 90 | G7JM28 | KINNVRCRKGFCIQIHKFTP | -0.44 | 0.966 | 2 | 6 |
| 91 | A0A072U6W1 | CDSAYLPLSRSCITDKDCSR | -0.44 | 0.699 | 3 | 0 |
| 92 | A0A072U8H9 | CKDKFPGNKYPIKCINGIFY | -0.43 | 0.935 | 2 | 3 |
| 93 | A7KHA9 | KIKCVLPRTPQCRNEACGCY | -0.43 | 0.871 | 4 | 3 |
| 94 | A7KHF4 | DCPKFGRVNVRCRKGNCVPI | -0.42 | 0.882 | 3 | 4 |
| 95 | G7JP15 | RYCVYPTIPLCDVKHCRCRR | -0.42 | 0.752 | 4 | 5 |
| 96 | A0A072TXJ8 | KDCPWAKNYVLKCRKGYCVF | -0.415 | 0.936 | 3 | 4 |
| 97 | A0A072U2G4 | CKTKVDCPQHKKYIAECIFG | -0.4 | 0.69 | 3 | 3 |
| 98 | A2Q2R4 | CKQVKGYIARCRKGYCMQSV | -0.39 | 0.941 | 3 | 5 |
| 99 | A7KHC9 | CTWNLCRQPWVQKCRLHMCS | -0.39 | 0.872 | 4 | 4 |
| 100 | A7KHB8 | CEKIYPGNKKPLICSTGYCY | -0.39 | 0.775 | 3 | 2 |
| 101 | G7KWZ1 | IRCRQGLKELASDGLRVTGY | -0.375 | 0.838 | 1 | 2 |
| 102 | A0A072VJQ8 | CTRRKGFSVTCRKGFCVEFK | -0.37 | 0.86 | 3 | 5 |
| 103 | G7JLZ7 | CQRYRHKLATRMICNQGFCL | -0.37 | 0.719 | 3 | 5 |
| 104 | A7KH81 | CPKVEKPITMKCINNYCKYF | -0.36 | 0.664 | 3 | 3 |
| 105 | G7IX58 | RKCLRSTCVCRKFRFTGFYY | -0.355 | 0.933 | 3 | 6 |
| 106 | A0A072TD23 | VHCQKYKCSPGLYPTCINGW | -0.35 | 0.876 | 3 | 3 |
| 107 | A7KHB2 | KQMCHLNQTPKCLKNICKCV | -0.345 | 0.657 | 4 | 5 |
| 108 | G8A361 | CKPPFNPRCHNHICICRLWG | -0.34 | 0.798 | 4 | 5 |
| 109 | G7J0M9 | CRAGSHRVQCIKHQCKCVRI | -0.335 | 0.927 | 4 | 7 |
| 110 | A0A072UQA7 | CPKSVLRVWRCINNYCRPVR | -0.335 | 0.914 | 3 | 5 |
| 111 | A0A072UL49 | CPKSEFRKWVCINNICRKMC | -0.33 | 0.866 | 4 | 4 |
| 112 | A7KHA5 | CVTKIKCVLPRKPECRNNAC | -0.33 | 0.894 | 4 | 4 |
| 113 | A0A072TWJ1 | VNNRCYLHGKPSCLNGQCAC | -0.325 | 0.897 | 4 | 3 |
| 114 | G7J0L0 | WTKIYKCIDNKCRYSVVKGL | -0.315 | 0.892 | 2 | 4 |
| 115 | A0A072UGS0 | DCPKPLRFNIRCRKGFCVRI | -0.315 | 0.87 | 3 | 5 |
| 116 | G7IZE7 | CFIDGNCPRNMCKVRWKLRL | -0.31 | 0.877 | 3 | 4 |
| 117 | I3S866 | GLLPRCLNGWCDCSRFQPWP | -0.3 | 0.503 | 3 | 1 |
| 118 | I3SB50 | LKNEIFHWKKQICNIHFAKL | -0.295 | 0.93 | 1 | 5 |
| 119 | G7IX60 | RPQCVINTCRCRPLRFSGFY | -0.29 | 0.729 | 3 | 4 |
| 120 | A0A072UI64 | IHYYCPPSKVPYCQVDRCGC | -0.28 | 0.761 | 4 | 2 |
| 121 | A0A072VBA6 | IPRCIKYKCLCGNGVGKRWS | -0.275 | 0.988 | 3 | 5 |
| 122 | A0A072VJ77 | VYRCVGNYCRAVKIRRWNLG | -0.275 | 0.986 | 2 | 5 |
| 123 | A0A072TVW8 | CRDYLCARPTVGKCIYDYCH | -0.27 | 0.736 | 4 | 2 |
| 124 | Q1RU35 | KDCRSFLCYSPKFPVCKRGI | -0.255 | 0.905 | 3 | 4 |
| 125 | A0A072UK35 | VKIHFYCPPPKVPYCRVDRC | -0.255 | 0.673 | 3 | 4 |
| 126 | A7KHE7 | ISCKDHFECRRKINILRCIY | -0.25 | 0.915 | 3 | 4 |
| 127 | A7KHF9 | CVTRIKCVLPRKPECRNYAC | -0.25 | 0.882 | 4 | 4 |
| 128 | A7KH69 | ADCPISKLNMYNWRCIKSSC | -0.25 | 0.816 | 3 | 2 |
| 129 | A0A072UFG6 | CPKYRDLLYVFKCIDKRCEL | -0.25 | 0.617 | 3 | 2 |
| 130 | G7K748 | FYVVKCVDHKCELTKKLRRL | -0.25 | 0.59 | 2 | 5 |
| 131 | A0A072TNL0 | CEFGMIRRCISYKCQCHEAY | -0.245 | 0.596 | 4 | 2 |
| 132 | A0A072VAL4 | CPNPKYGKCLDNKCICQLIW | -0.245 | 0.894 | 4 | 2 |
| 133 | A0A072UWG7 | CKHPFKPRCLTHSCVCRLWG | -0.23 | 0.852 | 4 | 6 |
| 134 | G7KEA3 | LANKTFYLKCIDKKCEWTVT | -0.225 | 0.808 | 2 | 2 |
| 135 | A0A072VH74 | ADCLGEKCLPPKRYWCRIIT | -0.22 | 0.841 | 3 | 2 |
| 136 | A0A072VB20 | CRKHMCTPYGQLVRCINSTC | -0.21 | 0.734 | 4 | 4 |
| 137 | A0A072UBS9 | VKKPLKMWCIRQTCFYGFGK | -0.195 | 0.912 | 2 | 5 |
| 138 | A0A072TJN6 | IDKEYTVCSLHSDCKAYVCQ | -0.195 | 0.703 | 3 | 0 |
| 139 | G7KIM9 | KICHPPQIRKCVSKICKCRL | -0.19 | 0.973 | 4 | 7 |
| 140 | A0A072TR97 | CVTKVKCGLPRTPKCRNYIC | -0.19 | 0.958 | 4 | 5 |
| 141 | A0A072V6Y7 | FVRCKMNRCIYSRVQPPWAC | -0.185 | 0.868 | 3 | 4 |
| 142 | A0A072VK07 | IPYCRFREKNVRFGSPLGLC | -0.18 | 0.882 | 2 | 3 |
| 143 | A0A072VHI2 | CRTPLKPKCMYRTFCKCKVV | -0.18 | 0.818 | 4 | 6 |
| 144 | A0A072TQJ0 | CRPPLKSKCMYKTNCKCIAV | -0.17 | 0.811 | 4 | 5 |
| 145 | AFK48426 | CPKLYGANFRCRKGTCVPPI | -0.165 | 0.979 | 3 | 4 |
| 146 | G7K8T1 | RVAWCVNNKCECVLTYGPKY | -0.165 | 0.854 | 3 | 2 |
| 147 | G7KAD9 | CPKSQLEMFAWKCVKNGCHL | -0.165 | 0.804 | 3 | 3 |
| 148 | A0A072TW61 | AFYGSMRCVKGFCKHLKDVK | -0.16 | 0.919 | 2 | 5 |
| 149 | A0A072TNG5 | AQCIYPACFKDHMCRQLKCS | -0.155 | 0.832 | 4 | 3 |
| 150 | A0A072TVT5 | LKKYIYSPGARRVASSCGVP | -0.15 | 0.913 | 1 | 4 |
| 151 | A0A072V981 | CWPSFKPRCSNGWCVCDKIM | -0.145 | 0.75 | 4 | 2 |
| 152 | G7I5S5 | WFKIYRCEKGICRYHKLWIV | -0.145 | 0.872 | 2 | 5 |
| 153 | A7KHC1 | CPNDCGPHEQAKCILYACYC | -0.145 | 0.521 | 5 | 0 |
| 154 | G7J0L3 | CPPSYTKIYRCIDNKCRLVL | -0.14 | 0.773 | 3 | 3 |
| 155 | G7KPK3 | CITADDCPKVERPLKMKCIG | -0.14 | 0.584 | 3 | 1 |
| 156 | A7KHF7 | CRRNIDCPSYLCVAPKVPRC | -0.14 | 0.829 | 4 | 3 |
| 157 | G7JQH7 | GRMVCGGRSRFGFRSCGGYL | -0.14 | 0.81 | 2 | 4 |
| 158 | A7KHG6 | RCVRRMCKCLPIGWRKYFVP | -0.135 | 0.942 | 3 | 6 |
| 159 | A7KH78 | ARELPEYLKCQGGMCRLLIK | -0.13 | 0.743 | 2 | 2 |
| 160 | A0A072V8P6 | GNNLCEGRWIPKCLKPYFLF | -0.125 | 0.803 | 2 | 2 |
| 161 | A7KHE6 | LLKTYVWKCVKNECYFFAKK | -0.115 | 0.919 | 2 | 4 |
| 162 | A0A072UT45 | LRANCVDRGVCKCVPVWWRK | -0.11 | 0.933 | 3 | 4 |
| 163 | G7JM81 | CNVNNICEYNLNVDLVEEIE | -0.105 | 0.532 | 2 | -5 |
| 164 | A0A072TFE5 | CVARINCVLPRKPQCRNYAC | -0.105 | 0.941 | 4 | 4 |
| 165 | A7KH64 | IRCVTDADCPNVVKPLKPKC | -0.1 | 0.521 | 3 | 2 |
| 166 | A0A072U873 | KFPGPSKYPIKCMKGICKCV | -0.09 | 0.899 | 3 | 5 |
| 167 | G7K1L8 | CQATTKCVLPRVPRCIKYKC | -0.08 | 0.945 | 4 | 5 |
| 168 | A0A072V6P6 | LKLKCDRHTILTCFWRHCYC | -0.08 | 0.782 | 4 | 5 |
| 169 | A7KH75 | VSCKDHYDCRRKVKIVGCIF | -0.075 | 0.866 | 3 | 4 |
| 170 | A7KH74 | GVFVSCNSHIHCRVNNHKIG | -0.075 | 0.796 | 2 | 5 |
| 171 | A0A072UWC9 | CKNPDDVPRCIFPVCHCIKS | -0.07 | 0.665 | 4 | 2 |
| 172 | G7IW93 | IFLCSFIAAKNIDGRNNPTR | -0.07 | 0.873 | 1 | 2 |
| 173 | A0A072TIJ0 | IGCKTSEDCPYLGKCIEDFC | -0.07 | 0.677 | 4 | -2 |
| 174 | A0A072U9R9 | CIGYWCPLSIQPRSTKPICR | -0.065 | 0.887 | 3 | 3 |
| 175 | A7KH93 | CVYPYAVQCIHRYCKCLKSR | -0.065 | 0.798 | 4 | 5 |
| 176 | A7KHD9 | FIKCVCGVYGPIRERRLYQS | -0.06 | 0.919 | 2 | 3 |
| 177 | G7IZI9 | VISIHCRTNADCPRNMCKIG | -0.06 | 0.78 | 3 | 3 |
| 178 | A0A072VHP7 | CTNKIKCVPPRIAQCFRFKC | -0.05 | 0.983 | 4 | 5 |
| 179 | A0A072UKJ5 | CEKLYPGNKKPLICNIGYCL | -0.045 | 0.865 | 3 | 2 |
| 180 | A0A072UK90 | LCTSPNEVPECRLLKCQCIK | -0.045 | 0.615 | 4 | 1 |
| 181 | G7IVN8 | GENFCISRFRVKCWRFKCFC | -0.025 | 0.922 | 4 | 4 |
| 182 | G7I4L5 | VGCRLQREKPRCVNLVCRCL | -0.02 | 0.953 | 4 | 4 |
| 183 | A0A072UJ01 | KTLCHSPGKAKYFCSLLSLK | -0.02 | 0.907 | 2 | 5 |
| 184 | G7L4Z6 | VKVSHSHCVIDAHCPRNMCG | -0.015 | 0.608 | 3 | 4 |
| 185 | A7KHD7 | LVQRYRCINGKCNLSFVSYG | -0.005 | 0.902 | 2 | 3 |
| 186 | G7IW01 | LVQGYRCIDGKCESVFLSYR | -0.005 | 0.616 | 2 | 1 |
| 187 | A0A072U2A0 | CPKVAKINIRCRRGQCVQVF | -0.005 | 0.991 | 3 | 5 |
| 188 | A0A072UT81 | LPYCDRNRFCTSRGYIVCIT | -4.44E-17 | 0.777 | 3 | 2 |
| 189 | A7KHA7 | CYKSKKPLFKIWKCVENVCV | 0.005 | 0.924 | 3 | 4 |
| 190 | A0A072TXA9 | FCARYPSSKVDYGRCVASIS | 0.005 | 0.842 | 2 | 2 |
| 191 | A0A072TZE3 | CPYSIQPRSTKPLCRLVGGI | 0.01 | 0.858 | 2 | 3 |
| 192 | A7KH85 | CRIPLRPKCMYRHICKCKVV | 0.01 | 0.856 | 4 | 7 |
| 193 | A0A072V915 | CKARADCSKLMCELPKISWC | 0.015 | 0.839 | 4 | 2 |
| 194 | G7KT88 | FIAARVCKSDKDCKDIIIYR | 0.015 | 0.912 | 2 | 2 |
| 195 | G7JM04 | CQRYRHKLATRMVCNIGFCL | 0.015 | 0.801 | 3 | 5 |
| 196 | A2Q575 | ATLAFQAAIWKWMTPIKKSQ | 0.02 | 0.907 | 0 | 3 |
| 197 | A0A072USM4 | LGMKPKCISVLRMCKCHGWQ | 0.03 | 0.886 | 3 | 5 |
| 198 | A2Q6A5 | WICSTGEFPPRALCCRNRCV | 0.03 | 0.863 | 4 | 2 |
| 199 | A0A072UX97 | CNHPKIPKCVNNAYCKCVVA | 0.04 | 0.969 | 4 | 4 |
| 200 | G7J0N1 | FCWLDSHMQCIKHQCKCVRI | 0.04 | 0.833 | 4 | 4 |
| 201 | I3SGY2 | IINILCKTDKDCPKVQGANI | 0.045 | 0.907 | 2 | 1 |
| 202 | G7KME0 | LGEAWFKRTETGEIIWVVRC | 0.045 | 0.561 | 1 | 0 |
| 203 | A0A072UI73 | CPKNMCLLTQIPKCFKNVCK | 0.05 | 0.87 | 4 | 4 |
| 204 | A7KHG0 | VNKLRVIKCIDHICQYARNL | 0.05 | 0.939 | 2 | 4 |
| 205 | A7KH77 | CSKDECPSHLVPKCIGLTCY | 0.055 | 0.767 | 4 | 1 |
| 206 | A0A072UME6 | LLKCIHGYCVCFPRNPGDSS | 0.055 | 0.729 | 3 | 2 |
| 207 | A0A072VS71 | CRKVRGVNLRCRNGHCVMIL | 0.06 | 0.925 | 3 | 6 |
| 208 | A0A072UG60 | QIVKYYCIADQCFYYIKHIK | 0.06 | 0.557 | 2 | 3 |
| 209 | A0A072TGJ1 | AKIDYVLTLKPQCRNYTCVC | 0.07 | 0.824 | 3 | 2 |
| 210 | G7IDP6 | ISCVSDDDCPKVPYPLYIKC | 0.07 | 0.709 | 3 | -1 |
| 211 | A0A072VTT4 | VCPPNNFVRCIRNLCKCRSL | 0.07 | 0.969 | 4 | 4 |
| 212 | A7KH73 | GCSFREIPQCINSICKCMKG | 0.07 | 0.924 | 4 | 2 |
| 213 | G7L160 | CPTDLTLKCINLTCQCTSEY | 0.075 | 0.527 | 4 | -1 |
| 214 | A0A072UBT3 | CPKVVKPNYTMCAGGICWQS | 0.08 | 0.876 | 3 | 2 |
| 215 | A0A072TXW5 | CIRRAGMNIRCRKGYCVNLI | 0.085 | 0.99 | 3 | 5 |
| 216 | A0A072VGB5 | CRGPMRAKCISKAICKCRLA | 0.105 | 0.99 | 4 | 6 |
| 217 | A0A072U1J3 | KKPLCYLFASSPFERKIGVC | 0.125 | 0.551 | 2 | 3 |
| 218 | A0A072UW93 | CPPIKFAKYLCINYKCRKIC | 0.125 | 0.933 | 4 | 5 |
| 219 | A0A072UKX9 | ANCPKVISPCHTKCFDGFCG | 0.13 | 0.806 | 4 | 2 |
| 220 | A0A072UQQ6 | LYILSRCVNNICEWVKKPRI | 0.13 | 0.896 | 2 | 3 |
| 221 | A0A072U7M5 | CRQYLCIPPEFPRCIGGICR | 0.135 | 0.847 | 4 | 2 |
| 222 | A0A072UFK5 | EIGVRKICIREVCRYFAKIH | 0.14 | 0.843 | 2 | 4 |
| 223 | A0A072TH33 | RFMCIPPEKPKCVDLWCKCI | 0.14 | 0.604 | 4 | 2 |
| 224 | G7JIH1 | ISGNLARASRKKPVDVIPCI | 0.14 | 0.958 | 1 | 3 |
| 225 | G7ZZE8 | IPCKYNHDCPTILDYISICP | 0.14 | 0.562 | 3 | 0 |
| 226 | A7KHG4 | CPISQLKIYAWKCVKNGCHL | 0.16 | 0.983 | 3 | 4 |
| 227 | A0A072U1J7 | VKDCPKTVFPIGYKCIKNLC | 0.165 | 0.94 | 3 | 3 |
| 228 | A0A072TE63 | CPTGSKPKCVDQVCECILIR | 0.165 | 0.76 | 4 | 1 |
| 229 | A2Q6D2 | CSHIRDVICIFKKCKCAGGR | 0.165 | 0.986 | 4 | 5 |
| 230 | A0A072TY61 | IGGMFTLKQKKAAKVARSCV | 0.17 | 0.981 | 1 | 5 |
| 231 | G7IW95 | CPSYLVVKCLRSNCKCVRPG | 0.17 | 0.955 | 4 | 4 |
| 232 | A2Q6D8 | CVYPKSMKCIDKKCICVGAR | 0.17 | 0.925 | 4 | 4 |
| 233 | G7K901 | CPKIPSLYPTIYKCLDGICR | 0.175 | 0.792 | 3 | 2 |
| 234 | A7KHD8 | FLSIAVSITGNLARASRKKP | 0.18 | 0.971 | 0 | 4 |
| 235 | A0A072TDM7 | ICLPPKKHWCNILELVRING | 0.18 | 0.923 | 2 | 3 |
| 236 | A0A072TZB3 | GFCVNSGGATQKCLGCPSLK | 0.18 | 0.872 | 3 | 2 |
| 237 | A0A072UVS0 | VECPQYSCLRGLKMKCICFK | 0.185 | 0.899 | 4 | 3 |
| 238 | A7KHB7 | CVGKITCVLPQKPECWNYAC | 0.185 | 0.834 | 4 | 1 |
| 239 | A0A072U9K0 | VSGGFGKCVRDADCVDEVCS | 0.185 | 0.663 | 3 | -2 |
| 240 | A0A072TVQ9 | NCRVKEVGMCYFTKCYCIRL | 0.195 | 0.815 | 4 | 3 |
| 241 | A0A072U6V1 | CPEKFCSSPDVVRCIYIECY | 0.195 | 0.684 | 4 | -1 |
| 242 | A0A072ULG5 | IGECYSLYKGKFSLSIISKT | 0.195 | 0.807 | 1 | 2 |
| 243 | A0A072VIU4 | FFVCPPNNFVRCIRNLCKCR | 0.2 | 0.922 | 4 | 4 |
| 244 | G7IWA5 | LVAKCIKKLCSCRKPGLQIQ | 0.2 | 0.997 | 3 | 5 |
| 245 | G7L5G4 | CIFYGNRIMYRGSRLGICKC | 0.2 | 0.936 | 3 | 4 |
| 246 | G7K8Y8 | VSNPPLYFKCIDRGCRIVIK | 0.2 | 0.918 | 2 | 3 |
| 247 | G7IVN7 | CKEAIDCGINFCIRPFKAKC | 0.2 | 0.917 | 4 | 2 |
| 248 | A0A072U8T2 | NLVCKPGYKLGCSANYQCIC | 0.2 | 0.89 | 4 | 2 |
| 249 | A7KHB6 | FLSLFLMGTSGMKNGCKHTG | 0.21 | 0.765 | 1 | 3 |
| 250 | A0A072UIM7 | ICSPHEHSKCILYVCYCVDK | 0.21 | 0.757 | 4 | 2 |
| 251 | A0A072U8J4 | IKKCSSSCRIKCIDFRCLCP | 0.22 | 0.923 | 5 | 4 |
| 252 | G7KA19 | ADCPKSQVNSFVIKCIKNLC | 0.22 | 0.931 | 3 | 2 |
| 253 | G7KZ18 | CCKALTGADLQCLCSYKNSA | 0.225 | 0.834 | 4 | 1 |
| 254 | A0A072UB58 | FCGIWLSKYACGKCLNVTNR | 0.23 | 0.961 | 3 | 3 |
| 255 | A0A072U9D0 | LRDLVVKCIEGYCKAILYRK | 0.245 | 0.902 | 2 | 3 |
| 256 | A0A072TZS7 | KILPIIHKCINNFCKLKLYN | 0.25 | 0.958 | 2 | 5 |
| 257 | A0A072UAF0 | GNTFFIHFKFRSLLCKKHFI | 0.255 | 0.823 | 1 | 6 |
| 258 | G7IGL5 | CGDKIKCVPPRIALCINYKC | 0.26 | 0.947 | 4 | 3 |
| 259 | G7JAL7 | LVSTKILEKHTNKCAATVGL | 0.26 | 0.799 | 1 | 3 |
| 260 | A0A072VID4 | IKCVLPRVAKCVRYKCDCVR | 0.265 | 0.962 | 4 | 5 |
| 261 | A0A072V8H8 | CKTKIKCVLPRIAECVRFKC | 0.27 | 0.942 | 4 | 5 |
| 262 | G7K731 | LFYVFICKNRICELINKYPQ | 0.27 | 0.777 | 2 | 2 |
| 263 | A0A072TEN9 | IIFLSPFLIGRKGGPPGGRT | 0.275 | 0.986 | 0 | 3 |
| 264 | A0A072UAP3 | CHKVKKPLLLTCIDGICQYT | 0.275 | 0.853 | 3 | 3 |
| 265 | G7IW98 | IIFISSFIVSKRGGKDKCFR | 0.285 | 0.985 | 1 | 4 |
| 266 | G7K1M3 | WSKFFIYKCVNHVCDSISKV | 0.285 | 0.771 | 2 | 3 |
| 267 | A0A072UC03 | CATWINTCIKFKCYCIRPWG | 0.29 | 0.951 | 4 | 3 |
| 268 | G7L6H2 | LLCRNKSELPKCIAGFMCRC | 0.29 | 0.908 | 4 | 3 |
| 269 | G7IX41 | CAKGFHKLCSFGHCYCITGP | 0.295 | 0.965 | 4 | 4 |
| 270 | G7LDP2 | CNVLYPMYINRRLRCIQGIC | 0.305 | 0.848 | 3 | 3 |
| 271 | G7K1L2 | VKCMLPRIPRCIKYQCLCGY | 0.31 | 0.856 | 4 | 4 |
| 272 | A7KHF0 | CVYPDVFQCINNICKCVSHH | 0.32 | 0.694 | 4 | 2 |
| 273 | G7LG17 | CDPPEYPRCLGILCKCVYVS | 0.325 | 0.661 | 4 | 0 |
| 274 | A7KHB5 | IFISSFIVSKSLNGGGKDKC | 0.325 | 0.97 | 1 | 2 |
| 275 | G7K5Z0 | CPPNYSFLFAIRCIKQKCVT | 0.33 | 0.95 | 3 | 3 |
| 276 | G7IIW2 | KSTCLPPQIPKCLRMICECV | 0.355 | 0.666 | 4 | 2 |
| 277 | A0A072VI94 | VRCIRNLCKCRFVYLNTFLK | 0.355 | 0.983 | 3 | 5 |
| 278 | G7K5D9 | CPQLNSEIFAFKCIEKLCKL | 0.36 | 0.758 | 3 | 1 |
| 279 | A7KHA1 | QRRKKSMAKMLKFFFAIILL | 0.365 | 0.702 | 0 | 6 |
| 280 | G7IZF0 | CKKVDQIPKCVGGLCKCFPI | 0.37 | 0.98 | 4 | 3 |
| 281 | A0A072VIH1 | TSFCIPPQIPKCRTICECIT | 0.385 | 0.759 | 4 | 1 |
| 282 | A0A072TR69 | CVRFRCKCVPIGWKNLSHVL | 0.39 | 0.986 | 3 | 5 |
| 283 | A0A072UDU7 | AKVLKCTPPEVVKCTCLCGK | 0.395 | 0.908 | 4 | 3 |
| 284 | A0A072UWP9 | CIFTRYFPIYLGGICGCDRK | 0.395 | 0.851 | 3 | 2 |
| 285 | G7KWW8 | IFGQIPKEIGKSLNLKFLSL | 0.405 | 0.971 | 0 | 2 |
| 286 | A7KHF3 | IGRKKKGETPKLVYVIILFL | 0.405 | 0.97 | 0 | 4 |
| 287 | G7JUV6 | IFTKVVGQKFFSFSLDGKCG | 0.41 | 0.875 | 1 | 2 |
| 288 | G7IX29 | GCCIHGRCWCFNSPIFADKI | 0.41 | 0.716 | 4 | 2 |
| 289 | A0A072U2G2 | CKTKADCLQHIYYIVECIFG | 0.42 | 0.666 | 3 | 1 |
| 290 | A0A072TY82 | CIAQGFVRGGDCIKEDACCC | 0.425 | 0.704 | 5 | -1 |
| 291 | G7KA17 | CPQLSLRFFAIKCRENVCIY | 0.43 | 0.918 | 3 | 2 |
| 292 | A0A072UJS9 | CRGICLNSRCVCMMRLGWTY | 0.43 | 0.837 | 4 | 3 |
| 293 | A0A072UIP1 | FLSIIACNSSFITFRDSRCK | 0.435 | 0.81 | 2 | 2 |
| 294 | A0A072UIM9 | FLCIIVSNSSFSKTFDRACK | 0.45 | 0.83 | 2 | 2 |
| 295 | G7KAI9 | CKWPFIVQCYKNVIKIGPVG | 0.45 | 0.987 | 2 | 3 |
| 296 | G7K1L6 | CIFDIDCPTKKCAPPLVAKC | 0.46 | 0.731 | 4 | 1 |
| 297 | A0A072VAZ7 | VCLNYKFPTCVGKKCYCLSA | 0.465 | 0.973 | 4 | 3 |
| 298 | A0A072UA67 | CYKIYGIPLDGVWRCVKGFC | 0.47 | 0.814 | 3 | 2 |
| 299 | A0A072TRV3 | CYNQLSCIIEEICLDGSCHC | 0.48 | 0.665 | 5 | -2 |
| 300 | A0A072UYP4 | CYDQITCIIGDVTCLEGSCD | 0.485 | 0.64 | 4 | -4 |
| 301 | A0A072V9I4 | LINGGSVPCLTSFGCPRSTC | 0.52 | 0.798 | 3 | 1 |
| 302 | A0A072V4N7 | CPNVCVDFLISRCINNKCYC | 0.52 | 0.791 | 5 | 1 |
| 303 | A0A072V8L4 | ANCPCVFPLKPRCNFGYCIC | 0.525 | 0.817 | 5 | 2 |
| 304 | A0A072VUP8 | VRLPLVPISVGKSIQRFARF | 0.525 | 0.989 | 0 | 4 |
| 305 | A0A072V8Q0 | FISLFFIAKNVEGRVKCIKD | 0.525 | 0.942 | 1 | 2 |
| 306 | I3SVG9 | FLSITVYGYIPGIVNKPCKT | 0.545 | 0.911 | 1 | 2 |
| 307 | I3SP41 | QQILPRYVLCVNGLCRIYFP | 0.545 | 0.791 | 2 | 2 |
| 308 | G7IW92 | FMCPSYLAVKCIGRLCRCGR | 0.545 | 0.972 | 4 | 4 |
| 309 | A7KHD0 | KGMCKFPFIVRCLMDQCKCV | 0.545 | 0.716 | 4 | 3 |
| 310 | A0A072UTM9 | INFCPPGTAPKCFHGLIKCV | 0.555 | 0.878 | 3 | 3 |
| 311 | A7KHB0 | INGVKSLLLIKVRSFIPCQR | 0.56 | 0.998 | 1 | 4 |
| 312 | A0A072VGT6 | FLSLFLVATNIEGKFQKCCK | 0.56 | 0.935 | 2 | 2 |
| 313 | A0A072U9Q9 | ICVSDKECPTASYPLVCKCI | 0.57 | 0.838 | 4 | 0 |
| 314 | A0A072V961 | CVDSFCVPPNVPKCRVVCKC | 0.57 | 0.779 | 5 | 2 |
| 315 | A0A072TQP5 | GLAPKCFVSFALARFLSEGR | 0.575 | 0.931 | 1 | 2 |
| 316 | A0A072US80 | GLRRASTITALPINVFAIYK | 0.58 | 0.937 | 0 | 3 |
| 317 | A0A072UIG4 | LIKIVHLWKKIRVRVDVVKA | 0.585 | 0.957 | 0 | 6 |
| 318 | G7JTI3 | CQSIGCLSHLKPKCTMLGFF | 0.585 | 0.851 | 3 | 3 |
| 319 | A0A072UMF5 | FGLGRSEAINLNAKNGIIVI | 0.59 | 0.9 | 0 | 1 |
| 320 | A7KH94 | CTGPQIPKCVSHICFCLSSG | 0.605 | 0.939 | 4 | 2 |
| 321 | A7KHC8 | CPPDMCTLGVIPKCSRFTIC | 0.62 | 0.669 | 4 | 1 |
| 322 | A0A072V9E8 | CSAENCMVCINFACKCKYSV | 0.625 | 0.867 | 5 | 1 |
| 323 | A0A072TC73 | IIYVSLYLVVIEGKDGCKTK | 0.63 | 0.787 | 1 | 1 |
| 324 | A0A072UMU2 | IAAKNVVKNPGPCFVMGACS | 0.63 | 0.981 | 2 | 2 |
| 325 | A0A072UE17 | CPEISIFSPFFYKCINNGCV | 0.635 | 0.73 | 3 | 0 |
| 326 | G7JP95 | VPLPRFLKCIANLCCLVRKK | 0.64 | 0.987 | 3 | 5 |
| 327 | A0A072VHX2 | IATGARRKNFFFIILKFSSL | 0.645 | 0.992 | 0 | 4 |
| 328 | G7JAL3 | VRCIKETCKCIKILEPINVV | 0.645 | 0.89 | 3 | 2 |
| 329 | A0A072ULJ0 | IIYVSLYLVVIEGKDGCKTN | 0.65 | 0.775 | 1 | 0 |
| 330 | G7J0L2 | LFLLHIEKSSGVLIDCKTVK | 0.665 | 0.702 | 1 | 2 |
| 331 | A0A072UE40 | FLFAMNVAGFGWKCIKRRCV | 0.68 | 0.983 | 2 | 4 |
| 332 | G7I793 | LISWTRNNLYIILGLSLFSR | 0.68 | 0.848 | 0 | 2 |
| 333 | A7KHF5 | LFLSLLLVVMGGIRKKECRQ | 0.685 | 0.817 | 1 | 3 |
| 334 | G7IJE8 | FNCPSSLCTFPLKLLKPICT | 0.685 | 0.827 | 3 | 2 |
| 335 | A7KH92 | IGRKKMGETPKLVYVIILFL | 0.695 | 0.833 | 0 | 3 |
| 336 | A0A072U1S4 | LPALQRAVMKCIQGFCKIHI | 0.705 | 0.973 | 2 | 4 |
| 337 | A0A072TFD7 | CPQHLCHELIIPRCKIGVCV | 0.705 | 0.697 | 4 | 3 |
| 338 | A0A072UEN9 | RSVFCMLATALNSLTWQRLL | 0.705 | 0.679 | 1 | 2 |
| 339 | A0A072TJN1 | QPWCKLVRLQLLFHGSLIGL | 0.705 | 0.873 | 1 | 3 |
| 340 | G7K947 | CRTVADCPKLISSKFVIKCI | 0.71 | 0.933 | 3 | 3 |
| 341 | G7KU03 | KCIMLKNLSIFSNSGICSCT | 0.71 | 0.856 | 3 | 2 |
| 342 | A7KHC6 | LFCVLPNVPKCIGSKCHCKL | 0.715 | 0.929 | 4 | 4 |
| 343 | A0A072TS84 | CPHNLCFPSKAVCISSQCIC | 0.72 | 0.933 | 5 | 2 |
| 344 | A0A072TR46 | AFIIFLSIPLPPTRKTIPCK | 0.73 | 0.84 | 1 | 3 |
| 345 | A0A072UE21 | FSCLYLFMVTKEVYAKSICK | 0.735 | 0.719 | 2 | 2 |
| 346 | A7KHH0 | VILLLTIFHVSAKKKRYIEC | 0.735 | 0.786 | 1 | 4 |
| 347 | A7KHE1 | CVTPGIPKCTGYVCFCFENL | 0.735 | 0.681 | 4 | 0 |
| 348 | A0A072V9B5 | GNTTFILFKFLSLFSKQYFI | 0.745 | 0.628 | 0 | 2 |
| 349 | G7JBW1 | FGLVVRCITHHCKCIKILNP | 0.745 | 0.973 | 3 | 5 |
| 350 | G7KM16 | CIFITRVVPRLRRMGLCSCS | 0.745 | 0.908 | 3 | 4 |
| 351 | G7JJW3 | IIIFLFITEIKGDKFVFDKN | 0.745 | 0.755 | 0 | 0 |
| 352 | A0A072U8P7 | LGCKVGKVPFLYLGLPVGGN | 0.765 | 0.992 | 1 | 2 |
| 353 | A0A072VI56 | CMPGIKPVCSEGWCDCIGFI | 0.77 | 0.661 | 4 | -1 |
| 354 | A7KHB1 | LNKLLIIKCINHVCQYVGNL | 0.77 | 0.961 | 2 | 3 |
| 355 | A0A072V5Q9 | LFLVVTNAGKPFLCTILKKK | 0.775 | 0.95 | 1 | 4 |
| 356 | A0A072VI88 | IIFFSLFLVVINGRSACNRN | 0.775 | 0.947 | 1 | 2 |
| 357 | A0A072UWX6 | CISLFLVAKNINAIHCNDVN | 0.775 | 0.848 | 2 | 1 |
| 358 | A0A072V7Q8 | VKTKLSMCFLGLRNVAITNV | 0.78 | 0.818 | 1 | 3 |
| 359 | G7IX62 | CVNLILCDFDEKPKCIINIC | 0.785 | 0.744 | 4 | -1 |
| 360 | G7LJA1 | LMKCVPGKVNVCSLGRCYCV | 0.8 | 0.962 | 4 | 3 |
| 361 | A0A072UU05 | ILTPNARKNFLALMHIICGA | 0.81 | 0.861 | 1 | 3 |
| 362 | A7KHC4 | ICSVHAVTKCIGNMCRCLAN | 0.81 | 0.956 | 4 | 3 |
| 363 | A7KHD2 | IIFFSQIIVATNAQKIRRCF | 0.815 | 0.93 | 1 | 3 |
| 364 | A0A072UJJ3 | CVDGFCDVTVKEITKSCFIC | 0.815 | 0.733 | 4 | -1 |
| 365 | A0A072UKL5 | AIIYVSMYLVVIEGKDGCKT | 0.82 | 0.71 | 1 | 0 |
| 366 | A7KHA6 | LVANGLKIFCIDVADCPKDL | 0.82 | 0.758 | 2 | -1 |
| 367 | G7IYS3 | LLYKCIYNKCIVFTRIPFPF | 0.82 | 0.748 | 2 | 3 |
| 368 | A0A072U6R7 | IIIYLFLLRVVAKDLHRRQL | 0.83 | 0.943 | 0 | 4 |
| 369 | A0A072TVX6 | MGETILVGVVKAIANRVH | 0.833333 | NA | 0 | 3 |
| 370 | G7JZA9 | CLPSLVSKCINFICECTHSM | 0.835 | 0.701 | 4 | 1 |
| 371 | A7KH89 | IFVSLFLVATKGGSKPFLTR | 0.84 | 0.846 | 0 | 3 |
| 372 | G7L0W3 | FLSLFLVIINCRFELKTSSK | 0.845 | 0.918 | 1 | 2 |
| 373 | G7JZB1 | LTIFISLLLIETIRRLQCKH | 0.85 | 0.857 | 1 | 3 |
| 374 | A0A072V827 | IFLYLFHVATTNRFLYRIGC | 0.86 | 0.822 | 1 | 3 |
| 375 | G7KPY9 | VINVGGKCISHMHCIYLSCG | 0.86 | 0.785 | 3 | 3 |
| 376 | A7KH70 | ILCLFPIIKRCIHNHCKCVP | 0.86 | 0.932 | 4 | 5 |
| 377 | G7L4D1 | FCSTFEILSIERKVGVCECI | 0.86 | 0.671 | 3 | -1 |
| 378 | A0A072TF59 | FLSIIISNSSFGMIFDRACK | 0.87 | 0.74 | 1 | 1 |
| 379 | A0A072UGC5 | ILYLFLLHVIAEDFPFHKCE | 0.87 | 0.524 | 1 | 0 |
| 380 | A0A072VH59 | IVLFLSLFLATKNIDGRRVS | 0.87 | 0.891 | 0 | 2 |
| 381 | A0A072U105 | FLVIRVSDSIPYVNIGPCVK | 0.875 | 0.751 | 1 | 1 |
| 382 | G7I616 | LVVVCHDHICKCLRLIKIRS | 0.875 | 0.848 | 3 | 5 |
| 383 | A0A072V580 | LYIACVAHECQCVHLQSALT | 0.875 | 0.715 | 3 | 1 |
| 384 | G7KV47 | LSVLLVVIEGYPFQECKVDA | 0.89 | 0.602 | 1 | -2 |
| 385 | A0A072UKE4 | IIVCDSSIIFLRCITDKDCP | 0.9 | 0.737 | 3 | -1 |
| 386 | A0A072U652 | IYLFLLHVVAKDLPFNICEK | 0.905 | 0.628 | 1 | 1 |
| 387 | A0A072UE15 | CKPFCNQAFLSCCAFGQCIC | 0.905 | 0.902 | 6 | 1 |
| 388 | A0A072UZ22 | GLVVRCITRQCKCITILNPI | 0.91 | 0.994 | 3 | 3 |
| 389 | A0A072UEW4 | GFVVAVKCVRRLCIYNVHLH | 0.91 | 0.94 | 2 | 5 |
| 390 | A0A072UC74 | ILLSQFLVKKAEITNIPCVS | 0.91 | 0.92 | 1 | 1 |
| 391 | A0A072TIH6 | LQPKCIVLEILPHSLSGGIC | 0.91 | 0.902 | 2 | 1 |
| 392 | A0A072V3F1 | LLFLFRLLIVKEVSGKSKLY | 0.915 | 0.938 | 0 | 3 |
| 393 | A7KH72 | LIIFLSIYVGVNDCKRIPCK | 0.92 | 0.838 | 2 | 2 |
| 394 | A0A072V0N4 | VYVTIIFLYMFHISTNIEGK | 0.92 | 0.514 | 0 | 1 |
| 395 | G7KEA5 | LKFVVTKKKTLKFVYAMILF | 0.92 | 0.91 | 0 | 5 |
| 396 | A7KHC0 | VFLFLSIFLSAGNSKSYGPC | 0.925 | 0.724 | 1 | 1 |
| 397 | A7KHE3 | IICIYLFIVITTRKTDIRCR | 0.925 | 0.793 | 2 | 3 |
| 398 | A0A072U6R1 | ICATHGISKCVATMCFCNLN | 0.93 | 0.876 | 4 | 2 |
| 399 | A0A072TZ06 | VLFLFISTPFIIKKPGSPNL | 0.935 | 0.854 | 0 | 2 |
| 400 | A0A072UZJ3 | AKCFVSFALARFLSKGKCLC | 0.935 | 0.992 | 3 | 4 |
| 401 | A0A072TQ17 | LIIFLSLFLVESKQTNIPCK | 0.94 | 0.78 | 1 | 1 |
| 402 | G7KA71 | ISFISLFFIAKNDAVYIKCK | 0.945 | 0.935 | 1 | 2 |
| 403 | A7KH90 | IQMGKNMAQRFMFIYALIIF | 0.945 | 0.669 | 0 | 2 |
| 404 | G7KRA4 | KLHFPLLFLLLTLFTTKPLQ | 0.95 | 0.67 | 0 | 3 |
| 405 | A0A072UIZ7 | CLFPYLVVTTKTAIACVTNK | 0.955 | 0.817 | 2 | 2 |
| 406 | A0A072TS18 | AFIIFLSIHLPTVRSDIPCK | 0.955 | 0.645 | 1 | 2 |
| 407 | A0A072U2A2 | FLVIIVSHSVTSPWVLKQHC | 0.965 | 0.727 | 1 | 3 |
| 408 | A0A072TYZ0 | IYKCIPPAKPKCVLFGCMCI | 0.99 | 0.882 | 4 | 3 |
| 409 | A0A072TVV1 | LIFLTLFVVALSNDTEYTDC | 0.99 | 0.513 | 1 | -3 |
| 410 | G7KV27 | ILFISLLLVVTKGYREPFSS | 0.995 | 0.739 | 0 | 1 |
| 411 | A0A072U1H1 | VILVSLFFVIANSRGIRPGR | 1.005 | 0.916 | 0 | 3 |
| 412 | G7JYH7 | GRNMTIKTLKFVYVIILFFS | 1.005 | 0.867 | 0 | 3 |
| 413 | G7JBD8 | FSLFLVAKGDDVKIKCVSAI | 1.01 | 0.927 | 1 | 1 |
| 414 | A0A072UUT3 | IFLSLFLVTTKAAERIYRCL | 1.01 | 0.859 | 1 | 2 |
| 415 | A0A072UBM1 | VCSVPGCSNICTLPDVPTCI | 1.01 | 0.742 | 4 | -1 |
| 416 | A0A072V8K9 | IIFLSISFSITNSFKMFCRY | 1.015 | 0.673 | 1 | 2 |
| 417 | G7IX56 | IIILSLFQLSINAREKVNCL | 1.025 | 0.816 | 1 | 1 |
| 418 | A7KHE0 | IICVFLLNIAAQEIENGIHP | 1.025 | 0.663 | 1 | -1 |
| 419 | G7INW8 | FIFLIILSAKVRGAHIKCET | 1.03 | 0.943 | 1 | 3 |
| 420 | A7KHC7 | FILISLFLVVTNANANNCTD | 1.04 | 0.664 | 1 | -1 |
| 421 | G7JLV0 | ILFLFLFFVTTEACGGKTHY | 1.04 | 0.638 | 1 | 1 |
| 422 | G7K966 | CPYLISRTLVIMCINKQCVA | 1.045 | 0.913 | 3 | 2 |
| 423 | A0A072TT06 | FVYAFFIFLSIAHRPPANTI | 1.045 | 0.742 | 0 | 2 |
| 424 | A0A072ULV0 | IGLIPCVSDADCPEELALVM | 1.045 | 0.971 | 2 | -4 |
| 425 | A7KH95 | LRRKNTVQILMFVSALLIYI | 1.045 | 0.724 | 0 | 3 |
| 426 | A7KHD4 | IFMLPFVMRCINFRCQIVNS | 1.05 | 0.759 | 2 | 2 |
| 427 | A0A072TPM2 | FVYAFIIFLSIHFPPRIKCN | 1.055 | 0.764 | 1 | 3 |
| 428 | A0A072TI02 | IIFLSLSLVAIEAGRGYRCT | 1.055 | 0.752 | 1 | 1 |
| 429 | A0A072UBI5 | VLSDGVCMSLSGTFNGLCIP | 1.055 | 0.723 | 2 | -1 |
| 430 | G7IQH9 | GGNMTNIIKFVKVMIYFLSI | 1.06 | 0.815 | 0 | 2 |
| 431 | A7KHG7 | ILFVSLCLVVVDGESKLEQT | 1.06 | 0.661 | 1 | -2 |
| 432 | A7KHG1 | FIFLIILPAKIRGETLSLTH | 1.065 | 0.778 | 0 | 2 |
| 433 | G7KNS7 | IFLSLFLVESEKLDIRCATV | 1.065 | 0.692 | 1 | -1 |
| 434 | A7KHG3 | LLNGKIIYLLCLKKKKFLII | 1.07 | 0.979 | 1 | 5 |
| 435 | A0A072U0S0 | CPEAVFFVTFRCIKNICVRI | 1.08 | 0.796 | 3 | 2 |
| 436 | G7K4P1 | FIFLIILPAKIRGEVFQRVT | 1.08 | 0.883 | 0 | 2 |
| 437 | A0A072UVS8 | LRRKNTVQILMFVSALIIYI | 1.08 | 0.731 | 0 | 3 |
| 438 | A7KH63 | FFYALIIFLSPFLVDRRSFP | 1.09 | 0.623 | 0 | 1 |
| 439 | A0A072TJN4 | IFLTLFVVALSDDSKPFSSL | 1.095 | 0.612 | 0 | -1 |
| 440 | G7KEA6 | IQFISLFLITIEVGRLRYGC | 1.105 | 0.645 | 1 | 1 |
| 441 | G7JLZ2 | LIILFSPFLAARLVFVNREK | 1.115 | 0.733 | 0 | 2 |
| 442 | G7IV77 | GFQIGCVRKICTCLRILAPI | 1.12 | 0.99 | 3 | 3 |
| 443 | A0A072UTT4 | IFLFLVANNVEGYILCKTVN | 1.13 | 0.676 | 1 | 0 |
| 444 | G7KYC2 | CPKAVSFLVFKCIDNICVRV | 1.14 | 0.887 | 3 | 2 |
| 445 | G7JZU8 | ILISLFLVVTNANAHNCTDI | 1.14 | 0.66 | 1 | 0 |
| 446 | A0A072UM18 | IIFLSLFLLSTNIDAAECYQ | 1.14 | 0.592 | 1 | -2 |
| 447 | A0A072U075 | ILFSLFVFFTSGAIPCGTRD | 1.145 | 0.726 | 1 | 0 |
| 448 | G7KDX9 | ILLIFVIFASDMCKKSAARG | 1.145 | 0.883 | 1 | 2 |
| 449 | A0A072VK25 | IKFILFLLDIPNIIPCKTRV | 1.15 | 0.721 | 1 | 2 |
| 450 | G7KEA2 | KILKFVYEMILFLSLFHLAR | 1.16 | 0.509 | 0 | 3 |
| 451 | A0A072UYA8 | IIIFISLFLGANVEGRIKCK | 1.165 | 0.816 | 1 | 2 |
| 452 | Q2HW73 | LSLFLVAKGDDVKIKCVVAA | 1.175 | 0.938 | 1 | 1 |
| 453 | A7KHE5 | FVHVLIIFLSLFHVVKNDDG | 1.18 | 0.748 | 0 | 1 |
| 454 | A0A072U556 | CDNVICVAGGIPKCITPFCF | 1.185 | 0.882 | 4 | 0 |
| 455 | A7KHC5 | FVSLYLVVVDGVSKLAQSCS | 1.185 | 0.809 | 1 | 0 |
| 456 | Q2HUY6 | GESMAKIVKFVYFVIIFASP | 1.19 | 0.72 | 0 | 1 |
| 457 | A0A072UUY4 | ILFLSLFLVAKNVTAQIRCN | 1.195 | 0.985 | 1 | 2 |
| 458 | A0A072UG77 | VSKFFIFSKFSCLLIHNLLY | 1.195 | 0.746 | 1 | 3 |
| 459 | G7KS99 | ILFISLLLVVTGAVRKPECR | 1.2 | 0.836 | 1 | 2 |
| 460 | A0A072UKJ7 | ASTFRLTFFFLIVNAVNIRC | 1.205 | 0.873 | 1 | 2 |
| 461 | G7J1T4 | IIFASLYYVVALVQNECVTD | 1.21 | 0.559 | 1 | -2 |
| 462 | G7JXA2 | LFITLFLVAKNVDALKKCIT | 1.21 | 0.925 | 1 | 2 |
| 463 | A0A072TFZ5 | ILVSLIIVATSHSFLPCQTK | 1.22 | 0.881 | 1 | 2 |
| 464 | A0A072TT10 | FVYAFIIFLSIPLPPARSDF | 1.22 | 0.64 | 0 | 0 |
| 465 | A0A072UTZ4 | LCLLLVTMNVNAVIKCFQDS | 1.22 | 0.702 | 2 | 0 |
| 466 | G7INE8 | CLPPLQVICGGDFLCFCIYQ | 1.22 | 0.683 | 4 | -1 |
| 467 | A0A072UJQ2 | GIRKNMAEILKFVYIMIIFL | 1.225 | 0.711 | 0 | 2 |
| 468 | G7KA66 | CMIIFLSLFLIATKVGGEHN | 1.225 | 0.704 | 1 | 1 |
| 469 | I3S2V0 | IILLSQFIVEKAEITNIPCV | 1.23 | 0.773 | 1 | -1 |
| 470 | A7KH86 | FIVLVTLFLGPKNVYAFQPC | 1.235 | 0.767 | 1 | 1 |
| 471 | A0A072VST7 | IIILNHMNVKLMKIVHKILL | 1.24 | 0.766 | 0 | 5 |
| 472 | G7K8E1 | ILLHSLFLIVIDTTNGLKCG | 1.245 | 0.884 | 1 | 1 |
| 473 | A0A072U9J8 | FSLFLVATNAGGCNPCLVTC | 1.245 | 0.859 | 3 | 0 |
| 474 | G7KU61 | AIFFVSLIFGVVSGKKKCAS | 1.25 | 0.961 | 1 | 3 |
| 475 | A7KH99 | INFLSLFLVETAITNIRCVS | 1.26 | 0.743 | 1 | 0 |
| 476 | G7JM03 | LIILFSPFLAARLVFVNPEK | 1.26 | 0.678 | 0 | 1 |
| 477 | A0A072TXJ7 | FGLLCIAFVLASGPTPSSLH | 1.265 | 0.874 | 1 | 1 |
| 478 | A0A072TTX6 | FIYSLIIFLSPFLGEAVFKR | 1.265 | 0.63 | 0 | 1 |
| 479 | A0A072VKW9 | ILVLFLFFVATKVDGAVHKE | 1.28 | 0.75 | 0 | 1 |
| 480 | A0A072UHJ2 | LIFSIFLCTSISIFACKTDK | 1.285 | 0.898 | 2 | 1 |
| 481 | A0A072THJ9 | IIFLSLFMVEANIPGARCAT | 1.285 | 0.677 | 1 | 0 |
| 482 | A0A072V668 | VSVFLIVVYGEKECISDAVC | 1.285 | 0.659 | 2 | -2 |
| 483 | G7JDN2 | VVTLIICLKLTYISPNLKYL | 1.285 | 0.84 | 1 | 2 |
| 484 | A0A072V8P5 | IIFVYSLNSVTIHMFIKLST | 1.29 | 0.507 | 0 | 2 |
| 485 | G7K1I6 | ILLVTLFLVPKNVDAFVKCE | 1.29 | 0.768 | 1 | 0 |
| 486 | A0A072UY60 | ILISLFLFSTNVDGKPIFIS | 1.3 | 0.714 | 0 | 0 |
| 487 | A0A072V9A9 | KFVKVMIYFLSIFLISTYFK | 1.3 | 0.674 | 0 | 3 |
| 488 | G7IDP5 | IIFLSLSFVVTSYRTRIPCV | 1.305 | 0.708 | 1 | 2 |
| 489 | G7KEA4 | ILFLSLFLITTNVGGSYYGC | 1.31 | 0.585 | 1 | 0 |
| 490 | G7JBC9 | ISLFLVAGGEEIIIIKCQTA | 1.325 | 0.872 | 1 | -1 |
| 491 | A0A072UBT5 | ILLSIFLVEKAEITNIPCVS | 1.33 | 0.803 | 1 | -1 |
| 492 | A0A072V830 | FLYFSNFLLYFVVHTQSFVL | 1.335 | 0.599 | 0 | 1 |
| 493 | A0A072UL33 | FFISLFLALVHVDGARFGIK | 1.345 | 0.803 | 0 | 2 |
| 494 | A0A072V9R1 | ALISFLSLILVLSSNEIEHC | 1.345 | 0.683 | 1 | -1 |
| 495 | A0A072UAN2 | VSVLIIFLSLFLADTKQTNI | 1.355 | 0.722 | 0 | 0 |
| 496 | G718L8 | LKALLIFFSLFLVEINGEVK | 1.355 | 0.704 | 0 | 0 |
| 497 | G7J0V6 | IIFLFLILDVATYAEKIRTC | 1.36 | 0.608 | 1 | 0 |
| 498 | A0A072UKS5 | ILFSPFLAALVIIDHHKPCV | 1.36 | 0.725 | 1 | 2 |
| 499 | G7KS41 | LIIFFFLFLVETKRTNIPCF | 1.36 | 0.56 | 1 | 1 |
| 500 | A0A072UB12 | IVLLSQFLVVINGSIPCETT | 1.365 | 0.811 | 1 | -1 |
| 501 | A0A072V7Y8 | IVILLLLLVATEAGTGNIRQ | 1.365 | 0.794 | 0 | 0 |
| 502 | A0A072UY86 | GKYMAQILKFVYVIIIFLSS | 1.37 | 0.667 | 0 | 2 |
| 503 | A0A072V7Y1 | ILFLFLFAINVTAFRDPCNF | 1.37 | 0.616 | 1 | 0 |
| 504 | A0A072UL23 | FFTIFSIFVFYTTFYHLTLT | 1.375 | 0.568 | 0 | 1 |
| 505 | A7KH98 | FIFLFLVAKNVKGYVVCRTV | 1.38 | 0.929 | 1 | 3 |
| 506 | A0A072TPU2 | LLFSIFLCISISIFSCKTDK | 1.38 | 0.869 | 2 | 1 |
| 507 | G7K944 | FLSLFLIAIDIKVEAFLRCD | 1.385 | 0.703 | 1 | -1 |
| 508 | A7KHF6 | ILFLSLLLVVMGGIRRFECR | 1.39 | 0.765 | 1 | 2 |
| 509 | A0A072VHS4 | IFLSLFIVAMNANAFSICQN | 1.395 | 0.649 | 1 | 0 |
| 510 | A0A072UJY0 | FPNLFYRIFYHLLVTFFIFF | 1.405 | 0.634 | 0 | 2 |
| 511 | G7KU13 | IIFLSLFLVATNVNAINKCS | 1.415 | 0.931 | 1 | 1 |
| 512 | G7L3W7 | IFISLILSVANAGKSLESDI | 1.415 | 0.923 | 0 | 0 |
| 513 | A7KH80 | LIIFFSLFPVITNGDRIPCV | 1.415 | 0.641 | 1 | 0 |
| 514 | A0A072UEA5 | ILFASLFLVAMEIGGQSFLR | 1.42 | 0.647 | 0 | 0 |
| 515 | A7KHE2 | ALIIFVSPFLLATFRTRLPC | 1.425 | 0.85 | 1 | 2 |
| 516 | A0A072V9P7 | IIIFSVFFVTTKSDSILCTT | 1.425 | 0.628 | 1 | 0 |
| 517 | A7KHA0 | FVYTLIIFLFPSHVITNKIA | 1.425 | 0.679 | 0 | 2 |
| 518 | G7J0H4 | VIVLFLSLFLAAKNIDGRVS | 1.43 | 0.84 | 0 | 1 |
| 519 | A7KHC3 | FKFVYTIIIYLFLLRVVAKD | 1.43 | 0.825 | 0 | 2 |
| 520 | A0A072U9U0 | LLSIFLIKIVSGSNTLLAFR | 1.435 | 0.944 | 0 | 2 |
| 521 | A0A072TR88 | FIYSLIIFLSLFFGEAALER | 1.435 | 0.572 | 0 | -1 |
| 522 | G7JRC1 | FLYALITFLFLFLVETSTTN | 1.44 | 0.56 | 0 | -1 |
| 523 | A7KHA3 | QIRKIMSGVLKFVYAIILFL | 1.44 | 0.838 | 0 | 3 |
| 524 | G7K8Y9 | VLFLSLFIFSIAAQNLMKCN | 1.44 | 0.715 | 1 | 1 |
| 525 | G7IX57 | LCVFGSKAECVVNICICVPP | 1.445 | 0.886 | 4 | 0 |
| 526 | G7L3W6 | IIFISLILDVTNAGPIFCYN | 1.445 | 0.65 | 1 | -1 |
| 527 | A0A072UK30 | IILFSPLIAQRIVGMSLFTP | 1.45 | 0.627 | 0 | 1 |
| 528 | A0A072VX14 | IIFISLFLVVTTHIPCVHHD | 1.47 | 0.685 | 1 | 2 |
| 529 | A7KH83 | FISLFLVSKNVAIDIFVCQT | 1.485 | 0.778 | 1 | 0 |
| 530 | I3S7Z9 | IIFLSQFLVVTSTTTFPCVS | 1.49 | 0.713 | 1 | 0 |
| 531 | A7KHB4 | IYALIIFSSLFVRDGIPCLS | 1.49 | 0.625 | 1 | 0 |
| 532 | G7IT95 | FISLILIVTSNVHSLLPCGT | 1.495 | 0.829 | 1 | 1 |
| 533 | A0A072V8L8 | VFISLFFVVRDVKAGLAHFC | 1.495 | 0.793 | 1 | 2 |
| 534 | A0A072UVD6 | FIFLFLVANNVEGYIVCITD | 1.5 | 0.556 | 1 | -2 |
| 535 | A0A072UYL2 | FIFLFLVSTNVHAGIRCVFP | 1.5 | 0.86 | 1 | 2 |
| 536 | A0A072UMS5 | VILFLSLFLVVTNVESADCD | 1.505 | 0.639 | 1 | -3 |
| 537 | G7JQH9 | IIFFSLFFVLTNGELEIRCV | 1.505 | 0.606 | 1 | -1 |
| 538 | G7K939 | VLFLSLFLIATDVKAFLKCD | 1.505 | 0.802 | 1 | 0 |
| 539 | G7IQF6 | ILFSPSLVVPLKVIIPSSTC | 1.51 | 0.833 | 1 | 1 |
| 540 | G7K8Z9 | KTIKFVYTMILFLSLFIVAK | 1.51 | 0.703 | 0 | 3 |
| 541 | G7KJJ7 | KIFKFIYGLVIFLYLFLIQK | 1.52 | 0.568 | 0 | 3 |
| 542 | A0A072U8A7 | FDPILVTSFYYLLFFLTAVT | 1.525 | 0.636 | 0 | -1 |
| 543 | A0A072UEE8 | ILFIFLFLVAYKIEALTKCE | 1.53 | 0.586 | 1 | 0 |
| 544 | A0A072UA23 | ILINISLFFVEATELNIPCV | 1.53 | 0.597 | 1 | -2 |
| 545 | G7K5W8 | ILFLFLFLVAAEDIGGNCEC | 1.53 | 0.544 | 2 | -3 |
| 546 | G7ZXD6 | FYGSIIFLSLFLLAAFFEKG | 1.535 | 0.578 | 0 | 0 |
| 547 | G7JDM1 | ILFVSLLLIVVASERECVTD | 1.54 | 0.714 | 1 | -2 |
| 548 | G7JQH0 | ILFISLILVVTGIKADTSCH | 1.545 | 0.902 | 1 | 1 |
| 549 | A0A072VA98 | IFYAFIIFLCVFFVPTKSSK | 1.55 | 0.639 | 1 | 2 |
| 550 | G7KNA1 | IFLSLFLIEASIKTKIACVT | 1.55 | 0.893 | 1 | 1 |
| 551 | G7K955 | LFLFLIAMNVNALYVCRKVA | 1.55 | 0.646 | 1 | 2 |
| 552 | G7ISZ8 | CAIILFLSLFLVTYFERFGP | 1.555 | 0.688 | 1 | 0 |
| 553 | G7K5W7 | CLTILFLSLFLVAAEEDIGG | 1.555 | 0.703 | 1 | -3 |
| 554 | G7IV70 | IVFYTLFLVATEIVSGIPCN | 1.555 | 0.665 | 1 | -1 |
| 555 | A7KH88 | VIILILSLFLVAKGGGKKIY | 1.56 | 0.966 | 0 | 3 |
| 556 | A0A072TS14 | FLKFVYVFIIFLAIRLPPAK | 1.565 | 0.818 | 0 | 3 |
| 557 | A0A072UW98 | IILISLFLFSTNVDGKPIFI | 1.565 | 0.699 | 0 | 0 |
| 558 | A0A072TZN8 | ILFVSLFLIVVDVCGKCNSD | 1.57 | 0.724 | 2 | -1 |
| 559 | G7L378 | VSLCLVVVDGISIYVRCAST | 1.58 | 0.736 | 2 | 0 |
| 560 | A0A072VK98 | VFLCLSIFLVVESLNFGPCN | 1.58 | 0.708 | 2 | -1 |
| 561 | A0A072TX06 | FSLVLLILFITQLHNKVAAA | 1.59 | 0.821 | 0 | 2 |
| 562 | A0A072VEF3 | ALTIFLSQLLVAASSLCISD | 1.6 | 0.831 | 1 | -1 |
| 563 | A7KHD1 | IICLFPYLVVTFKTAITCDC | 1.6 | 0.703 | 3 | 0 |
| 564 | G7JQH4 | IISISLFLVVTNGVKIPCVK | 1.605 | 0.938 | 1 | 2 |
| 565 | A0A072V920 | AFIIFLSLFFVLTKSSIPCK | 1.605 | 0.86 | 1 | 2 |
| 566 | A0A072UKH2 | ILFLSLFLFIKNVDGAFVKC | 1.605 | 0.764 | 1 | 1 |
| 567 | A0A072UW23 | FLSIFIIVTNGGLIPCVSDA | 1.61 | 0.844 | 1 | -1 |
| 568 | G7IY11 | ALTIFLSLFIVGAVRIPRPL | 1.61 | 0.945 | 0 | 2 |
| 569 | G7L162 | FYALLIFVSLFLVTTNGSLP | 1.615 | 0.705 | 0 | 0 |
| 570 | A0A072VKY3 | AKTLKVMYTMVLFFSLFLVA | 1.615 | 0.599 | 0 | 2 |
| 571 | G7KA04 | GEIIKFVYSRIIFLSLFLLA | 1.62 | 0.629 | 0 | 1 |
| 572 | A0A072VJU2 | IFLSLLLILTDGGLINGGSV | 1.62 | 0.875 | 0 | -1 |
| 573 | A0A072V6Q2 | MTSIVIKYFWALLLYL | 1.625 | NA | 0 | 5 |
| 574 | G7IT79 | ILSLFIAVTNALIFCFEDIN | 1.63 | 0.651 | 1 | -2 |
| 575 | G7JQH8 | FFSLFPVITNGGIYISFLFF | 1.63 | 0.67 | 0 | 0 |
| 576 | G7IV71 | IIVFYTLFLVGTEIVSGHAC | 1.63 | 0.658 | 1 | 0 |
| 577 | A0A072UKQ0 | VHALILFLSLFLVAKVRTPC | 1.655 | 0.895 | 1 | 3 |
| 578 | G7KI57 | FVYTLILYLFLLYVVPFHRC | 1.655 | 0.629 | 1 | 2 |
| 579 | A0A072VIP2 | VIIIFLSLFLVATNIKGKPF | 1.66 | 0.86 | 0 | 2 |
| 580 | A0A072UIP6 | IHALVIFLSLIGLVISGNHT | 1.66 | 0.754 | 0 | 2 |
| 581 | A0A072UW27 | ISVFALILILSPFLVVTDRD | 1.665 | 0.72 | 0 | -1 |
| 582 | A0A072UIG7 | VVLFFCILSFSAKTLARNIV | 1.665 | 0.958 | 1 | 2 |
| 583 | G7KJJ8 | AKTLKFLCGLVLFVYLFFIK | 1.665 | 0.824 | 1 | 3 |
| 584 | G7I794 | IVFFFIFLSVTNSSAFSGCM | 1.68 | 0.581 | 1 | 0 |
| 585 | G7JHR4 | ILVSLILVVTSHSFLPCVTK | 1.69 | 0.86 | 1 | 2 |
| 586 | A0A072UU77 | IIFFSLILAVTNAGLFRCKV | 1.7 | 0.888 | 1 | 2 |
| 587 | A7KHC2 | FIIFLSLILAVISQHPFTPC | 1.705 | 0.715 | 1 | 1 |
| 588 | A0A072VAB0 | ALIIFLSFILAVISEDIENC | 1.71 | 0.62 | 1 | -3 |
| 589 | A0A072UKF8 | ILILCVSLLLIGEASGKECV | 1.72 | 0.854 | 2 | -1 |
| 590 | G7KSG9 | FLSLFLVITNSVRIPCVTVA | 1.725 | 0.72 | 1 | 1 |
| 591 | A0A072TU94 | VQTPFFIYAFIIFLSLFPYV | 1.725 | 0.65 | 0 | 0 |
| 592 | A7KHB9 | IMILCVSLLLIAEASGKECV | 1.735 | 0.758 | 2 | -1 |
| 593 | A0A072UXR0 | FILTMILLLSLFLVAESGGK | 1.74 | 0.726 | 0 | 0 |
| 594 | A7KHA2 | LILFISLILVVTGINAEADT | 1.745 | 0.756 | 0 | -2 |
| 595 | A0A072U1G7 | LFVSIFIIVVNVGGKCVSDA | 1.745 | 0.857 | 1 | 0 |
| 596 | A0A072V566 | IILLSLFLAAIDADVVNCTS | 1.75 | 0.773 | 1 | -2 |
| 597 | A7KH65 | ILFLSLFLVQFLTCKGLTVP | 1.75 | 0.846 | 1 | 1 |
| 598 | A0A072UB79 | ALIIVLSLFLVETNTATCIT | 1.77 | 0.736 | 1 | -1 |
| 599 | A0A072UGB9 | IICLFLLQVAAQEVLVIHEC | 1.775 | 0.593 | 2 | -1 |
| 600 | A0A072VLX6 | FIVVSLFLVVTCETRIPCVS | 1.78 | 0.694 | 2 | 0 |
| 601 | G7JM79 | ALIIFISLVITGRSTINVMC | 1.805 | 0.655 | 1 | 1 |
| 602 | A0A072VDG0 | VKTLKFVNVIIFFLSLFLSA | 1.81 | 0.782 | 0 | 2 |
| 603 | A0A072V6V6 | MDALITLIVQLIYVSLHLF | 1.810526 | NA | 0 | 1 |
| 604 | A0A072TQ08 | IFLSLFLVATKAEFTLFILS | 1.83 | 0.758 | 0 | 0 |
| 605 | A0A072UES3 | CVRILFISLFLIATKFGVAS | 1.835 | 0.964 | 1 | 2 |
| 606 | A7KHG9 | FLSIFIITLQVNVVVCEIDA | 1.84 | 0.606 | 1 | -2 |
| 607 | G7IWG0 | LIIFLSIYLVVTDGIILCKD | 1.865 | 0.607 | 1 | -1 |
| 608 | A0A072TZK8 | NNIVKFGYVMIIFLSLFFVV | 1.88 | NA | 0 | 0 |
| 609 | G7IQM1 | IIILSLALVVTCNGIPICQT | 1.895 | 0.778 | 2 | 0 |
| 610 | G7LE02 | LVKFVYVMITLLSIVVVAKN | 1.9 | 0.798 | 0 | 2 |
| 611 | A7KHD5 | KIIKFVYVLAIFFSLFLVAK | 1.925 | 0.712 | 0 | 3 |
| 612 | A7KH97 | GQILIFVFALINFLSPILVE | 1.925 | 0.58 | 0 | -1 |
| 613 | G7IMB5 | GQIQKFISSLIIIISLVLVV | 2.005 | 0.701 | 0 | 1 |
| 614 | A0A072UK19 | VKVFKFTYLMIIFFSLFLVA | 2.005 | 0.603 | 0 | 2 |
| 615 | A0A072UST9 | LILFYSIFLGIIVCNSSLIS | 2.08 | 0.556 | 1 | 0 |
| 616 | G7KA73 | KILKCVYAMILFLPLFVVAM | 2.08 | 0.51 | 1 | 2 |
| 617 | A0A0C3VSU6 | IICLFLFLLHVAAQKDLIIL | 2.13 | 0.661 | 1 | 1 |
| 618 | A0A072TFS3 | FLLFIYSLIIFLSLFFGEAA | 2.165 | 0.529 | 0 | -1 |
| 619 | A0A072V8R5 | FKILLFTSSIIVFLSLFFVT | 2.17 | 0.623 | 0 | 1 |
| 620 | G7K672 | FFAIILLLSLFLVATEVGGA | 2.22 | 0.691 | 0 | -1 |
| 621 | A0A072TEH7 | AKVLKLVNVMIIFLALVLVA | 2.39 | 0.666 | 0 | 2 |
| 622 | P69136.1 | KWCFRVCYRGICYRRCR | NA | NA | 3 | 5 |
| 623 | P14215.1 | RRWCFRVCYRGFCYRKCR | NA | NA | 4 | 6 |

We claim:

1. An antibacterial pesticide composition comprising:
a peptide having an amino acid sequence selected from SEQ ID NO: 8, SEQ ID NO: 9. SEQ ID NO: 10, SEQ ID NO: 11, SEQ ID NO: 14 or mixtures thereof.

2. The antibacterial pesticide composition of claim 1 comprising:
a peptide having an amino acid sequence of SEQ ID NO: 8.

3. The antibacterial pesticide composition of claim 1 comprising:
a peptide having an amino acid sequence of SEQ ID NO: 9.

4. The antibacterial pesticide composition of claim 1 comprising:
a peptide having an amino acid sequence of SEQ ID NO: 10.

5. The antibacterial pesticide composition of claim 1 comprising:
a peptide having an amino acid sequence of SEQ ID NO: 11.

6. The antibacterial pesticide composition of claim 1 comprising:
a peptide having an amino acid sequence of SEQ ID NO: 14.

7. The composition of claim 1 wherein the peptide has an amidated CONH₂ C-terminus residue.

8. The composition of claim 1 where the peptide or peptides are recombinantly expressed in a host cell, with a plant cell being one example host cell.

9. The composition of claim 1 with a peptide having one or more amino acid residue substitutions to provide a sequence at least 85% identical to a sequence of claim 1.

10. The composition of claim 1 with a peptide having an amino acid residue substitution in one of residues 1-20 of the sequence.

11. The composition of claim 1 with a peptide having an amino acid residue substitution in two of residues 1-20 of the sequence.

12. The composition of claim 1 with a peptide having an amino acid residue substitution in three of residues 1-20 of the sequence.

13. A method for treating citrus greening disease comprising:
treatment of a plant in need thereof with a composition comprising an effective amount of a peptide with SEQ ID NO: 8, SEQ ID NO: 9, SEQ ID NO: 10, SEQ ID NO: 11, SEQ ID NO: 14 or mixtures thereof.

14. The method of claim of claim 13 with the peptide having an amidated CONH₂ C-terminus residue.

15. A prophylactic method of treatment of citrus greening disease by treatment of a plant in need thereof with a composition comprising an effective amount of a peptide with SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12. SEQ ID NO: 13, or SEQ ID NO: 14.

16. A method for killing a psyllid vector of citrus greening disease comprising:
treatment of a plant in need thereof with a composition comprising an effective amount of a peptide with SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12. SEQ ID NO: 13 or SEQ ID NO: 14.

17. The method of claim 16 where the psyllid is Diaphorina citri.

18. A method for treating citrus greening disease comprising:
treating a plant in need thereof with a symbiont forming inoculum comprising at least one polynucleotide encoding one or more phytohormone biosynthetic enzymes and a polynucleotide expressing a peptide effective in treating citrus greening disease, wherein the phytohormone biosynthetic enzyme is at least one cytokinin biosynthetic enzyme and/or an auxin biosynthetic enzyme.

19. The method of claim 18 wherein the peptide effective in treating citrus greening disease has a sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12. SEQ ID NO: 13 or SEQ ID NO: 14.

20. The method of claim 18 wherein the polynucleotides encoding a phytohormone biosynthetic enzyme and the polynucleotide of interest are comprised in a plant cell or a bacterial cell.

21. The method of claim 18 wherein the phytohormone biosynthetic enzyme is an indole-3-acetamide hydrolase (iaaH), amidase 1 (EC 3.5.1.4), a tryptophan 2-monooxygenase (IaaM) (EC 1.13.12.3), an indole-3-lactate synthase (EC 1.1.1.110), a L-tryptophan-pyruvate aminotransferase 1 (EC 2.6.1.99), a tryptophan aminotransferase-related protein 1 (EC 2.6.1.27), indole-3-acetaldehyde oxidase (EC 1.2.3.7), a tryptophan decarboxylase 1/tryptophan decarboxylase 2 (EC4.1.1.105), an isopentenyl transferase (Ipt) and/or a Tzs (EC 2.5.1.27).

22. The method of claim 18 wherein the polynucleotides encoding a phytohormone biosynthetic enzyme and the polynucleotide expressing a peptide effective in treating citrus greening disease are comprised in a single nucleic acid construct or in two or more nucleic acid constructs.

23. The method of claim 18 wherein the one or more polynucleotides are comprised in one or more vectors selected from a group consisting of a plasmid, a T-DNA, a bacterial artificial chromosome, viral vector, or a binary-bacterial artificial chromosome.

24. A symbiont forming inoculum to treat citrus greening disease comprising a polynucleotide for expression of a peptide of sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12. SEQ ID NO: 13 or SEQ ID NO: 14, and a polynucleotide encoding one or more phytohormone biosynthetic enzymes; wherein the phytohormone biosynthetic enzyme is at least one cytokinin biosynthetic enzyme and/or an auxin biosynthetic enzyme.

25. A transgenic citrus plant transformed with a recombinant construct comprising a nucleic acid that encodes a polypeptide having at least 90% identity to the sequence SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12. SEQ ID NO: 13 or SEQ ID NO: 14.

26. The transgenic citrus plant of claim 25 wherein, expression of the polypeptide confers an altered trait in the plant of resistance to citrus greening disease.

27. The transgenic citrus plant of claim 25 wherein, expression of the polypeptide confers an altered trait in the plant of treatment of citrus greening disease.

28. The transgenic citrus plant of claim 25 wherein, expression of the polypeptide confers an altered trait in the plant of decreased CLas transmission.
